SEQUENCE LISTING (60 sequences)

SEQ ID NO: 1 - DNA, 563 bp, Homo sapiens
tttttttttttggtatgggagatgttgttgacttgtggaccataaaatctatttgttgtgatttctggattttttatatcaactgtagccatgggtagattaggaccatttgggggatctcgtcttttctcagatttcctttgtcgacggcccaaaaggtctgtaactgcttttcgaaatttttttgcttcttcttcattggcaaaattaagagcaacttgacaagtatctccagcaaaggtatgaaaatatcctctaggactattatatacaaagttattgtatagctcttgttcccacaatagtttcccatccttaatgtcaaatattcttaaaaaataagatctctgtggattgtccttaacaagacaagcaacaccactgcacttctttgaccacatacagttccgatctgctgcatataactgcaccactgctgaagacatagtcacacatttcttgccgaggaaagtgaagagggactcgttctcctgcggggtgagcaacagggaccccacgttggtgaccctccgcggcggcggcggctgctgctggacggagct

SEQ ID NO: 2 - protein, 505 aa, Homo sapiens
MSSVQQQPPPPRRVTNVGSLLLTPQENESLFTFLGKKCVTMSSAVVQLYAADRNCMWSKKCSGVACLVKDNPQRSYFLRIFDIKDGKLLWEQELYNNFVYNSPRGYFHTFAGDTCQVALNFANEEEAKKFRKAVTDLLGRRQRKSEKRRDPPNGPNLPMATVDIKNPEITTNRFYGPQVNNISHTKEKKKGKAKKKRLTKADIGTPSNFQHIGHVGWDPNTGFDLNNLDPELKNLFDMCGISEAQLKDRETSKVIYDFIEKTGGVEAVKNELRRQAPPPPPPSRGGPPPPPPPPHNSGPPPPPARGRGAPPPPPSRAPTAAPPPPPPSRPSVAVPPPPPNRMYPPPPPALPSSAPSGPPPPPPSVLGVGPVAPPPPPPPPPPPGPPPPPGLPSDGDHQVPTTAGNKAALLDQIREGAQLKKVEQNSRPVSCSGRDALLDQIRQGIQLKSVADGQESTPPTPAPTSGIVGALMEVMQKRSKAIHSSDEDEDEDDEEDFEDDDEWED

SEQ ID NO: 3 - DNA, 3074 bp, Homo sapiens
attgtcttccaggaaacagctccctcagtttggaatcagctctcccgctgcggccgcagtagccggagccggagccgcagccaccggtgccttcctttcccgccgccgcccagccgccgtccggcctccctcgggcccgagcgcagaccaggctccagccgcgcggcgccggcagcctcgcgctccctctcgggtctctctcgggcctcgggcaccgcgtcctgtggggcggccgcctgcctgcccgcccgcccgcagccccttcgctgcgcggcccctgggcggccgctgccatgggcaccgacagccgcgcggccaaggcgctcctggcgcgggcccgcaccctgcacctgcagacggggaacctgctgaactggggccgcctgcggaagaagtgcccgtccacgcacagcgaggagcttcatgattgtatccaaaaaaccttgaatgaatggagttcccaaatcaacccagatttggtcagggagtttccagatgtcttggaatgcactgtatctcatgcagtagaaaagataaatcctgatgaaagagaagaaatgaaagtttctgcaaaactgttcattgtagaatcaaactcttcatcatcaactagaagtgcagttgacatggcctgttcagtccttggagttgcacagctggattctgtgatcattgcttcacctcctattgaagatggagttaatctttccttggagcatttacagccttactgggaggaattagaaaacttagttcagagcaaaaagattgttgccataggtacctctgatctagacaaaacacagttggaacagctgtatcagtgggcacaggtaaaaccaaatagtaaccaagttaatcttgcctcctgctgtgtgatgccaccagatttgactgcatttgctaaacaatttgacatacagctgttgactcacaatgatccaaaagaactgctttctgaagcaagtttccaagaagctcttcaggaaagcattcctgacattcaagcgcacgagtgggtgccgctgtggctactgcggtattcggtcattgtgaaaagtagaggaattatcaaatcaaaaggctacattttacaagctaaaagaaggggttcttaactgacttaggagcataacttacctgtaatttccttcaatatgagagaaaattgagatgtgtaaaaatctagttactgcctgtaaatggtgtcattgaggcagatattctttcgtcatatttgacagtatgttgtctgtcaagttttaaatacttatcttgcctccatatcaatccattctcatgaacctctgtattgctttccttaaactattgttttctaattgaaattgtctataaagaaaatacttgcaatatatttttcctttatttttatgactaatataaatcaagaaaatttgttgttagatatattttggcctaggtatcagggtaatgtatatacatattttttatttccaaaaaaaattcattaattgcttcttaactcttattataaccaagcaatttaattacaattgttaaaactgaaatactggaagaagatatttttcctgtcattgatgagatatatcagagtaactggagtagctgggatttactagtagtgtaaataaaattcactcttcaatacatgaatggaaacttaaatttttttttatgtgtccttgcttatagtttagctgtaataatttaaccttgtattcttgtgccatattctgtctttttattacttataaagacaaaccaaagtaaatctgaaaggagactagaagctttgaaattattgtttgggggttttataaaagcaactactgtcacctccatccagattcttttaaattattgatccatccatagtatatattgctactcattcaagaatcctcaataagtattgagtatttaccatatgttgggatactgtgggctctggagagaggagggggcaatagagctaggaattaagaatcagttgagtaaaatgtgtaatatttattccccattaataactgactaggaaggactaaaagccagaaaggggatgaaaaaaaaatccttaattcagggccgacattatctacttaaacaactttgagatatggtcttaattattttaaagcagaataatataattgaaagtttatagctaaaagagactatataggtcatttagtataattcttcattagtttacgaaccacaaaattgcaaataaataagctatgaactttgatgtacactataaatctccttaattctataaatttgtgtctgtaacctgaatagtttgaaaacttctttaaaaatctcttgtatttcatccgggcgcagtggctcacacctgtaatcccagcactttgggaggccgaggtgggcagatcacgaggtcaggagtttgagaccagcctgaccaacatggtaaaaccccatctctactaaaatacaaaaattggctgggcgtggtggcactcgcctgtaatctcagctacttgggaggctgaggcaggagaatcgcttgaacccgggaggcggaggttacagtgagccgagatcacatcactgcactccagcctgggcgacagagcgagactccatctcaaaaaaaaaaaaaaactcttgtatctcaatatttttaaaccacaggcctaaataaaactaattttgctcaagttttctcaacctagggaaaaagaactatggttccatattcaaaataaatattatagacccttttcctaagtaggattttgtggtttactgattgggtaatttgatcattaaaattatgtgaaatctgcccgggcacacctcatgcctgtaatcccagcactctgggaggccaaggcagatgatcacctgaggtcaggagttctagaccagcctggctaacatggtgaaaccctgtatctgctaaaaatacaaaaattagccaggcgtggtggcgggctcctgtaatcccagctactttggaggcgaggcacgagaatcgcttgaacctgggaggcggagtttgcagtgagccgagatcacgccattgcactccagcctgggcg

SEQ ID NO: 4 - protein, 274 aa, Homo sapiens
MGTDSRAAKALLARARTLHLQTGNLLNWGRLRKKCPSTHSEELHDCIQKTLNEWSSQINPDLVREFPDVLECTVSHAVEKINPDEREEMKVSAKLFIVESNSSSSTRSAVDMACSVLGVAQLDSVIIASPPIEDGVNLSLEHLQPYWEELENLVQSKKIVAIGTSDLDKTQLEQLYQWAQVKPNSNQVNLASCCVMPPDLTAFAKQFDIQLLTHNDPKELLSEASFQEALQESIPDIQAHEWVPLWLLRYSVIVKSRGIIKSKGYILQAKRRGS

SEQ ID NO: 5 - DNA, 818 bp, Homo sapiens
cgcagcgggtcctctctatctagctccagcctctcgcctgcgccccactccccgcgtcccgcgtcctagccgaccatggccgggcccctgcgcgccccgctgctcctgctggccatcctggccgtggccctggccgtgagccccgcggccggctccagtcccggcaagccgccgcgcctggtgggaggccccatggacgccagcgtggaggaggagggtgtgcggcgtgcactggactttgccgtcggcgagtacaacaaagccagcaacgacatgtaccacagccgcgcgctgcaggtggtgcgcgcccgcaagcagatcgtagctggggtgaactacttcttggacgtggagctgggccgaaccacgtgtaccaagacccagcccaacttggacaactgccccttccatgaccagccacatctgaaaaggaaagcattctgctctttccagatctacgctgtgccttggcagggcacaatgaccttgtcgaaatccacctgtcaggacgcctaggggtctgtaccgggctggcctgtgcctatcacctcttatgcacacctcccaccccctgtattcccacccctggactggtggcccctgccttggggaaggtctccccatgtgcctgcaccaggagacagacagagaaggcagcaggcggcctttgttgctcagcaaggggctctgccctccctccttccttcttgcttctcatagccccggtgtgcggtgcatacacccccacctcctgcaataaaatagtagcatcggcaaaaaaaaaaaaaaaaaaaaaaaaaaaaaaaaaaaaaaaaaaa

SEQ ID NO: 6 - protein, 146 aa, Homo sapiens
MAGPLRAPLLLLAILAVALAVSPAAGSSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 7 - DNA, 5623 bp, Homo sapiens
agccagcggacgtccaggaaccgggatgcctccagcagtgaggcggtcagcctgcagcatgggatggctgtggatctttggggcagccctggggcagtgtctgggctacagttcacagcagcaaagggtgccatttcttcagcctcccggtcaaagtcaactgcaagcgagttatgtggagtttagacccagccagggttgtagccctggatactatcgggatcataaaggcttgtataccggacggtgtgttccctgcaattgcaacggacattcaaatcaatgccaggatggctcaggcatatgtgttaactgtcagcacaacaccgcgggagagcactgtgaacgctgccaggagggctactatggcaacgccgtccacggatcctgcagggcctgcccatgtcctcacactaacagctttgccactggctgtgtggtgaatgggggagacgtgcggtgctcctgcaaagctgggtacacaggaacacagtgtgaaaggtgtgcaccgggatatttcgggaatccccagaaattcggaggtagctgccaaccatgcagttgtaacagcaatggccagctgggcagctgtcatcccctgactggagactgcataaaccaagaacccaaagatagcagccctgcagaagaatgtgatgattgcgacagctgtgtgatgaccctcctgaacgacctggccaccatgggcgagcagctccgcctggtcaagtctcagctgcagggcctgagtgccagcgcagggcttctggagcagatgaggcacatggagacccaggccaaggacctgaggaatcagttgctcaactaccgttctgccatttcaaatcatggatcaaaaatagaaggcctggaaagagaactgactgatttgaatcaagaatttgagactttgcaagaaaaggctcaagtaaattccagaaaagcacaaacattaaacaacaatgttaatcgggcaacacaaagcgcaaaagaactggatgtgaagattaaaaatgtcatccggaatgtgcacattcttttaaagcagatctctgggacagatggagagggaaacaacgtgccttcaggtgacttttccagagagtgggctgaagcccagcgcatgatgagggaactgcggaacaggaactttggaaagcacctcagagaagcagaagctgataaaagggagtcgcagctcttgctgaaccggataaggacctggcagaaaacccaccagggggagaacaatgggcttgctaacagtatccgggattctttaaatgaatacgaagccaaactcagtgaccttcgtgctcggctgcaggaggcagctgcccaagccaagcaggcaaatggcttgaaccaagaaaacgagagagctttgggagccattcagagacaagtgaaagaaataaattccctgcagagtgatttcaccaagtatctaaccactgcagactcatctttgttgcaaaccaacattgcgctgcagctgatggagaaaagccagaaggaatatgaaaaattagctgccagtttaaatgaagcaagacaagaactaagtgacaaagtaagagaactttccagatctgctggcaaaacatcccttgtggaggaggcagaaaagcacgcgcggtccttacaagagctggcaaagcagctggaagagatcaagagaaacgccagcggggatgagctggtgcgctgtgctgtggatgccgccaccgcctacgagaacatcctcaatgccatcaaagcggccgaggacgcagccaacagggctgccagtgcatctgaatctgccctccagacagtgataaaggaagatctgccaagaaaagctaaaaccctgagttccaacagtgataaactgttaaatgaagccaagatgacacaaaagaagctaaagcaagaagtcagtccagctctcaacaacctacagcaaaccctgaatattgtgacagttcagaaagaagtgatagacaccaatctcacaactctccgagatggtcttcatgggatacagagaggtgatattgatgctatgatcagtagtgcaaagagcatggtcagaaaggccaacgacatcacagatgaggttctggatgggctcaaccccatccagacagatgtggaaagaattaaggacacctatgggaggacacagaacgaagacttcaaaaaggctctgactgatgcagataactcggtgaataagttaaccaacaaactacctgatctttggcgcaagattgaaagtatcaaccaacagctgttgcccttgggaaacatctctgacaacatggacagaatacgagaactaattcagcaggccagagatgctgccagtaaggttgctgtccccatgaggttcaatggtaaatctggagtcgaagtccgactgccaaatgacctggaagatttgaaaggatatacatctctgtccttgtttctccaaaggcccaactcaagagaaaatgggggtactgagaatatgtttgtgatgtaccttggaaataaagatgcctcccgggactacatcggcatggcagttgtggatggccagctcacctgtgtctacaacctgggggaccgtgaggctgaactccaagtggaccagatcttgaccaagagtgagactaaggaggcagttatggatcgggtgaaatttcagagaatttatcagtttgcaaggcttaattacaccaaaggagccacatccagtaaaccagaaacacccggagtctatgacatggatggtagaaatagcaatacactccttaatttggatcctgaaaatgttgtattttatgttggaggttacccacctgattttaaacttcccagtcgactaagtttccctccatacaaaggttgtattgaattagatgacctcaatgaaaatgttctgagcttgtacaacttcaaaaaaacattcaatctcaacacaactgaagtggagccttgtagaaggaggaaggaagagtcagacaaaaattattttgaaggtacgggctatgctcgagttccaactcaaccacatgctcccatcccaacctttggacagacaattcagaccaccgtggatagaggcttgctgttctttgcagaaaacggggatcgcttcatatctctaaatatagaagatggcaagctcatggtgagatacaaactgaattcagagctaccaaaagagagaggagttggagacgccataaacaacggcagagaccattcgattcagatcaaaattggaaaactccaaaagcgtatgtggataaatgtggacgttcaaaacactataattgatggtgaagtatttgatttcagcacatattatctgggaggaattccaattgcaatcagggaaagatttaacatttctacgcctgctttccgaggctgcatgaaaaatttgaagaaaaccagtggtgtcgttagattgaatgatactgtgggagtaaccaaaaagtgctcggaagactggaagcttgtgcgatctgcctcattctccagaggaggacaattgagtttcactgatttgggcttaccacctactgaccacctccaggcctcatttggatttcagacctttcaacccagtggcatattattagatcatcagacatggacaaggaacctgcaggtcactctggaagatggttacattgaattgagcaccagcgatagcggcagcccaatttttaaatctccacagacgtatatggatggtttactgcattatgtatctgtaataagcgacaactctggactacggcttctcatcgatgaccagcttctgagaaatagcaaaaggctaaaacacatttcaagttcccggcagtctctgcgtctgggcgggagcaattttgagggttgtattagcaatgtttttgtccagaggttatcactgagtcctgaagtcctagatttgaccagtaactctctcaagagagatgtgtccctgggaggctgcagtttaaacaaaccaccttttctaatgttgcttaaaggttctaccaggtttaacaagaccaagacttttcgtatcaaccagctgttgcaggacacaccagtggcctccccaaggagcgtgaaggtgtggcaagatgcttgctcaccacttcccaagacccaggccaatcatggagccctccagtttggggacattcccaccagccacttgctattcaagcttcctcaggagctgctgaaacccaggtcacagtttgctgtggacatgcagacaacatcctccagaggactggtgtttcacacgggcactaagaactcctttatggctctttatctttcaaaaggacgtctggtctttgcactggggacagatgggaaaaaattgaggatcaaaagcaaggagaaatgcaatgatgggaaatggcacacggtggtgtttggccatgatggggaaaaggggcgcttggttgtggatggactgagggcccgggagggaagtttgcctggaaactccaccatcagcatcagagcgccagtttacctgggatcacctccatcagggaaaccaaagagcctccccacaaacagctttgtgggatgcctgaagaactttcagctggattcaaaacccttgtataccccttcttcaagcttcggggtgtcttcctgcttgggtggtcctttggagaaaggcatttatttctctgaagaaggaggtcatgtcgtcttggctcactctgtattgttggggccagaatttaagcttgttttcagcatccgcccaagaagtctcactgggatcctaatacacatcggaagtcagcccgggaagcacttatgtgtttacctggaggcaggaaaggtcacggcctctatggacagtggggcaggtgggacctcaacgtcggtcacaccaaagcagtctctgtgtgatggacagtggcactcggtggcagtcaccataaaacaacacatcctgcacctggaactggacacagacagtagctacacagctggacagatccccttcccacctgccagcactcaagagccactacaccttggaggtgctccagccaatttgacgacactgaggatccctgtgtggaaatcattctttggctgtctgaggaatattcatgtcaatcacatccctgtccctgtcactgaagccttggaagtccaggggcctgtcagtctgaatggttgtcctgaccagtaacccaagcctatttcacagcaaggaaattcaccttcaaaagcactgattacccaatgcacctccctccccagctcgagatcattcttcactcaggacacaaaccagacaggtttaatagcgaatctaattttgaattctgaccatggatacccatcactttggcattcagtgctacatgtgtattttatataaaaatcccatttcttgaagataaaaaaattgttattcaaattgttatgcacagaatgtttttggtaatattaatttccactaaaaaattaaatgtcttttaagaaacattcttttccacttgttaaaaaaattaaatatattttaaagcactttaagaatatgaaactttcatatatgttaaaggattataatttatggaattaaaaaatgcagtgtagtccttaaaaaaaa

SEQ ID NO: 8 - protein, 1724 aa, Homo sapiens
MPPAVRRSACSMGWLWIFGAALGQCLGYSSQQQRVPFLQPPGQSQLQASYVEFRPSQGCSPGYYRDHKGLYTGRCVPCNCNGHSNQCQDGSGICVNCQHNTAGEHCERCQEGYYGNAVHGSCRACPCPHTNSFATGCVVNGGDVRCSCKAGYTGTQCERCAPGYFGNPQKFGGSCQPCSCNSNGQLGSCHPLTGDCINQEPKDSSPAEECDDCDSCVMTLLNDLATMGEQLRLVKSQLQGLSASAGLLEQMRHMETQAKDLRNQLLNYRSAISNHGSKIEGLERELTDLNQEFETLQEKAQVNSRKAQTLNNNVNRATQSAKELDVKIKNVIRNVHILLKQISGTDGEGNNVPSGDFSREWAEAQRMMRELRNRNFGKHLREAEADKRESQLLLNRIRTWQKTHQGENNGLANSIRDSLNEYEAKLSDLRARLQEAAAQAKQANGLNQENERALGAIQRQVKEINSLQSDFTKYLTTADSSLLQTNIALQLMEKSQKEYEKLAASLNEARQELSDKVRELSRSAGKTSLVEEAEKHARSLQELAKQLEEIKRNASGDELVRCAVDAATAYENILNAIKAAEDAANRAASASESALQTVIKEDLPRKAKTLSSNSDKLLNEAKMTQKKLKQEVSPALNNLQQTLNIVTVQKEVIDTNLTTLRDGLHGIQRGDIDAMISSAKSMVRKANDITDEVLDGLNPIQTDVERIKDTYGRTQNEDFKKALTDADNSVNKLTNKLPDLWRKIESINQQLLPLGNISDNMDRIRELIQQARDAASKVAVPMRFNGKSGVEVRLPNDLEDLKGYTSLSLFLQRPNSRENGGTENMFVMYLGNKDASRDYIGMAVVDGQLTCVYNLGDREAELQVDQILTKSETKEAVMDRVKFQRIYQFARLNYTKGATSSKPETPGVYDMDGRNSNTLLNLDPENVVFYVGGYPPDFKLPSRLSFPPYKGCIELDDLNENVLSLYNFKKTFNLNTTEVEPCRRRKEESDKNYFEGTGYARVPTQPHAPIPTFGQTIQTTVDRGLLFFAENGDRFISLNIEDGKLMVRYKLNSELPKERGVGDAINNGRDHSIQIKIGKLQKRMWINVDVQNTIIDGEVFDFSTYYLGGIPIAIRERFNISTPAFRGCMKNLKKTSGVVRLNDTVGVTKKCSEDWKLVRSASFSRGGQLSFTDLGLPPTDHLQASFGFQTFQPSGILLDHQTWTRNLQVTLEDGYIELSTSDSGSPIFKSPQTYMDGLLHYVSVISDNSGLRLLIDDQLLRNSKRLKHISSSRQSLRLGGSNFEGCISNVFVQRLSLSPEVLDLTSNSLKRDVSLGGCSLNKPPFLMLLKGSTRFNKTKTFRINQLLQDTPVASPRSVKVWQDACSPLPKTQANHGALQFGDIPTSHLLFKLPQELLKPRSQFAVDMQTTSSRGLVFHTGTKNSFMALYLSKGRLVFALGTDGKKLRIKSKEKCNDGKWHTVVFGHDGEKGRLVVDGLRAREGSLPGNSTISIRAPVYLGSPPSGKPKSLPTNSFVGCLKNFQLDSKPLYTPSSSFGVSSCLGGPLEKGIYFSEEGGHVVLAHSVLLGPEFKLVFSIRPRSLTGILIHIGSQPGKHLCVYLEAGKVTASMDSGAGGTSTSVTPKQSLCDGQWHSVAVTIKQHILHLELDTDSSYTAGQIPFPPASTQEPLHLGGAPANLTTLRIPVWKSFFGCLRNIHVNHIPVPVTEALEVQGPVSLNGCPDQ

SEQ ID NO: 9 - DNA, 2825 bp, Homo sapiens
gtacggcttccggtggcgggacgcggggccgcgcacgcgggaaaagcttccccggtgtccccccatccccctccccgcgccccccccgcgtccccccagcgcgcccacctctcgcgccggggccctcgcgaggccgcagcctgaggagattcccaacctgctgagcatccgcacacccactcaggagttggggcccagctcccagtttacttggtttcccttgtgcagcctggggctctgcccaggccaccacaggcaggggtcgacatggcagagacactggagttcaacgacgtctatcaggaggtgaaaggttccatgaatgatggtcgactgaggttgagccgtcagggcatcatcttcaagaatagcaagacaggcaaagtggacaacatccaggctggggagttaacagaaggtatctggcgccgtgttgctctgggccatggacttaaactgcttacaaagaatggccatgtctacaagtatgatggcttccgagaatcggagtttgagaaactctctgatttcttcaaaactcactatcgccttgagctaatggagaaggacctttgtgtgaagggctggaactgggggacagtgaaatttggtgggcagctgctttcctttgacattggtgaccagccagtctttgagatacccctcagcaatgtgtcccagtgcaccacaggcaagaatgaggtgacactggaattccaccaaaacgatgacgcagaggtgtctctcatggaggtgcgcttctacgtcccacccacccaggaggatggtgtggaccctgttgaggcctttgcccagaatgtgttgtcaaaggcggatgtaatccaggccacgggagatgccatctgcatcttccgggagctgcagtgtctgactcctcgtggtcgttatgacattcggatctaccccacctttctgcacctgcatggcaagacctttgactacaagatcccctacaccacagtactgcgtctgtttttgttaccccacaaggaccagcgccagatgttctttgtgatcagcctggatcccccaatcaagcaaggccaaactcgctaccacttcctgatcctcctcttctccaaggacgaggacatttcgttgactctgaacatgaacgaggaagaagtggagaagcgctttgagggtcggctcaccaagaacatgtcaggatccctctatgagatggtcagccgggtcatgaaagcactggtaaaccgcaagatcacagtgccaggcaacttccaagggcactcaggggcccagtgcattacctgttcctacaaggcaagctcaggactgctctacccgctggagcggggcttcatctacgtccacaagccacctgtgcacatccgcttcgatgagatctcctttgtcaactttgctcgtggtaccactactactcgttcctttgactttgaaattgagaccaagcagggcactcagtataccttcagcagcattgagagggaggagtacgggaaactgtttgattttgtcaacgcgaaaaagctcaacatcaaaaaccgaggattgaaagagggcatgaacccaagctacgatgaatatgctgactctgatgaggaccagcatgatgcctacttggagaggatgaaggaggaaggcaagatccgggaggagaatgccaatgacagcagcgatgactcaggagaagaaaccgatgagtcattcaacccaggtgaagaggaggaagatgtggcagaggagtttgacagcaacgcctctgccagctcctccagtaatgagggtgacagtgaccgggatgagaagaagcggaaacagctcaaaaaggccaagatggccaaggaccgcaagagccgcaagaagcctgtggaggtgaagaagggcaaagaccccaatgcccccaagaggcccatgtctgcatacatgctgtggctcaatgccagccgagagaagatcaagtcagaccatcctggcatcagcatcacggatctttccaagaaggcaggcgagatctggaagggaatgtccaaagagaagaaagaggagtgggatcgcaaggctgaggatgccaggagggactatgaaaaagccatgaaagaatatgaagggggccgaggcgagtcttctaagagggacaagtcaaagaagaagaagaaagtaaaggtaaagatggaaaagaaatccacgccctctaggggctcatcatccaagtcgtcctcaaggcagctaagcgagagcttcaagagcaaagagtttgtgtctagtgatgagagctcttcgggagagaacaagagcaaaaagaagaggaggaggagcgaggactctgaagaagaagaactagccagtactccccccagctcagaggactcagcgtcaggatccgatgagtagaaacggaggaaggttctctttgcgcttgccttctcacaccccccgactccccacccatattttggtaccagtttctcctcatgaaatgcagtccctggattctgtgccatctgaacatgctctcctgttggtgtgtatgtcactagggcagtggggagacgtcttaactctgctgcttcccaaggatggctgtttataatttggggagagatagggtgggaggcagggcaatgcaggatccaaatcctcatcttactttcccgaccttaaggatgtagctgctgcttgtcctgttcaagttgctggagcaggggtcatgtgaggccaggcctgtagctcctacctggggcctatttctactttcattttgtatttctggtctgtgaaaatgatttaataaagggaactgactttggaaaccaaaaaaa

SEQ ID NO: 10 - protein, 709 aa, Homo sapiens
MAETLEFNDVYQEVKGSMNDGRLRLSRQGIIFKNSKTGKVDNIQAGELTEGIWRRVALGHGLKLLTKNGHVYKYDGFRESEFEKLSDFFKTHYRLELMEKDLCVKGWNWGTVKFGGQLLSFDIGDQPVFEIPLSNVSQCTTGKNEVTLEFHQNDDAEVSLMEVRFYVPPTQEDGVDPVEAFAQNVLSKADVIQATGDAICIFRELQCLTPRGRYDIRIYPTFLHLHGKTFDYKIPYTTVLRLFLLPHKDQRQMFFVISLDPPIKQGQTRYHFLILLFSKDEDISLTLNMNEEEVEKRFEGRLTKNMSGSLYEMVSRVMKALVNRKITVPGNFQGHSGAQCITCSYKASSGLLYPLERGFIYVHKPPVHIRFDEISFVNFARGTTTTRSFDFEIETKQGTQYTFSSIEREEYGKLFDFVNAKKLNIKNRGLKEGMNPSYDEYADSDEDQHDAYLERMKEEGKIREENANDSSDDSGEETDESFNPGEEEEDVAEEFDSNASASSSSNEGDSDRDEKKRKQLKKAKMAKDRKSRKKPVEVKKGKDPNAPKRPMSAYMLWLNASREKIKSDHPGISITDLSKKAGEIWKGMSKEKKEEWDRKAEDARRDYEKAMKEYEGGRGESSKRDKSKKKKKVKVKMEKKSTPSRGSSSKSSSRQLSESFKSKEFVSSDESSSGENKSKKKRRRSEDSEEEELASTPPSSEDSASGSDE

SEQ ID NO: 11 - DNA, 621 bp, Homo sapiens
cggaggtttgcgcgccttggtgagccgttggcgtggtggtcccggagtgatcctggcagccggtggggaagacaaggagggtttgagcatggcagaaggaaacaccctgatatcagtggattatgaaatttttgggaaggtgcaaggggtgtttttccgtaagcatactcaggctgagggtaaaaagctgggattggtaggctgggtccagaacactgaccggggcacagtgcaaggacaattgcaaggtcccatctccaaggtgcgtcatatgcaggaatggcttgaaacaagaggaagtcctaaatcacacatcgacaaagcaaacttcaacaatgaaaaagtcatcttgaagttggattactcagacttccaaattgtaaaataatggcctgaatttaagttttctaagataaactcagtggtttggtttttattattaatagagatagaactattgtgtgttaatattagcattagtcaataagttattttaatgtcagatttttgaatgttattatatattacctgtatgatggaaggattaccactgtacacaaatctaatcaataaaaacgttagaaccttctgcttagagtacttttaaaaaaaaaaaaaaaa

SEQ ID NO: 12 - protein, 99 aa, Homo sapiens
MAEGNTLISVDYEIFGKVQGVFFRKHTQAEGKKLGLVGWVQNTDRGTVQGQLQGPISKVRHMQEWLETRGSPKSHIDKANFNNEKVILKLDYSDFQIVK

SEQ ID NO: 13 - DNA, 5414 bp, Homo sapiens
ttgagaaacgaggtctagggttggcaggcaatacaatacaactcttgatgcctctcattcctctgtcaggctccacaaggacactttaaggactaatttgatttaaatcaattcattaaaaaatatctatagcttttagctagaagaaccccaaggagtgattattttgcaatagggctgagttgggccgggcatggtggctcacacctgtaatcccagcactttgggggccgcgcgtcggagtttccccctttctagggtgaggatggttctacacagccacccggagttccttagttgaaaggtgcgccctgctgtgacagcatggacaccacgttgctgaaaacatgctttgggactgccactgaatttatcttttgcggttttatgacaaagttattagtagtttcccttttttgaattagtattttgaagttaatatcacaatgagttcaggcttatggagccaagaaaaagtcacttcaccctactgggaagagcggattttttacttgcttcttcaagaatgcagcgttacagacaaacaaacacaaaagctccttaaagtaccgaagggaagtataggacagtatattcaagatcgttctgtggggcattcaaggattccttctgcaaaaggcaagaaaaatcagattggattaaaaattctagagcaacctcatgcagttctctttgttgatgaaaaggatgttgtagagataaatgaaaagttcacagagttacttttggcaattaccaattgtgaggagaggttcagcctgtttaaaaacagaaacagactaagtaaaggcctccaaatagacgtgggctgtcctgtgaaagtacagctgagatctggggaagaaaaatttcctggagttgtacgcttcagaggacccctgttagcagagaggacagtctccggaatattctttggagttgaattgctggaagaaggtcgtggtcaaggtttcactgacggggtgtaccaagggaaacagctttttcagtgtgatgaagattgtggcgtgtttgttgcattggacaagctagaactcatagaagatgatgacactgcattggaaagtgattacgcaggtcctggggacacaatgcaggtcgaacttcctcctttggaaataaactccagagtttctttgaaggttggagaaacaatagaatctggaacagttatattctgtgatgttttgccaggaaaagaaagcttaggatattttgttggtgtggacatggataaccctattggcaactgggatggaagatttgatggagtgcagctttgtagttttgcgtgtgttgaaagtacaattctattgcacatcaatgatatcatcccagagagtgtgacgcaggaaaggaggcctcccaaacttgcctttatgtcaagaggtgttggggacaaaggttcatccagtcataataaaccaaaggctacaggatctacctcagaccctggaaatagaaacagatctgaattattttataccttaaatgggtcttctgttgactcacaaccacaatccaaatcaaaaaatacatggtacattgatgaagttgcagaagaccctgcaaaatctcttacagagatatctacagactttgaccgttcttcaccaccactccagcctcctcctgtgaactcactgaccaccgagaacagattccactctttaccattcagtctcaccaagatgcccaataccaatggaagtattggccacagtccactttctctgtcagcccagtctgtaatggaagagctaaacactgcacccgtccaagagagtccacccttggccatgcctcctgggaactcacatggtctagaagtgggctcattggctgaagttaaggagaaccctcctttctatggggtaatccgttggatcggtcagccaccaggactgaatgaagtgctcgctggactggaactggaagatgagtgtgcaggctgtacggatggaaccttcagaggcactcggtatttcacctgtgccctgaagaaggcgctgtttgtgaaactgaagagctgcaggcctgactctaggtttgcatcattgcagccggtttccaatcagattgagcgctgtaactctttagcatttggaggctacttaagtgaagtagtagaagaaaatactccaccaaaaatggaaaaagaaggcttggagataatgattgggaagaagaaaggcatccagggtcattacaattcttgttacttagactcaaccttattctgcttatttgcttttagttctgttctggacactgtgttacttagacccaaagaaaagaacgatgtagaatattatagtgaaacccaagagctactgaggacagaaattgttaatcctctgagaatatatggatatgtgtgtgccacaaaaattatgaaactgaggaaaatacttgaaaaggtggaggctgcatcaggatttacctctgaagaaaaagatcctgaggaattcttgaatattctgtttcatcatattttaagggtagaacctttgctaaaaataagatcagcaggtcaaaaggtacaagattgttacttctatcaaatttttatggaaaaaaatgagaaagttggcgttcccacaattcagcagttgttagaatggtcttttatcaacagtaacctgaaatttgcagaggcaccatcatgtctgattattcagatgcctcgatttggaaaagactttaaactatttaaaaaaatttttccttctctggaattaaatataacagatttacttgaagacactcccagacagtgccggatatgtggagggcttgcaatgtatgagtgtagagaatgctacgacgatccggacatctcagctggaaaaatcaagcagttttgtaaaacctgcaacactcaagtccaccttcatccgaagaggctgaatcataaatataacccagtgtcacttcccaaagacttacccgactgggactggagacacggctgcatcccttgccagaatatggagttatttgctgttctctgcatagaaacaagccactatgttgcttttgtgaagtatgggaaggacgattctgcctggctcttctttgacagcatggccgatcgggatggtggtcagaatggcttcaacattcctcaagtcaccccatgcccagaagtaggagagtacttgaagatgtctctggaagacctgcattccttggactccaggagaatccaaggctgtgcacgaagactgctttgtgatgcatatatgtgcatgtaccagagtccaacaatgagtttgtacaaataactggggtcatcgggaaaggcaaagaaactgaaggcagagtcctaacgttgcatcttattcgagctggcagttctgttcacgtccattgccggcaatggatgtctttgtggtgatgatccttcagaaaaggatgcctctgtttaaaaacaaattgcttttgtgtccctgaagtatttaataagaagcattttgcactctagaaagtatgtttgtgttggttttttaagaagtctaaatgaagttattaatacctgaagctttaagttaagtgcattgatcatatgatatttttggaagcatacaattttaattgtggaagtttaaagcctcttttagtccattgagaatgtaaataaatgtgtcttctttatggaccaaggatatgaaatcatttttcttttgtagctaacggttgccttgaggaagaaataatttggttttattaagagtctactctcaatccagttattagagatgtactgagtttgatttgttaatcctttctatatactgctgatcttgcatgtctacaatctgctcagtttttctgtgtttctgcaatagtggtcagaaaaatacttaaattcccttaatggtgttgttttctatttgttctggttttgagataaatgagtgattctgtccccaaatgtccatttttgaagtgattttcctggaggattagggtatttagcagttgaagctcttcattcatagtagttactgtcagctaacaggttttttaaggcttttaactattaatattttatggaatggggcaaagtaaattgatgaaagaattggagtgataatagtcctttacaaacatacagtccataagaaaatgaatttggcatatagaattattacaatttcctgggagagatggatatttaaacctctattattttagacaagactgtctagaacttaagtttgatctgtcagccagtactcccattaaattcagtgtagtttcacttgatagaatcagatatgttatcgaaatgttagcagcagcttcatcctccttctgattaaagtaagtagaaatgggatgttttgtttaataacagccatagtgtgtgtttagaccacagcggatgttgtagaccaggaccatagatgatacatgtcagtgctgtggaatgtgcattctctgagtgttgttttgtggtatcattgtctttcctgaatgactttctaactgtgcagaaaggcagaaaagtcatcatatgtatatgtcatatgactttataaaatatttaatgtgacaaaaagtggaaagaatctttacaaaccctgcaattacttttttaaaggcacttttactctttggttttatcattccattttgctaatatttactagctttataaattacagtaaggtacaaaaactcatcttgtaatattttcatttttgaagtgaaaaagtacatatattttgcacaaggttttatactgctaagtgcttggttggggtggtgagatgatgattagatcaggggtgaggctgagagactctgggtttagggctagccctgcctccatctcccttgggtaaaatgaagggtgtggggtaaaagatgcataaggccttttctagctctgacagcctagaagtccaatcaccctgtaataaatatgtgttgaatgaagaaatgggtgaatgagcttgtcaatgtgattttaaaaaattgactacctggaggaatgattaggaatctaaatgaagccagccctcggtatctgcaggtttctcatccatggattcaaccaactgcaaatggaaaatacgattttttttaaaaaaaggatggttacatccgtattgaacatgtacagacttttttcttgtcattattctctgaacaatacaagaactctttatgtagcatttacatttattaggtattataagtaatctagagattatttaattaaaatatacaggaggatgtgtgtcagttatatgcaaattctgtaccattttgtatcagggaattgagcatcttcagatgttggtatctgcagggatcctggaaccaaacccctgcagatactaagggctgacgatctaggtaagactggatttaacagttgg

SEQ ID NO: 14 - protein, 960 aa, Homo sapiens
YFEVNITMSSGLWSQEKVTSPYWEERIFYLLLQECSVTDKQTQKLLKVPKGSIGQYIQDRSVGHSRIPSAKGKKNQIGLKILEQPHAVLFVDEKDVVEINEKFTELLLAITNCEERFSLFKNRNRLSKGLQIDVGCPVKVQLRSGEEKFPGVVRFRGPLLAERTVSGIFFGVELLEEGRGQGFTDGVYQGKQLFQCDEDCGVFVALDKLELIEDDDTALESDYAGPGDTMQVELPPLEINSRVSLKVGETIESGTVIFCDVLPGKESLGYFVGVDMDNPIGNWDGRFDGVQLCSFACVESTILLHINDIIPESVTQERRPPKLAFMSRGVGDKGSSSHNKPKATGSTSDPGNRNRSELFYTLNGSSVDSQPQSKSKNTWYIDEVAEDPAKSLTEISTDFDRSSPPLQPPPVNSLTTENRFHSLPFSLTKMPNTNGSIGHSPLSLSAQSVMEELNTAPVQESPPLAMPPGNSHGLEVGSLAEVKENPPFYGVIRWIGQPPGLNEVLAGLELEDECAGCTDGTFRGTRYFTCALKKALFVKLKSCRPDSRFASLQPVSNQIERCNSLAFGGYLSEVVEENTPPKMEKEGLEIMIGKKKGIQGHYNSCYLDSTLFCLFAFSSVLDTVLLRPKEKNDVEYYSETQELLRTEIVNPLRIYGYVCATKIMKLRKILEKVEAASGFTSEEKDPEEFLNILFHHILRVEPLLKIRSAGQKVQDCYFYQIFMEKNEKVGVPTIQQLLEWSFINSNLKFAEAPSCLIIQMPRFGKDFKLFKKIFPSLELNITDLLEDTPRQCRICGGLAMYECRECYDDPDISAGKIKQFCKTCNTQVHLHPKRLNHKYNPVSLPKDLPDWDWRHGCIPCQNMELFAVLCIETSHYVAFVKYGKDDSAWLFFDSMADRDGGQNGFNIPQVTPCPEVGEYLKMSLEDLHSLDSRRIQGCARRLLCDAYMCMYQSPTMSLYK

SEQ ID NO: 15 - DNA, 1300 bp, Homo sapiens
gccgccggcccagtgaggctgggttcgaggagctggagcgggaaactggagcttaaattctggcggcgagatggacattctgaaatcagagatccttcggaagcggcagctggtggaggacaggaacctgctggtggaaaataaaaaatatttcaagcgtagtgagctcgccaaaaaagaagaggaagcatattttgaaagatgtggctacaagatacagccaaaagaggaggaccagaaaccattaacttcatcgaatccagtgttagaacttgaactggcagaggaaaaattacctatgacgctttctaggcaagaggtcatcagaagattgagagaaagaggagaaccaatcagactatttggagagactgattatgatgcttttcaacgtttaaggaaaatagagatcctcacaccagaagttaacaagggattgaggaatgatttgaaagcagccttggataagattgatcagcagtacctcaatgaaatcgtcggcggtcaggagcctggagaggaagacacacagaatgatctgaaagttcatgaggaaaacaccacaattgaagagttagaggcgcttggagagtccttagggaaaggcgatgatcataaggacatggacatcatcaccaaattcctgaagtttcttcttggcgtttgggctaaagaattgaatgccagagaagattatgtgaaacgcagtgtgcagggtaaactgaacagtgcgacccagaaacagaccgagtcctacctaagaccactttttagaaagctacggaaaaggaatcttcctgctgatattaaagaatcaataacggatattattaaattcatgttgcagagagaatacgtgaaggcaaatgatgcttatcttcagatggccattggaaatgcgccttggcccatcggtgtcactatggttggtatccatgccagaactggcagagaaaagattttttccaagcatgttgcacatgttttaaatgacgaaactcagcggaaatatattcagggattgaagaggttaatgaccatttgccagaaacactttcctacagacccatccaaatgtgtggagtacaatgcactgtgagatctgtgtatggtgtgttaataacaataagaaacttagggaagcaggctgtggacttctggaattaccaacaggaatgaggaaagaagaaaactggagtttccagtctctgagttctacctgatgtaactcttgattggttttaagaactttgttggccttcatttcaaaaaaaaaaaaaaaaaaaaaaaaaaaaaaaaa

SEQ ID NO: 16 - protein, 342 aa, Homo sapiens
MDILKSEILRKRQLVEDRNLLVENKKYFKRSELAKKEEEAYFERCGYKIQPKEEDQKPLTSSNPVLELELAEEKLPMTLSRQEVIRRLRERGEPIRLFGETDYDAFQRLRKIEILTPEVNKGLRNDLKAALDKIDQQYLNEIVGGQEPGEEDTQNDLKVHEENTTIEELEALGESLGKGDDHKDMDIITKFLKFLLGVWAKELNAREDYVKRSVQGKLNSATQKQTESYLRPLFRKLRKRNLPADIKESITDIIKFMLQREYVKANDAYLQMAIGNAPWPIGVTMVGIHARTGREKIFSKHVAHVLNDETQRKYIQGLKRLMTICQKHFPTDPSKCVEYNAL

SEQ ID NO: 17 - DNA, 3594 bp, Homo sapiens
cgcgccggcaggggcagcagcgggagcagcgcggggcggagcggcgccagcagccaggagcggcccggcccggcccggcgcggcggcggcggcggcagcggcagcggcagcgacgccagagcccgtgggcgccgttcgcgaggccgccgcagaggcccggccgcagcgcagggaagcctgggggccagaggtcgccgctgccgccatgccgctgctcttcctcgagcgcttcccctggcccagcctccgcacctacacgggcctcagcggcctggccctgctgggcaccatcatcagcgcctaccgcgcgctcagccagcccgaggccggccccggcgagccggaccagctaacggcctcgctgcagcctgagccgccggcgcccgcccggccgagcgccgggggaccccgggcccgcgatgtggcccagtacctgctctcagacagcctcttcgtgtgggttctagtaaataccgcttgctgtgttttgatgttggtggctaagctcatccagtgtattgtgtttggccctcttcgagtgagtgagagacagcatctcaaagacaaattttggaattttattttctacaagttcattttcatctttggtgtgctgaatgtccagacagtggaagaggtggtcatgtggtgcctctggtttgccggacttgtctttctgcacctgatggttcagctctgcaaggatcgatttgaatatctttccttctcgcccaccacgccgatgagcagccacggtcgagtcctgtccctgttggttgccatgctgctttcctgctgtggactggcggccgtctgctccatcaccggctacacccacggaatgcacaccttggctttcatggctgcagagtctcttcttgtgacagtgaggactgctcatgtgattttacgatacgtaattcacctctgggacctcaaccacgaagggacgtgggaaggaaaggggacgtatgtctattacacagactttgtcatggagctcactctcctgtccctggacctcatgcaccatattcacatgttgttatttggcaacatctggttatccatggccagcctggtcatctttatgcagctgcgttacctgtttcatgaggtgcaacgtcgaattcgtcggcacaagaactatctacgtgtggttggaaacatggaggccaggtttgcagttgcaactccagaggagctggctgtcaacaatgacgactgtgccatctgttgggactccatgcaggctgcgcggaaactgccctgtggacatcttttccacaactcctgtcttcgttcctggctagaacaagacacctcctgtccaacatgcagaatgtctcttaatattgccgacaataatcgtgtcagggaagaacatcaaggagagaacttggatgagaatttggttcctgtagcagcagccgaagggagacctcgcttaaaccaacacaatcacttcttccatttcgatgggtctcggattgcgagctggctgccgagtttttcggttgaagtgatgcacaccaccaacattcttggcattacgcaggccagcaactcccagctcaatgcaatggctcatcagattcaagagatgtttccccaggttccataccatctggtactgcaggacctccagctgacacgctcagttgaaataacaacagacaatattttagaaggacggattcaagtaccttttcctacacagcggtcagatagcatcagacctgcattgaacagtcctgtggaaaggccaagcagtgaccaggaagagggagaaacttctgctcagaccgagcgtgtgccactggacctcagtcctcgcctggaggagacgctggacttcggcgaggtggaagtggagcccagtgaggtggaagacttcgaggctcgtgggagccgcttctccaagtctgctgatgagagacagcgcatgctggtgcagcgtaaggacgaactcctccagcaagctcgcaaacgtttcttgaacaaaagttctgaagatgatgcggcctcagagagcttcctcccctcggaaggtgcgtcctctgaccccgtgaccctgcgtcgaaggatgctggctgccgccgcggaacggaggcttcagaagcagcagacctcctagcgctcccttgccttcctcagctgcctcctgcgccctgtgcccgactgactggaggaggcctgtcccaattctgcccgctccatggaaaagcgggcttgactgcattgccgctgtataaagcatgtggtcttatagtgtttggacagctgataaatttaatccttctttgtaatactttctatgtgacatttctcttccccttagaaacactgcaaattttaactgtaggtatgatctcttctggtgttgactggactgcttggggtgggggacgatcaggaggaagtgagcagtcgcctgcctgcagcaggcagcttctactcctgcctcatgcgtacgtcccacaaatgcaggtgtcctgagcaccacacccagtgggaagagtgtgggggaggcgcacagtgtgagcccgcccccacgtcgtggggtaacatctgttatcaaactgctgtcgttgttgtggaagcatgtagactgtgccagaggccagacccacgggctcatgcacccctgagccagcagggcatcttggaaaaggaactcttggttcgatacctggagcagaggaggggaaagtccagggctatagggtgtgatgaagtcacccctttctgtcccactacatctgggactgactttccgagcctccagtccaaagccggcttgatttccgtgaactctggtgctcctgcatctcatgagtgtgccccatgggtcccctcccctctcagcatttccttgtcccgtctggacctggggagtggttaggcagcaagctttggtttatggttttcattcattggtgaagtaaattaggcagtgctaaagcctgtgggtttggtccttgaacaagatgtgggccttgcaagatgggagagtaaaccttgaagggctttattaaagaaataaaaaagaacttttgtatcttttatcctgggagcactgcgttttcctagctgtgttattcctggtttaattcagcagagaaggtaaggtgtgaacctacctgccttggagagggcccaggtcccaaatctcttcaaattcttcacatgtttaactttaaggatttgaaccatgaagtcataggttacagacctcagttttatgccccattggattactttttttttttttttttttttttttttttttactctttgaaagctttgttttgtggtagtccttttgggaagaatccagtattatccacaattattggcaaagtttaaatgtattttacataacggaaagtttttagaatgttgaaaagtaattgaaaaaggtgataggtaaatttttaggcaaagataatttatttcaataaatctttcaaaagccttaccttgaaatgctgttagtaaatttctgtgatttttttttttaatttgttttgctgagagcatagctatttgtttttattgtaaaacaataataataataaaaagcaaactcta

SEQ ID NO: 18 - protein, 643 aa, Homo sapiens
MPLLFLERFPWPSLRTYTGLSGLALLGTIISAYRALSQPEAGPGEPDQLTASLQPEPPAPARPSAGGPRARDVAQYLLSDSLFVWVLVNTACCVLMLVAKLIQCIVFGPLRVSERQHLKDKFWNFIFYKFIFIFGVLNVQTVEEVVMWCLWFAGLVFLHLMVQLCKDRFEYLSFSPTTPMSSHGRVLSLLVAMLLSCCGLAAVCSITGYTHGMHTLAFMAAESLLVTVRTAHVILRYVIHLWDLNHEGTWEGKGTYVYYTDFVMELTLLSLDLMHHIHMLLFGNIWLSMASLVIFMQLRYLFHEVQRRIRRHKNYLRVVGNMEARFAVATPEELAVNNDDCAICWDSMQAARKLPCGHLFHNSCLRSWLEQDTSCPTCRMSLNIADNNRVREEHQGENLDENLVPVAAAEGRPRLNQHNHFFHFDGSRIASWLPSFSVEVMHTTNILGITQASNSQLNAMAHQIQEMFPQVPYHLVLQDLQLTRSVEITTDNILEGRIQVPFPTQRSDSIRPALNSPVERPSSDQEEGETSAQTERVPLDLSPRLEETLDFGEVEVEPSEVEDFEARGSRFSKSADERQRMLVQRKDELLQQARKRFLNKSSEDDAASESFLPSEGASSDPVTLRRRMLAAAAERRLQKQQTS

SEQ ID NO: 19 - DNA, 1569 bp, Homo sapiens
ccgcgggtcacggcagtggcggtgatggcggcggagcccgggcacatggacatgggcgccgaggccctgcccggccccgacgaggccgccgctgccgcagccttcgcagaggtgaccacagtgacagtggccaacgtgggggctgctgcagacaatgtcttcaccacgtctgtggcgaacgcggcatccatctcaggacatgttctgtctggtaggacggcccttcagatcggggacagcctgaacaccgaaaaagcgacactgattgtcgtccacacagatgggagcatcgtggagaccaccgggctgaaaggcccggcagctcccctcaccccaggtcctcagtctcctccaacccctctggctcccggccaagaaaaaggtggaactaaatacaactgggacccttctgtgtacgacagtgacctgcccgtacggtgccggaacatcagcggcactctgtacaagaacaggctcggctcaggcggccggggacggtgcatcaagcagggggagaactggtacagtcccaccgagtttgaggccatggcaggaagagccagcagtaaggactggaaaagaagcattcgctacgcgggccgacccttgcagtgcctcatccaggatgggatcttaaaccctcacgctgcctcttgcacctgtgctgcctgctgcgacgacatgaccttaagtggcccagtcaggctttttgtgccttacaaaaggcgcaagaaggagaatgaactgcccacaactcccgtgaagaaggactcccccaagaacatcacattgcttccagccaccgcggctaccaccttcaccgtgaccccctcgggacagatcacgacctcgggggcactgacctttgaccgagcgtccacggtagaggccactgctgtcatatcatagaatccggcccagggcgacgtcttcgcaggggccacagtccaagaggccagcgtgcagcccccatgcagggccagccaccctgagcctcactaccccggctatcaggacagctgccagatcgcaccgtttccagaagctgcgttgccaacgtcacatcccaaaatagtgttgacatccctgcctgcgctggcggtcccacccccgactcccaccaaagcggcacctcccgcgtaggtcaatgggctggagctgtcagagccgcggagctggctgtacctagaagagatggtcaactccttgctcaacacagcgcagcagctgaagacgctgtttgagcaagccaagcatgccagcacctaccgagaagctgccacaaaccaggccaagatccacgctgacgcagagcggaaggagcagtcctgcgttaactgcggccgggaggctatgagcgagtgcaccggttgccacaaggtcaactactgctccaacttctgccaacgcaaggactggaaggatcaccagcacatatgcggccagtcagcagctgtcaccgtccaggcagacgaagtccacgtggctgaaagcgtgatggagaaggtgaccgtgtgaggctccatcggccgccctgggagcttggggcccctcgcactcctgtgaggc

SEQ ID NO: 20 - protein, 294 aa, Homo sapiens [sequence omitted]

SEQ ID NO: 21 - DNA, 3475 bp, Homo sapiens
gaggaggtcggctttgttctcgccccgccctctccccgcctcctccctagatatcgcgagagggcgggtccgcttggcttcggcgtcgcgtcgctcctgcgctggagctctaggccggcgtctctccgcgagccgcgggtcaagtggcagtggctaagggtgcgcgagtagccgccacccgttcggcgttggctctggcgtcggggtcgttgtgtcgtgacaaccgctgcggtagccgtttccgaggcagcagttgcggccgctttagccctgagcgggatctgcggctgcctgcgagtctctgctgtgccgacccttctcttcgcggaccccacgccaagcagcgaccctgagccgacagccggagcgcccggcaatggcggcctcgacggcctcgcaccggcccatcaaggggatcttgaagaacaagacctctacgacttcctctatggtggcgtcggccgaacagccccgcgggaatgtcgacgaggagctgagcaaaaaatcccagaagtgggatgaaatgaacatcttggcgacgtatcatccagcagacaaagactatggtttaatgaaaatagatgaaccaagcactccttaccatagtatgatgggggatgatgaagatgcctgtagtgacaccgaggccactgaagccatggcgccagacatcttagccaggaaattagctgcagctgaaggcttggagccaaagtatcggattcaggaacaagaaagcagtggagaggaggatagtgacctctcacctgaagaacgagaaaaaaagcgacaatttgaaatgaaaaggaagcttcactacaatgaaggactcaatatcaaactagccagacaattaatttcaaaagacctacatgatgatgatgaagatgaagaaatgttagagactgcagatggagaaagcatgaatacggaagaatcaaatcaaggatctactccaagtgaccaacagcaaaacaaattacgaagttcatagacgagatttgttcaacactgcaattgtttgttagatgtaaaccctgtgactatagtacgttgcttcttgttcttcacaattcatgacttaagtaccaaaatgcataccagttattatatattgccaagaattaaatgataaacttagagactgattagactgaaaatgcctaatcgatatatatattcttgtgcctagtactttaccacaaatacagtgtaatatcatcagtccaaaactgcattacttttgtaaaaacactggttaatttgtataagatattatagagctttttatgctttagaagttaaacaatatctttgggggggaactaatttattttcatcacttgaaatgtggtagctcttacaaagtttattgatttgatttttttaaaaatcaaaagccaattgaacaacaggatatatagactgataaatatttaggctgaatagtattttaacacttgtcttcaacttgatttgtctgtttaattgaaaagaattataagagttactgttgcattttctgacctactatttttaaaattcctgttgagtttctttgtgtttacaaggaaaggactgaactttttctcatcaaaactagcttttttccccacaaataaattatcaggttaaactttcaccaatgtctgctctgttttttgtttttggggggttttttgtttgtttttgttttttaatgtttttggtacactgggcagacttcagagcagttttttaaaaaaataaatattctaatgtagctatctcgccattccctttaaatacctgtcttaacctcctgcttttctttcctactcctttccacacatacgcacacaatcttctaccttttaaaggatcattaagactgtcaccacattaggagctctttctctcactcttctgtcatttgctgctatgttgaaattcttattttgaccatcaatgcctatgaattcttctaatacgtgaagaaaatagtttgagtagcagcagtgctataggtgggaaatacagtttagctgctgaatttctatacctctctgatttacagattgctaattaaatggctattattagtttggattaattagacttaagaaaacaactaactgagggttttttttgtttgttttttgagggttttctttgcatgagagttgtatgtaaccagtgatatgattattcctgaatgtacagacagaagtaagcctggacattgttaaatagtccctgctttaagggactacgataatgtgtactatgacaaacgtgctttattcttctaacgcagtaagaattaggtggaattttttccttcaaccaagtgcaggaaagccctgtgtgtcttggtttagttatggtttcatttctagccatacaattgatgaattgtgtacaacttttgttagtaccaaaataatctgttatatgaacagacttctaaaataacgtctgtatattttatatatagatacatatatgaaagaaggcttttattgaacagcttatcttccacttgcaggtttatggaaacagcagtatttgaaaataaataaaaagtgggagaattccttgctgttagaagaatgtggccattattttgattttttgaatgagatatataatcaaagtactgctgaactgtgagtgcagtattctaaacatttcagctaggaataccactgatttagaaacaaaactgtttgtctctggtttctgaatttaaaatgttgggattacctgtttaaatctgtcttgggggatgtagagattaagtctgtacatgtgcgtgcacatatattcatgcaccctctgattttggttttcttgtttctgagttcttagaaagtacccacatactcttttttttttttttgtagaagtagctgtcatagagtgaagaaaaggataagactttaaacagttgattctttttgtgttttctacaaactattttttgaaatttaaatcacaaactcattttctggtttttagaaagtagatgatgatttcagaggagtaaggcatgccaagcagcatgctcagtggggttttaggctgtcacatgcagctgagaaaaggtatgttcaagtcataagtaggtaattgatagggtatgaactagtcaaaatatgaaccattatgattcaagttagattttcctctggagagacagatctgaatgttcagttctagccaaggtagattttactttcaactttttaatcagtatcactttctgtgcttaactatttggtgttaccttgtctgttttcatttgtctaaaattctgcagagatgactaaaatttgacataatggtgtaaatggattgttaaggtaactttcagttgaagattaaagcaagatgccattttccccatgactttggttttgtttacatttttccctttcagttagtatacactacacgtactgtaataaaatacaataatatgacaaaaaaaaaa

SEQ ID NO: 22 - protein, 205 aa, Homo sapiens
MAASTASHRPIKGILKNKTSTTSSMVASAEQPRGNVDEELSKKSQKWDEMNILATYHPADKDYGLMKIDEPSTPYHSMMGDDEDACSDTEATEAMAPDILARKLAAAEGLEPKYRIQEQESSGEEDSDLSPEEREKKRQFEMKRKLHYNEGLNIKLARQLISKDLHDDDEDEEMLETADGESMNTEESNQGSTPSDQQQNKLRSS

SEQ ID NO: 23 - DNA, 1851 bp, Homo sapiens
tgcagctgttttcactccgctgtgactcagagcgctccgggctgcaggagaggaagaaatcttgcatcaccatggtgcacttctgtggcctactcaccctccaccgggagccagtgccgctgaagagtatctctgtgagcgtgaacatttacgagtttgtggctggtgtgtctgcaactttgaactacgagaatgaggagaaagttcctttggaggccttctttgtgttccccatggatgaagactctgctgtttacagctttgaggccttggtggatgggaagaaaattgtagcagaattacaagacaagatgaaggcccgcaccaactatgagaaagccatctcccagggccaccaggccttcttattggagggggacagcagctccagggatgtcttctcttgcaatgtgggtaacctccaacctgggtcgaaggcggcagtcaccctgaagtatgtgcaggagctgcctctggaagcagatggggctctgcgctttgtgctcccagctgtcctgaatcctagataccagttctctgggtcgtctaaggacagttgccttaatgtgaagactcctatagtccctgtggaggacctgccctacacactcagcatggtcgccaccatagattcccagcatggcattgagaaggtccaatccaactgccccttgagtcctaccgagtacctaggagaggacaagacttctgctcaggtttccctggctgctggacacaagtttgatcgggacgtggaactcctgatttactacaatgaggtgcatacccccagcgtggttttggagatggggatgcctaacatgaagccaggtcatttgatgggagatccatctgcaatggtgagtttctatccaaatatcccagaagatcaaccatcaaatacctgtggagagtttatctttctcatggaccgctcgggaagtatgcagagccccatgagtagccaggatacatctcagctgcgaatacaggcagccaaggaaacactgattttgctgctgaagagtttacctataggctgttatttcaacatctatggatttggctcttcctatgaggcatgctttccggagagtgtgaagtacactcagcaaacaatggaggaggctctggggagagtgaagcttatgcaggccgacctagggggcactgaaatcttggcaccactccagaacatttacaggggaccctccatcccaggccaccccctacagctttttgtctttacagatggagaagttacagacacgtttagtgtaattaaagaagttaggatcaacagacagaaacacaggtaggaagaaaatgtgatttccgggtgattggtgctgagtagtgacacacagactctagtgctacatgatgccggtgttgaccttccttcaagaggaccaaatgatttcagaatttagttttagcagctgaaaatttatttctccctgtaaacgttaaaaacagttttccaaataacatcaacaacacagcaaaaccattgtttcttattctttctaaactacaacgaacacaagaattgaatagtaagatgttaattttttttactataaacatttttagagaagtaaaacatgctgaaaactacacaaattataagcatacaactggactcattatcacagtgaatgcactgtgtgatcgccacataggtaaaaactggaatggtcgtaggcctctccatctgtacccttttccatcatgtcctattccctgtcactacacactaaaactttcctgacttacaataccatgggttatttatgcttgtttttgaatgaaaataaataagttatacagtaaaaaaaaaaaaaaaaa

SEQ ID NO: 24 - protein, 415 aa, Homo sapiens
MVHFCGLLTLHREPVPLKSISVSVNIYEFVAGVSATLNYENEEKVPLEAFFVFPMDEDSAVYSFEALVDGKKIVAELQDKMKARTNYEKAISQGHQAFLLEGDSSSRDVFSCNVGNLQPGSKAAVTLKYVQELPLEADGALRFVLPAVLNPRYQFSGSSKDSCLNVKTPIVPVEDLPYTLSMVATIDSQHGIEKVQSNCPLSPTEYLGEDKTSAQVSLAAGHKFDRDVELLIYYNEVHTPSVVLEMGMPNMKPGHLMGDPSAMVSFYPNIPEDQPSNTCGEFIFLMDRSGSMQSPMSSQDTSQLRIQAAKETLILLLKSLPIGCYFNIYGFGSSYEACFPESVKYTQQTMEEALGRVKLMQADLGGTEILAPLQNIYRGPSIPGHPLQLFVFTDGEVTDTFSVIKEVRINRQKHR

SEQ ID NO: 25 - DNA, 4369 bp, Homo sapiens
gtcccaggcggttccgctcaacagacgctgctgtggctgcgccgggctgcgacactgcagttgtctacgcggccggggccgggacgaggaggcgttggacggggtcgcatacgttcgtcccctcgcattgcggccccgacagctgcgccaggatccccgggcggcggcgcggggcgtgaacgctctggggctcagccaggcctgcgcgggcccgaggccggaggaacccggactccggcgtagcggttttgacacaagggcgcatatcttcaaagcacctagtacctcctaccattgtcaactgatacagaattcgttgttgggaaggactggggaaacagctgtaacatttgccaccctcagaagctgctggtcctgtgtcacaccaccttagcctcttgatcgaggaagattctcgctgaagtctgttaattctactttttgagtacttatgaataaccacgtgtcttcaaaaccatctaccatgaagctaaaacataccatcaaccctattcttttatattttatacattttctaatatcactttatactattttaacatacattccgttttattttttctccgagtcaagacaagaaaaatcaaaccgaattaaagcaaagcctgtaaattcaaaacctgattctgcatacagatctgttaatagtttggatggtttggcttcagtattataccctggatgtgatactttagataaagtttttacatatgcaaaaaacaaatttaagaacaaaagactcttgggaacacgtgaagttttaaatgaggaagatgaagtacaaccaaatggaaaaatttttaaaaaggttattcttggacagtataattggctttcctatgaagatgtctttgttcgagcctttaattttggaaatggattacagatgttgggtcagaaaccaaagaccaacatcgccatcttctgtgagaccagggccgagtggatgatagctgcacaggcgtgttttatgtataattttcagcttgttacattatatgccactctaggaggtccagccattgttcatgcattaaatgaaacagaggtgaccaacatcattactagtaaagaactcttacaaacaaagttgaaggatatagtttctttggtcccacgcctgcggcacatcatcactgttgatggaaagccaccgacctggtccgagttccccaagggcatcattgtgcataccatggctgcagtggaggccctgggagccaaggccagcatggaaaaccaacctcatagcaaaccattgccctcagatattgcagtaatcatgtacacaagtggatccacaggacttccaaagggagtcatgatctcacatagtaacattattgctggtataactgggatggcagaaaggattccagaactaggagaggaagatgtctacattggatatttgcctctggcccatgttctagaattaagtgctgagcttgtctgtctttctcacggatgccgcattggttactcttcaccacagactttagcagatcagtcttcaaaaattaaaaaaggaagcaaaggggatacatccatgttgaaaccaacactgatggcagcagttccggaaatcatggatcggatctacaaaaatgtcatgaataaagtcagtgaaatgagtagttttcaacgtaatctgtttattctggcctataattacaaaatggaacagatttcaaaaggacgtaatactccactgtgcgacagctttgttttccggaaagttcgaagcttgctagggggaaatattcgtctcctgttgtgtggtggcgctccactttctgcaaccacgcagcgattcatgaacatctgtttctgctgtcctgttggtcagggatacgggctcactgaatctgctggggctggaacaatttccgaagtgtgggactacaatactggcagagtgggagcaccattagtttgctgtgaaatcaaattaaaaaactgggaggaaggtggatactttaatactgataagccacaccccaggggtgaaattcttattgggggccaaagtgtgacaatggggtactacaaaaatgaagcaaaaacaaaagctgatttctttgaagatgaaaatggacaaaggtggctctgtactggggatattggagagtttgaacccgatggatgcttaaagattattgatcgtaaaaaggaccttgtaaaactacaggcaggggaatatgtttctcttgggaaagtagaggcagctttgaagaatcttccactagtagataacatttgtgcatatgcaaacagttatcattcttatgtcattggatttgttgtgccaaatcaaaaggaactaactgaactagctcgaaagaaaggacttaaagggacttgggaggagctgtgtaacagttgtgaaatggaaaatgaggtacttaaagtgctttccgaagctgctatttcagcaagtctggaaaagtttgaaattccagtaaaaattcgtttgagtcctgaaccgtggacccctgaaactggtctggtgacagatgccttcaagctgaaacgcaaagagcttaaaacacattaccaggcggacattgagcgaatgtatggaagaaaataattattctcttctggcatcagtttgctacagtgagctcagatcaaataggaaaatacttgaaatgcatgtctcaagctgcaaggcaaactccattcctcatattaaactattacttctcatgacgtcaccatttttaactgacaggattagtaaaacattaagacagcaaacttgtgtctgtctcttctttcattttccccgccaccaacttactttaccacctatgactgtacttgtcagtatgagaatttttctgaatcatattggggaagcagtgattttaaaacctcaagtttttaaacatgatttatatgttctgtataatgttcagtttgtaactttttaaaagtttggatgtatagagggataaataggaaatataagaattggttatttgggggcttttttacttactgtatttaaaaatacaagggtattgatatgaaattatgtaaatttcaaatgcttatgaatcaaatcattgttgaacaaaagatttgttgctgtgtaattattgtcttgtatgcatttgagagaaataaatatacccatacttatgttttaagaagttgagatcttgtgaatatatgcctgtcagtgtcttctttatatatttattttttattagaaaaaatgaagtttggttggtgatgcatgaaacaaaatagcaagagagggttatagtttaatagtaagggagataacacagcatgtgtagcaccagttgataattggtctctagtagcttactgtcaaaatgttcaatgaagtcttctgttcatctgttgaaactaggaaaatacccaaacttaaatggaagaattctgaaagagaggatagaatttaaagaacaagagtatataaagttattctttgaatatttcgttgactatatgtacattgagttatctatatttgtaaacaaattagtcatggaaaattattctatctcaaagtctccttttagtctagataatcattatttcattttaaaattagtgtttttcctagtttgcactgatgcgtgtatggatgtgtgtgagtcagtggtagcttatttaaaaagcaccttatcctttctcccataacctttgtacactaaaaaatgaaagaatttagaatgtatttgatgatagcattctcactaagacacatgagaatttaactttataaccgcgtgagttaagatttaattcataggttttgatgtcattgttgaagttatttgtaattcagaaaccttgcttgtgtgatacatagtctcttcatttattactgcttgtctgttgttatatctggattatcaaaagcaatagtgcaccaattaagatgtgctcaaatcaggacttaaatcataggcaccacatttttcatgtcagactagttactttgttgattctcagttactgtaggcatcaaaaggcaaaaatcaaaaaaaaaaaaaacaaaaacaaaaaaaaagatgaacctaggtctgtgtaaagtaaggggagtgttaggagcagccaggactgtgtagtgtgtgtttggttgcatcacaaacatcgtatgtggagacattgcaatacagtgttttttgttttcaacttttcttgtattgtatatttgtattatgttttgaatgcttttctcttttcataattaaatattaatgtttgggataactgccaagaagaagtaaaaatattgaatggaacttctatatgaggatgctgtgatctaaaaattaaatctcagtgggcggagaaaaaaaaa

SEQ ID NO: 26 - protein, 720 aa, Homo sapiens
MNNHVSSKPSTMKLKHTINPILLYFIHFLISLYTILTYIPFYFFSESRQEKSNRIKAKPVNSKPDSAYRSVNSLDGLASVLYPGCDTLDKVFTYAKNKFKNKRLLGTREVLNEEDEVQPNGKIFKKVILGQYNWLSYEDVFVRAFNFGNGLQMLGQKPKTNIAIFCETRAEWMIAAQACFMYNFQLVTLYATLGGPAIVHALNETEVTNIITSKELLQTKLKDIVSLVPRLRHIITVDGKPPTWSEFPKGIIVHTMAAVEALGAKASMENQPHSKPLPSDIAVIMYTSGSTGLPKGVMISHSNIIAGITGMAERIPELGEEDVYIGYLPLAHVLELSAELVCLSHGCRIGYSSPQTLADQSSKIKKGSKGDTSMLKPTLMAAVPEIMDRIYKNVMNKVSEMSSFQRNLFILAYNYKMEQISKGRNTPLCDSFVFRKVRSLLGGNIRLLLCGGAPLSATTQRFMNICFCCPVGQGYGLTESAGAGTISEVWDYNTGRVGAPLVCCEIKLKNWEEGGYFNTDKPHPRGEILIGGQSVTMGYYKNEAKTKADFFEDENGQRWLCTGDIGEFEPDGCLKIIDRKKDLVKLQAGEYVSLGKVEAALKNLPLVDNICAYANSYHSYVIGFVVPNQKELTELARKKGLKGTWEELCNSCEMENEVLKVLSEAAISASLEKFEIPVKIRLSPEPWTPETGLVTDAFKLKRKELKTHYQADIERMYGRK

SEQ ID NO: 27 - DNA, 1202 bp, Homo sapiens
ccggccagcgggcgggctccccagccaggccgctgcacctgtcagtggagggggaggagaggtttggctgccagattcaactggaaaggaaccagtcccagtccagccgcaacctgggagtgggaagctggaggcagcccagacctcctggagccctgcagtcctggggcagagacagaatcaggacacagctcgaggtcagggccagaggctggagctgagggcctagagtgagagggggcaaggcaagggggggagaggaagagaggcaggattagagagaggaggcaggccagaaagaggagagcaggagagacccaaagagaaacagaaggcagatagagagggagtgagaggcaggagctgagacacgagtcctggaggaagaagaccaaaggaagggggcagagacagaaagggaggtgctaggacaaaactcgaaaggtggccctatcagggaagcagaggagaggccgttctagggaagcccagctccggcacttttggccccaactcccgcaggtctgctggctccaggaaaggtggaggagggagggaggagtgggagaatgtgggcgcagggtgggacatgggcatggccaggggcagcctcactcgggttccaggggtgatgggagagggcactcagggcccagagctcagccttgaccctgacccttgctctctccaatccactccggggctcatgaagggaacaagctggaggagcaggaccctagacctctgcagcccataccaggtctcatggaggggaacaagctggaggagcaggactctagccctccacagtccactccagggctcatgaaggggaacaagcgtgaggagcaggggctgggccccgaacctgcggcgccccagcagcccacggcggaggaggaggccctgatcgagttccaccgctcctaccgagagctcttcgagttcttctgcaacaacaccaccatccacggcgccatccgcctggtgtgctcccagcacaaccgcatgaagacggccttctgggcagtgctgtggctctgcacctttggcatgatgtactggcaattcggcctgcttttcggagagtacttcagctaccccgtcagcctcaacatcaacctcaactcggacaagctcgtcttccccgcagtgaccatctgcaccctcaatccctacaggtcggttagtccctctgcccc

SEQ ID NO: 28 - protein, 669 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 29 - DNA, 3992 bp, Homo sapiens
ataaaagctgcccggggaagccaggagagcgaagggcggacgtactcgccacggcacccaggctgcgcgcacgcggtcccggtgtgcagctggagagcgagcggccaccgggagcccccggcacagcccgcgcccgccccgcaggagcccgcgaagatgccccggcgcagcctgcacgcggcggccgtgctcctgctggtgatcttaaaggaacagccttccagcccggccccagtgaacggttccaagtggacttattttggtcctgatggggagaatagctggtccaagaagtacccgtcgtgtgggggcctgctgcagtcccccatagacctgcacagtgacatcctccagtatgacgccagcctcacgcccctcgagttccaaggctacaatctgtctgccaacaagcagtttctcctgaccaacaatggccattcagtgaagctgaacctgccctcggacatgcacatccagggcctccagtctcgctacagtgccacgcagctgcacctgcactgggggaacccgaatgacccgcacggctctgagcacaccgtcagcggacagcacttcgccgccgagctgcacattgtccattataactcagacctttatcctgacgccagcactgccagcaacaagtcagaaggcctcgctgtcctggctgttctcattgagatgggctccttcaatccgtcctatgacaagatcttcagtcaccttcaacatgtaaagtacaaaggccaggaagcattcgtcccgggattcaacattgaagagctgcttccggagaggaccgctgaatattaccgctaccgggggtccctgaccacacccccttgcaaccccactgtgctctggacagttttccgaaaccccgtgcaaatttcccaggagcagctgctggctttggagacagccctgtactgcacacacatggacgacccttcccccagagaaatgatcaacaacttccggcaggtccagaagttcgatgagaggctggtatacacctccttctcccaagtgcaagtctgtactgcggcaggactgagtctgggcatcatcctctcactggccctggctggcattcttggcatctgtattgtggtggtggtgtccatttggcttttcagaaggaagagtatcaaaaaaggtgataacaagggagtcatttacaagccagccaccaagatggagactgaggcccacgcttgaggtccccggagctcccgggcacatccaggaaggaccttgctttggaccctacacacttcggctctctggacacttgcgacacctcaaggtgttctctgtagctcaatctgcaaacatgccaggcctcagggatcctctgctgggtgcctccttgccttgggaccatggccaccccagagccatccgatcgatggatgggatgcactctcagaccaagcagcaggaattcaaagctgcttgctgtaactgtgtgagattgtgaagtggtctgaattctggaatcacaaaccaagccatgctggtgggccattaatggttggaaaacactttcatccggggctttgccagagcgtgctttcaagtgtcctggaaattctgctgcttctccaagctttcagacaagaatgtgcactctctgcttaggttttgcttgggaaactcaacttctttcctctggagacggggcatctccctctgatttccttctgctatgacaaaacctttaatctgcaccttacaactcggggacaaatggggacaggaaggatcaagttgtagagagaaaaagaaaacaagagatatacattgtgatatattagggacactttcacagtcctgtcctctggatcacagacactgcacagaccttagggaatggcaggttcaagttccacttcttggtggggatgagaagggagagagagctagagggacaaagagaatgagaagacatggatgatctgggagagtctcactttggaatcagaattggaatcacattctgtttatcaagccataatgtaaggacagaataatacaatattaagtccaaatccaacctcctgtcagtggagcagttatgttttatactctacagattttacaaataatgaggctgttccttgaaaatgtgttgttgctgtgtcctggaggagacatgagttccgagatgacccaatctgcctttgaatctggaggaaataggcagaaacaaaatgactgtagaacttattctctgtaggccaaatttcatttcagccacttctgcaggatccctactgccaacctggaatggagacttttatctacttctctctctctgaagatgtcaaatcgtggtttagatcaaatatatttcaagctataaaagcaggaggttatctgtgcagggggctggcatcatgtatttaggggcaagtaataatggaatgctactaagatactccatattcttccccgaatcacacagacagtttctgacaggcgcaactcctccattttcctcccgcaggtgagaaccctgtggagatgagtcagtgccatgactgagaaggaaccgacccctagttgagagcaccttgcagttccccgagaactttctgattcacagtctcattttgacagcatgaaatgtcctcttgaagcatagctttttaaatatctttttccttctactcctccctctgactctaagaattctctcttctggaatcgcttgaacccaggaggcggaggttgcagtaagccaaggtcatgccactgcactctagcctgggtgacagagcgagactccatctcaaaaaaaaaaaaaaaaaaattattctgtaccatcacaacttttcacaacgatggcaagccttatgtcttgggagcctgttttgctaggcaaagttacaagtgacctaatgggagctcaaatgtgtgtgtgtctctctgtgtgtttgtgtgtgtgtgtgcactcaagacctctaacagcctcgaagcctggggtggcatcccggccttgccattagcatgcctcatgcatcatcagatgacaaggacaaccctcatgacgaagcaacatgaattagggggcctcttggccttggtccaaaattgtcaatcagaaatgaacataaaggactccagagcagtgggactgtctgtcaaaagactctgtatatcttttgtggatgagttttgtgagagaacagagagaccattgtacctggcacaagggctgttcatgaaaagggagacttactgggaggtgcaagacagtggcatttctcctctcctcttgctgctcagcacagccctggattgcagccccgaggctgagaccagacaaagcccgggaggcagaaagatgctccaagaaccaacactatcaatgtctttgcaaatcctcacaggattcctgtgggtccagctttggaactgggaaacctttcttcggatccgcactcattccactgatgccagctgcccctgaaggatgccagtactgtggtgtgtgagtctcagcagccgcccacacgctcctaactctgctgcatggcagatgcctaggtggaaatagcaaaaacaaggcccaggctggggccagggccagaggggaaggccctggattctcactcatgtgagatcttgaatctctttctttgttctgtttgtttagttagtatcatctggtaaaatagttaaaaaacaacaaaaaactctgtatctgtttctagcatgtgctgcattgactctattaatcacatttcaaattcaccctacattcctctcctcttcactagcctctctgaaggtgtcctggccagccctggagaagcactggtgtctgcagcacccctcagttcctgtgcctcagcccacaggccactgtgataatggtctgtttagcacttctgtatttattgtaagaatgattataatgaagatacacactgtaactacaagaaattataaatgtttttcacatcaaaaaaaaaaaaaaaaaa

SEQ ID NO: 30 - protein, 354 aa, Homo sapiens
MPRRSLHAAAVLLLVILKEQPSSPAPVNGSKWTYFGPDGENSWSKKYPSCGGLLQSPIDLHSDILQYDASLTPLEFQGYNLSANKQFLLTNNGHSVKLNLPSDMHIQGLQSRYSATQLHLHWGNPNDPHGSEHTVSGQHFAAELHIVHYNSDLYPDASTASNKSEGLAVLAVLIEMGSFNPSYDKIFSHLQHVKYKGQEAFVPGFNIEELLPERTAEYYRYRGSLTTPPCNPTVLWTVFRNPVQISQEQLLALETALYCTHMDDPSPREMINNFRQVQKFDERLVYTSFSQVQVCTAAGLSLGIILSLALAGILGICIVVVVSIWLFRRKSIKKGDNKGVIYKPATKMETEAHA

SEQ ID NO: 31 - DNA, 6054 bp, Homo sapiens
cacgcggctaccgagctggaggaggcggcgggcgcgagacccggaatgcgcagggcccccgcctcgccccccccagcccgggccgcggcccccgccttccccgcagtcgtcccgcactcggtgcccgccccccgaggccggcggctgctcccactcggggccgttgctgcttgtgccgtgagcgccgcccagccattgtccccgtcgctccgtcagccgcgccggaccgcgcaccaggaggcgagagcgcgcatggggagcctctgttgatgccgccgccgcgccgccctccgaggctgcgtcccgggaagcccggctccccgagcgctccggcctggcccggtgccccggacctgagtgcgtccccatggaggcgcccgggctggcccaggcggccgcggcggagagcgactcccgcaaggtggcggaggagacccccgacggggcgcccgcgctctgccccagccctgaggcgctgtcgccggagccgcctgtgtacagcctgcaggactttgacacgctggccaccgtgggcactgggacgttcgggcgggtgcacctggtgaaggagaagacagccaagcatttcttcgccctcaaggtgatgagcattcccgacgtcatccgcctaaagcaggagcaacacgtacacaatgagaagtctgtcctgaaggaagtcagccacccgttcctcatcaggctgttctggacgtggcatgacgagcgcttcctctacatgctcatggagtacgtgccgggcggcgagctcttcagctacctgcgcaaccgggggcgcttctccagcaccacggggctcttctactctgcagagatcatctgtgccatcgagtacctgcactccaaagagatcgtctacagggacttgaagccagagaacatcctgctggatagggatggccacattaagctcacggactttgggttcgccaagaagctggtagacaggacttggaccctctgtggaacacccgagtacctagcccccgaagtcattcagagcaagggccacggaagggccgtggactggtgggccctcggcatcctgatattcgagatgctttcggggtttcctccgttttttgatgacaacccgtttggcatttatcagaaaattcttgcaggcaaaatagatttccccagacatttggatttccatgtaaaagacctcattaagaaactgctcgtggttgacagaacaaggcgattaggaaacatgaagaacggggcgaatgatgtgaagcatcatcggtggttccgctccgtggactgggaagctgttccgcagagaaaactgaagcctcccatcgtgcccaagatagctggtgacggcgacacttccaacttcgaaacttaccctgagaatgactgggacacagccgcgcccgtgccgcagaaggatttagaaatcttcaagaatttctgaggacaggagctcacatctggaagaaacaggaagattggaatctgcctggaacaaagaactgcacctaagcagaccagaagcaaaatgtcttcttcacggcataaggacatctccacttttctctgtacctgtgtgtatagaaatagattagagcacagttgaaattcatggaactggcattatttaagcaactggaattccacactgtaggaaggttttgaaaattgtttggttgtagattttatcttatcctttagtgttgtgttcctactgtgatgtcttggtttttgtcatagacttaagtttataagtttgaactggacttgttcgattataaccacaaattgtgtgtgtgtgtgtgtgtgtgtgtgtgtgtgtgtgtgtgtgtgtatgcctgtgtgtatatatagaagtcattatggcagatgcacagaaattgtgcagtgatgtaaatgttcatactttacagagcctataatttttatttttcaatttgttttttcaaaaatctcttctcggggacaacatctgaagggtatgttgcatgcattaaaaaaaatcatctcacatgcattttatagttttggggaagaaaatatcatggggaggtctaccttcagtatctttagtgcttcttacctggtaacttgagactttaaaagaagaaacaaagaggggaagatatgggagcgaatttattccaagaatctacaatgacattgaagttgttggaggaatgtactgtatttaaaaaaaccttctgtgacacattcaaaaatttcatctgagctggatgcagtggcttgttcctatagtcccagcactttgggaggctgaggtgggtggattgcttgagcccaggagttggagaccagtctgggaaacgtggtgagacctcatctctacaaaatacaaaaaaattagccgggcatggtggcacgtgagtgtagtctcagctactcaggaggctgagatgagaggatcacttgagcctggggaggtccaggccgcagtgatccgagatcacaccactgcattccagcctgggtgacagagtgagaccctgtccaaaaaaaaaaaaaaaaaaaaagaaggcaatggctttgtgcttttgaagatggattgaagagaacgtccaccttaaggctttaaaagacagtgaagctgggtgcggtggtgcactcctgtaaccctgggactttgggaagctgaggcaggaagattgagcctaggagttcgagaccgacctgggcagcatagcgagaccccatctctatataaaaaaaaagatgttaaaacagtgaaattacagaaacagaacatagccaccctttttcatcagtgaccttttcttcacgtggaggtcaactcagtaggtgagaataattagtaggttcagcagaataaactctttacaattaataactcagtggaaaatgatgttcagatggtgaaatgtggaaatgttttagacaactctattttcagcctgtgcttctcactcaccatcctgttgggattaacagttgagggccatcgctgcctaaacatttagcgtgcggtttcccatcagttttaccgtgaatgtgaaaaggtgaaactggtgctgacttcggcagcaggtatactaaaattggaatgacacagaggagattagcatggccccctgcgcgaggaagatctgcagattcatgcagcattccatatttttttaagaaaaggttacactgtgcaccgacatgtggagctagaaggaaaagcttttaagtctcatgtttgctgactataaaaataaatcatagtaataatattgaacattctactttaaaaattattaataaaaccataaatgaaacacttgagcacactttaaaaagtacataaatccatgagtccatatgcctatttttaggttgtctgttttattatccaggatttcatactcagatgagaattagcatcaggaaagcctgaaatgccttaattgtgaactgatatgcaggaaaatgaaatatgcaagcacattggtgaccttgttctttacagacgtcgtgatcagcctccagccatacagaacaaatgagaaattagaagcgattccaagaacgtgtgatggtgtgaaacccactttggaccatcacacacatttcagcatttgaagtgacacaagctacctggtaactgcccatggcggggtcatagtttaaatggcccaatgtcatgtagaggttaatttaatcaaaatgagctataaacatggagcaatcactgtataattttaaaatagtttacctggccaggcatggtggctcacgcctgtaatcccagcactttgggaggccgaggcaggtggatcacctgaggtcaggagttcgagaccagcctggccaacatggcaaaacccccatctctactaaaatacaaaaattaggtgtggtggcgggcacctgtaatcctggctactcgggaggctgaggcaggagaatcgcttgaacccgggaggcagaggctgcagtaagccgagatcgtgccactgcactccagcctgggccacagagcaagactccgtctcaaaacataaaaataaaaaaaatacaaatagctgacccgggtgggcattccaaaatgtccgcatggacgatcacgaaacaatcacggtgcgaaatgtccgcatggatgatcacgaaacaatcacggtgcagaatgtccgtatggatgatcatgacaaacaatcactgcgcaaaatgtggaggaaaattcattaccataatcacaacatttgtatcagaaaaacattacaaatttactttgcttcatgtaattttacgagttggcaatattttcttaaattggaatactctaagagaataggacatggatatgttacaagaagaagtgtattggggcaagtcttgtttttctgaagggaaatgccaccggcctgctttcataggcattgataaagccaggaaggaaataaagtcacaaccaaatagtatacactgtctctactgaattcttctaattccttcgcaacagaagtctaaatcctgcttagcctaaacatatctatagccagcgatgggcaaattatatttgctgtcagcatgatagaataagaagcagaaaccccccagcagtggggtaagattgcgcgcttcatcccctcaaaatgcagttcagccccactccctctccatgcccatcaaacaactgcacaatcacttgttactgaatttcccattcagcgttttgtttctttctgtccatacttaaagaccaaatcatttgtttgctttgaagaaaaagcaaaaacaagaacaaaaacaccaaatcatttgaaatcactgtccattaaaaaaaatcaccagtggaactaaacagttgagtggtttgggatgtgttcgccatttctgtgtttgacctacatggcctctaattctttggaccattgtaactgtgaaactatggcttaatactgttaaagaccccaaggctccactctgtagccaggtgcacctcaggctgcacaaataccccaccagaatgtcacaatcattcattgctccagctactgagtgaacagggtgcaagacagcactggcttgggttgtatttttgcagccagttaactttgcatcaggtctacacagtccttgcagcctttgatttctccagttgtagagatttgaaatggggtgcccgggttcctctcgtttttccttcaatctacctagcatgcattttacacacattggaggagtcgtatgcactgtttttctgttgctgatggaggcaagcaggccttctaattagtctaacttaaacggatgggattcacttaacaaaagcaagaagaaaagcacacgaacaggtgtcttaatgcatttggatagtttctttgcggggtgtgtgtgtgtgtgtgtgtgtgtgtgtgtgtgtgtgtgtgtgtgtgtcttggtgaaggtgaggggctgtattttcaccaagccttttcataatcaagtttgctttgcaaaccttatggccttgcaccccgcagaacagtcccttcctaatagcagggatgctctgtcatggttttctgtaaattatcgtgtgttgggaagttctgttgaggcttagtttgatcttccatggtggacacgtctgttctgtatgtaaaaggcattacaattgtgttttagcagatgagacttgaagcctttccacagtccttgtgctctgagatggctgttgagctctgctcaactgtcgtagctgaattctttctttgcgctgaacactgggcagcctcaccatgttgccaacgtgcttctggggcccctgtcactgccgccggatgccgcgcacacaggcagagtgccttggcaggtttgtgcaccccttggcgagccagaactgggaaccccgcggggtgaccccttctcatggggggtcggggaggggagtgcctatttttaatcctgtctgtttgttgcacaatggaaatcactgtgatttgtacatatgccctaggaaaattttactgctgtctaatttatgtaataatactgttgattccaggtttgtttaataaaactttgtatcttttcagaaaaaaaaaaaaa

SEQ ID NO: 32 - protein, 358 aa, Homo sapiens
MEAPGLAQAAAAESDSRKVAEETPDGAPALCPSPEALSPEPPVYSLQDFDTLATVGTGTFGRVHLVKEKTAKHFFALKVMSIPDVIRLKQEQHVHNEKSVLKEVSHPFLIRLFWTWHDERFLYMLMEYVPGGELFSYLRNRGRFSSTTGLFYSAEIICAIEYLHSKEIVYRDLKPENILLDRDGHIKLTDFGFAKKLVDRTWTLCGTPEYLAPEVIQSKGHGRAVDWWALGILIFEMLSGFPPFFDDNPFGIYQKILAGKIDFPRHLDFHVKDLIKKLLVVDRTRRLGNMKNGANDVKHHRWFRSVDWEAVPQRKLKPPIVPKIAGDGDTSNFETYPENDWDTAAPVPQKDLEIFKNF

SEQ ID NO: 33 - DNA, 685 bp, Homo sapiens
gccaaaacagtgggggctgaactgacctctcccctttgggagagaaaaactgtctgggagcttgacaaaggcatgcaggagagaacaggagcagccacagccaggagggagagccttccccaagcaaacaatccagagcagctgtgcaaacaacggtgcataaatgaggcctcctggaccatgaagcgagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccacccgaggacagttcttcgtgcctgaaaaaaaaaaaaaaaaaa

SEQ ID NO: 34 - protein, 130 aa, Homo sapiens
MQERTGAATARRESLPQANNPEQLCKQRCINEASWTMKRVLSCVPEPTVVMAARALCMLGLVLALLSSSSAEEYVGLSANQCAVPAKDRVDCGYPHVTPKECNNRGCCFDSRIPGVPWCFKPLQEAECTF

SEQ ID NO: 35 - DNA, 2872 bp, Homo sapiens
tccaggaatcgatagtgcattcgtgcgcgcggccgcccgtcgcttcgcacagggctggatggttgtattgggcagggtggctccaggatgttaggaactgtgaagatggaagggcatgaaaccagcgactggaacagctactacgcagacacgcaggaggcctactcctcggtcccggtcagcaacatgaactcaggcctgggctccatgaactccatgaacacctacatgaccatgaacaccatgactacgagcggcaacatgaccccggcgtccttcaacatgtcctatgccaacccggccttaggggccggcctgagtcccggcgcagtagccggcatgccggggggctcggcgggcgccatgaacagcatgactgcggccggcgtgacggccatgggtacggcgctgagcccgagcggcatgggcgccatgggtgcgcagcaggcggcctccatgatgaatggcctgggcccctacgcggccgccatgaacccgtgcatgagccccatggcgtacgcgccgtccaacctgggccgcagccgcgcgggcggcggcggcgacgccaagacgttcaagcgcagttacccgcacgccaagccgccctactcgtacatctcgctcatcaccatggccatccagcgggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggccccgggcccggccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcctagctcccgggactggggggtttgtctggcatagccatgctggtagcaagagagaaaaaatcaacagcaaacaaaaccacacaaaccaaaccgtcaacagcataataaaatccaacaactatttttatttcatttttcatgcacaaccttgcccccagtgcaaaagactgttactttattattgtattcaaaattcattgtgtatattactacaaagacggccccaaaccaatttttttcctgcgaagtttaatgatccacaagtgtatatatgaaattctcctccttccttgcccccctctctttcttccctcttggccctccagacattctagtttgtggagggttatttaaaaaacaaaaaggaagatggtcaagtttgtaaaatatttgtttgtgcttttcccccctccttacctgaccccctacgagtttacaggcttgtggcaatactcttaaccataagaattgaaatggtgaagaaacaagtatacactagaggctcttaaaagtattgaaaagacaatactgctgttatatagcaagacataaacagattataaacatcagagccatttgcttctcagtttacatttctgatacatgcagatagcagatgtctttaaatgaaatacatgtatattgtgtatggacttaattatgcacatgctcagatgtgtagacatcctccgtatatttacataacatatagaggtaatagataggtgatatacgtgatacgttctcaagagttgcttgaccgaaagttacaaggaccccaacccctttgctctctacccacagatggccctgggaacaatcctcaggaattgccctcaagaactcgcttctttgctttgagagtgccatggtcatgtcattctgaggtacataacacataaattagtttctatgagtgtataccatttaaagattttttcagtaaagggaatattacatgttgggaggaggagataagttatagggagctggatttcaaacggtggtccaagattcaaaaatcctattgatagtggccattttaatcattgccatcgtgtgcttgtttcatccagtgttatgcactttccacagttggtgttagtatagccagagggtttcattattatttctctttgctttctcaatgttaatttattgcatggtttattctttttctttacagctgaaattgctttaaatgatggttaaaattacaaattaaattgggaatttttatcaatgtgattgtaattaaaaatattttgatttaaataacaaaaataataccagattttaagccgcggaaaatgttcttgatcatttgcagttaaggactttaaataaatcaaatgttaacaaaaaa

SEQ ID NO: 36 - protein, 473 aa, Homo sapiens
MLGTVKMEGHETSDWNSYYADTQEAYSSVPVSNMNSGLGSMNSMNTYMTMNTMTTSGNMTPASFNMSYANPALGAGLSPGAVAGMPGGSAGAMNSMTAAGVTAMGTALSPSGMGAMGAQQAASMMNGLGPYAAAMNPCMSPMAYAPSNLGRSRAGGGGDAKTFKRSYPHAKPPYSYISLITMAIQRAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGGGSGSGGSGAKGGPESRKDPSGASNPSADSPLHRGVHGKTGQLEGAPAPGPAASPQTLDHSGATATGGASELKTPASSTAPPISSGPGALASVPASHPAHGLAPHESQLHLKGDPHYSFNHPFSINNLMSSSEQQHKLDFKAYEQALQYSPYGSTLPASLPLGSASVTTRSPIEPSALEPAYYQGVYSRPVLNTS

SEQ ID NO: 37 - DNA, 1923 bp, Homo sapiens
atggcccgaagaccccggcacagcatatatagcagtgacgaggatgatgaggactttgagatgtgtgaccatgactatgatgggctgcttcccaagtctggaaagcgtcacttggggaaaacaaggtggacccgggaagaggatgaaaaactgaagaagctggtggaacagaatggaacagatgactggaaagttattgccaattatctcccgaatcgaacagatgtgcagtgccagcaccgatggcagaaagtactaaaccctgagctcatcaagggtccttggaccaaagaagaagatcagagagtgatagagcttgtacagaaatacggtccgaaacgttggtctgttattgccaagcacttaaaggggagaattggaaaacaatgtagggagaggtggcataaccacttgaatccagaagttaagaaaacctcctggacagaagaggaagacagaattatttaccaggcacacaagagactggggaacagatgggcagaaatcgcaaagctactgcctggacgaactgataatgctatcaagaaccactggaattctacaatgcgtcggaaggtcgaacaggaaggttatctgcaggagtcttcaaaagccagccagccagcagtggccacaagcttccagaagaacagtcatttgatgggttttgctcaggctccgcctacagctcaactccctgccactggccagcccactgttaacaacgactattcctattaccacatttctgaagcacaaaatgtctccagtcatgttccataccctgtagcgttacatgtaaatatagtcaatgtccctcagccagctgccgcagccattcagagacactataatgatgaagaccctgagaaggaaaagcgaataaaggaattagaattgctcctaatgtcaaccgagaatgagctaaaaggacagcaggtgctaccaacacagaaccacacatgcagctaccccgggtggcacagcaccaccattgccgaccacaccagacctcatggagacagtgcacctgtttcctgtttgggagaacaccactccactccatctctgccagcggatcctggctccctacctgaagaaagcgcctcgccagcaaggtgcatgatcgtccaccagggcaccattctggataatgttaagaacctcttagaatttgcagaaacactccaatttatagattctttcttaaacacttccagtaaccatgaaaactcagacttggaaatgccttctttaacttccacccccctcattggtcacaaattgactgttacaacaccatttcatagagaccagactgtgaaaactcaaaaggaaaatactgtttttagaaccccagctatcaaaaggtcaatcttagaaagctctccaagaactcctacaccattcaaacatgcacttgcagctcaagaaattaaatacggtcccctgaagatgctacctcagacaccctctcatctagtagaagatctgcaggatgtgatcaaacaggaatctgatgaatctggaattgttgctgagtttcaagaaaatggaccacccttactgaagaaaatcaaacaagaggtggaatctccaactgataaatcaggaaacttcttctgctcacaccactgggaaggggacagtctgaatacccaactgttcacgcagacctcgcctgtggcagatgcaccgaatattcttacaagctccgttttaatggcaccagcatcagaagatgaagacaatgttctcaaagcatttacagtacctaaaaacaggtccctggcgagccccttgcagccttgtagcagtacctgggaacctgcatcctgtggaaagatggaggagcagatgacatcttccagtcaagctcgtaaatacgtgaatgcattctcagcccggacgctggtcatgtga

SEQ ID NO: 38 - protein, 640 aa, Homo sapiens
MARRPRHSIYSSDEDDEDFEMCDHDYDGLLPKSGKRHLGKTRWTREEDEKLKKLVEQNGTDDWKVIANYLPNRTDVQCQHRWQKVLNPELIKGPWTKEEDQRVIELVQKYGPKRWSVIAKHLKGRIGKQCRERWHNHLNPEVKKTSWTEEEDRIIYQAHKRLGNRWAEIAKLLPGRTDNAIKNHWNSTMRRKVEQEGYLQESSKASQPAVATSFQKNSHLMGFAQAPPTAQLPATGQPTVNNDYSYYHISEAQNVSSHVPYPVALHVNIVNVPQPAAAAIQRHYNDEDPEKEKRIKELELLLMSTENELKGQQVLPTQNHTCSYPGWHSTTIADHTRPHGDSAPVSCLGEHHSTPSLPADPGSLPEESASPARCMIVHQGTILDNVKNLLEFAETLQFIDSFLNTSSNHENSDLEMPSLTSTPLIGHKLTVTTPFHRDQTVKTQKENTVFRTPAIKRSILESSPRTPTPFKHALAAQEIKYGPLKMLPQTPSHLVEDLQDVIKQESDESGIVAEFQENGPPLLKKIKQEVESPTDKSGNFFCSHHWEGDSLNTQLFTQTSPVADAPNILTSSVLMAPASEDEDNVLKAFTVPKNRSLASPLQPCSSTWEPASCGKMEEQMTSSSQARKYVNAFSARTLVM

SEQ ID NO: 39 - DNA, 3310 bp, Homo sapiens
agagccactccatctgagggtggctgcgtgtccacatacgaggggacagggctgaggatgaggagaaccctggggacccagaagaccgtgccttgcctggaagtcctgcctgtaggcctgaaggacttgccctaacagagcctcaacaactacctggtgattcctacttcagccccttggtgtgagcagcttctcaacatgaactacagcctccacttggccttcgtgtgtctgagtctcttcactgagaggatgtgcatccaggggagtcagttcaacgtcgaggtcggcagaagtgacaagctttccctgcctggctttgagaacctcacagcaggatataacaaatttctcaggcccaattttggtggagaacccgtacagatagcgctgactctggacattgcaagtatctctagcatttcagagagtaacatggactacacagccaccatatacctccgacagcgctggatggaccagcggctggtgtttgaaggcaacaagagcttcactctggatgcccgcctcgtggagttcctctgggtgccagatacttacattgtggagtccaagaagtccttcctccatgaagtcactgtgggaaacaggctcatccgcctcttctccaatggcacggtcctgtatgccctcagaatcacgacaactgttgcatgtaacatggatctgtctaaataccccatggacacacagacatgcaagttgcagctggaaagctggggctatgatggaaatgatgtggagttcacctggctgagagggaacgactctgtgcgtggactggaacacctgcggcttgctcagtacaccatagagcggtatttcaccttagtcaccagatcgcagcaggagacaggaaattacactagattggtcttacagtttgagcttcggaggaatgttctgtatttcattttggaaacctacgttccttccactttcctggtggtgttgtcctgggtttcattttggatctctctcgattcagtccctgcaagaacctgcattggagtgacgaccgtgttatcaatgaccacactgatgatcgggtcccgcacttctcttcccaacaccaactgcttcatcaaggccatcgatgtgtacctggggatctgctttagctttgtgtttggggccttgctagaatatgcagttgctcactacagttccttacagcagatggcagccaaagatagggggacaacaaaggaagtagaagaagtcagtattactaatatcatcaacagctccatctccagctttaaacggaagatcagctttgccagcattgaaatttccagcgacaacgttgactacagtgacttgacaatgaaaaccagcgacaagttcaagtttgtcttccgagaaaagatgggcaggattgttgattatttcacaattcaaaaccccagtaatgttgatcactattccaaactactgtttcctttgatttttatgctagccaatgtattttactgggcatactacatgtatttttgagtcaatgttaaatttcttgcatgccataggtcttcaacaggacaagataatgatgtaaatggtattttaggccaagtgtgcacccacatccaatggtgctacaagtgactgaaataatatttgagtctttctgctcaaagaatgaagctccaaccattgttctaagctgtgtagaagtcctagcattataggatcttgtaatagaaacatcagtccattcctctttcatcttaatcaaggacattcccatggagcccaagattacaaatgtactcagggctgtttattcggtggctccctggtttgcatttacctcatataaagaatgggaaggagaccattgggtaaccctcaagtgtcagaagttgtttctaaagtaactatacatgttttttactaaatctctgcagtgcttataaaatacattgttgcctatttagggagtaacattttctagtttttgtttctggttaaaatgaaatatgggcttatgtcaattcattggaagtcaatgcactaactcaataccaagatgagtttttaaataatgaatattatttaataccacaacagaattatccccaatttccaataagtcctatcattgaaaattcaaatataagtgaagaaaaaattagtagatcaacaatctaaacaaatccctcggttctaagatacaatggattccccatactggaaggactctgaggctttattcccccactatgcatatcttatcattttattattatacacacatccatcctaaactatactaaagcccttttcccatgcatggatggaaatggaagatttttttttaacttgttctagaagtcttaatatgggctgttgccatgaaggcttgcagaattgagtccattttctagctgcctttattcacatagtgatggggtactaaaagtactgggttgactcagagagtcgctgtcattctgtcattgctgctactctaacactgagcaacactctcccagtggcagatcccctgtatcattccaagaggagcattcatccctttgctctaatgatcaggaatgatgcttattagaaaacaaactgcttgacccaggaacaagtggcttagcttaagtaaacttggctttgctcagatccctgatccttccagctggtctgctatgagtggcttatcccgcatgagcaggagcgtgctggccctgagtactgaactttctgagtaacaatgagatacgttacagaacctatgttcaggttgcgggtgagctgccctctccaaatccagccagagatgcacattcctcggccagtctcagccaacagtaccaaaagtgatttttgagtgtgccagggtaaaggcttccagttcagcctcagttattttagacaatctcgccatctttaatttcttagcttcctgttctaataaatgcacggctttacctttcctgtcagaaataaaccaaggctctaaaagatgatttcccttctgtaactccctagagccacaggttctcattccttttcccattatacttctcacaattcagtttctatgagtttgatcacctgatttttttaacaaaatatttctaacgggaatgggtgggagtgctggtgaaaagaggtgaaatgtggttgtatgagccaatcatatttgtgattttttaaaaaaagtttaaaaggaaatatctgttctgaaaccccacttaagcattgtttttatataaaaacaatgataaagatgtgaaactgtgaaataaatataccatattagctacccaccaaa

SEQ ID NO: 40 - protein, 440 aa, Homo sapiens
MNYSLHLAFVCLSLFTERMCIQGSQFNVEVGRSDKLSLPGFENLTAGYNKFLRPNFGGEPVQIALTLDIASISSISESNMDYTATIYLRQRWMDQRLVFEGNKSFTLDARLVEFLWVPDTYIVESKKSFLHEVTVGNRLIRLFSNGTVLYALRITTTVACNMDLSKYPMDTQTCKLQLESWGYDGNDVEFTWLRGNDSVRGLEHLRLAQYTIERYFTLVTRSQQETGNYTRLVLQFELRRNVLYFILETYVPSTFLVVLSWVSFWISLDSVPARTCIGVTTVLSMTTLMIGSRTSLPNTNCFIKAIDVYLGICFSFVFGALLEYAVAHYSSLQQMAAKDRGTTKEVEEVSITNIINSSISSFKRKISFASIEISSDNVDYSDLTMKTSDKFKFVFREKMGRIVDYFTIQNPSNVDHYSKLLFPLIFMLANVFYWAYYMYF

SEQ ID NO: 41 - DNA, 6450 bp, Homo sapiens
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 42 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 43 - DNA, 1701 bp, Homo sapiens
ccgcatcctagccgccgactcacacaaggcaggtgggtgaggaaatccagagttgccatggagaaaattccagtgtcagcattcttgctccttgtggccctctcctacactctggccagagataccacagtcaaacctggagccaaaaaggacacaaaggactctcgacccaaactgccccagaccctctccagaggttggggtgaccaactcatctggactcagacatatgaagaagctctatataaatccaagacaagcaacaaacccttgatgattattcatcacttggatgagtgcccacacagtcaagctttaaagaaagtgtttgctgaaaataaagaaatccagaaattggcagagcagtttgtcctcctcaatctggtttatgaaacaactgacaaacacctttctcctgatggccagtatgtccccaggattatgtttgttgacccatctctgacagttagagccgatatcactggaagatattcaaatcgtctctatgcttacgaacctgcagatacagctctgttgcttgacaacatgaagaaagctctcaagttgctgaagactgaattgtaaagaaaaaaaatctccaagcccttctgtctgtcaggccttgagacttgaaaccagaagaagtgtgagaagactggctagtgtggaagcatagtgaacacactgattaggttatggtttaatgttacaacaactattttttaagaaaaacaagttttagaaatttggtttcaagtgtacatgtgtgaaaacaatattgtatactaccatagtgagccatgattttctaaaaaaaaaaataaatgttttgggggtgttctgttttctccaacttggtctttcacagtggttcgtttaccaaataggattaaacacacacaaaatgctcaaggaagggacaagacaaaaccaaaactagttcaaatgatgaagaccaaagaccaagttatcatctcaccacaccacaggttctcactagatgactgtaagtagacacgagcttaatcaacagaagtatcaagccatgtgctttagcataaaagaatatttagaaaaacatcccaagaaaatcacatcactacctagagtcaactctggccaggaactctaaggtacacactttcatttagtaattaaattttagtcagattttgcccaacctaatgctctcagggaaagcctctggcaagtagctttctccttcagaggtctaatttagtagaaaggtcatccaaagaacatctgcactcctgaacacaccctgaagaaatcctgggaattgaccttgtaatcgatttgtctgtcaaggtcctaaagtactggagtgaaataaattcagccaacatgtgactaattggaagaagagcaaagggtggtgacgtgttgatgaggcagatggagatcagaggttactagggtttaggaaacgtgaaaggctgtggcatcagggtaggggagcattctgcctaacagaaattagaattgtgtgttaatgtcttcactctatacttaatctcacattcattaatatatggaattcctctactgcccagcccctcctgatttctttggcccctggactatggtgctgtatataatgctttgcagtatctgttgcttgtcttgattaacttttttggataaaaccttttttgaacagaaaaaaaaaaaaaaaaaaaa

SEQ ID NO: 44 - protein, 175 aa, Homo sapiens
MEKIPVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 45 - DNA, 3070 bp, Homo sapiens
ggcgccgtcttgatactttcagaaagaatgcattccctgtaaaaaaaaaaaaaaaatactgagagagggagagagagagagaagaagagagagagacggagggagagcgagacagagcgagcaacgcaatctgaccgagcaggtcgtacgccgccgcctcctcctcctctctgctcttcgctacccaggtgacccgaggagggactccgcctccgagcggctgaggaccccggtgcagaggagcctggctcgcagaattgcagagtcgtcgcccctttttacaacctggtcccgttttattctgccgtacccagtttttggatttttgtcttccccttcttctctttgctaaacgacccctccaagataatttttaaaaaaccttctcctttgctcacctttgcttcccagccttcccatccccccaccgaaagcaaatcattcaacgacccccgaccctccgacggcaggagccccccgacctcccaggcggaccgccctccctccccgcgcgcgggttccgggcccggcgagagggcgcgagcacagccgaggccatggaggtgacggcggaccagccgcgctgggtgagccaccaccaccccgccgtgctcaacgggcagcacccggacacgcaccacccgggcctcagccactcctacatggacgcggcgcagtacccgctgccggaggaggtggatgtgctttttaacatcgacggtcaaggcaaccacgtcccgccctactacggaaactcggtcagggccacggtgcagaggtaccctccgacccaccacgggagccaggtgtgccgcccgcctctgcttcatggatccctaccctggctggacggcggcaaagccctgggcagccaccacaccgcctccccctggaatctcagccccttctccaagacgtccatccaccacggctccccggggcccctctccgtctaccccccggcctcgtcctcctccttgtcggggggccacgccagcccgcacctcttcaccttcccgcccaccccgccgaaggacgtctccccggacccatcgctgtccaccccaggctcggccggctcggcccggcaggacgagaaagagtgcctcaagtaccaggtgcccctgcccgacagcatgaagctggagtcgtcccactcccgtggcagcatgaccgccctgggtggagcctcctcgtcgacccaccaccccatcaccacctacccgccctacgtgcccgagtacagctccggactcttcccccccagcagcctgctgggcggctcccccaccggcttcggatgcaagtccaggcccaaggcccggtccagcacagaaggcagggagtgtgtgaactgtggggcaacctcgaccccactgtggcggcgagatggcacgggacactacctgtgcaacgcctgcgggctctatcacaaaatgaacggacagaaccggcccctcattaagcccaagcgaaggctgtctgcagccaggagagcagggacgtcctgtgcgaactgtcagaccaccacaaccacactctggaggaggaatgccaatggggaccctgtctgcaatgcctgtgggctctactacaagcttcacaatattaacagacccctgactatgaagaaggaaggcatccagaccagaaaccgaaaaatgtctagcaaatccaaaaagtgcaaaaaagtgcatgactcactggaggacttccccaagaacagctcgtttaacccggccgccctctccagacacatgtcctccctgagccacatctcgcccttcagccactccagccacatgctgaccacgcccacgccgatgcacccgccatccagcctgtcctttggaccacaccacccctccagcatggtcaccgccatgggttagagccctgctcgatgctcacagggcccccagcgagagtccctgcagtccctttcgacttgcatttttgcaggagcagtatcatgaagcctaaacgcgatggatatatgtttttgaaggcagaaagcaaaattatgtttgccactttgcaaaggagctcactgtggtgtctgtgttccaaccactgaatctggaccccatctgtgaataagccattctgactcatatcccctatttaacagggtctctagtgctgtgaaaaaaaaaatgctgaacattgcatataacttatattgtaagaaatactgtacaatgactttattgcatctgggtagctgtaaggcatgaaggatgccaagaagtttaaggaatatgggagaaatagtgtggaaattaagaagaaactaggtctgatattcaaatggacaaactgccagttttgtttcctttcactggccacagttgtttgatgcattaaaagaaaataaaaaaaagaaaaaagagaaaagaaaaaaaaagaaaaaagttgtaggcgaatcatttgttcaaagctgttggcctctgcaaaggaaataccagttctgggcaatcagtgttaccgttcaccagttgccgttgagggtttcagagagcctttttctaggcctacatgctttgtgaacaagtccctgtaattgttgtttgtatgtataattcaaagcaccaaaataagaaaagatgtagatttatttcatcatattatacagaccgaactgttgtataaatttatttactgctagtcttaagaactgctttctttcgtttgtttgtttcaatattttccttctctctcaatttttggttgaataaactagattacattcagttggcctaaggtggttgtgctcggagggtttcttgtttcttttccattttgtttttggatgatatttattaaatagcttctaagagtccggcggcatctgtcttgtccctattcctgcagcctgtgctgagggtagcagtgtatgagctaccagcgtgcatgtcagcgaccctggcccgacaggccacgtcctgcaatcggcccggctgcctcttcgccctgtcgtgttctgtgttagtgatcactgcctttaatacagtctgttggaataatattataagcataataataaagtgaaaatattttaaaactacaa

SEQ ID NO: 46 - protein, 444 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 47 - DNA, 3946 bp, Homo sapiens
cgagaaaaggtgacgcggggcccgggcaggcggccggcgcgcggccccccccccccccgccctggttatttggccgccttcgccggcagctcagggcagagtctcctggaaggcgcaggcagtgtggcgagaagggcgcctgcttgttctttctttttgtctgctttcccccgtttgcgcctggaagctgcgccgcgagttcctgcaaggcggtctgccgcggccgggcccggccttctcccctcgcagcgaccccgcctcgcggccgcgcgggccccgaggtagcccgaggcgccggaggagccagccccagcgagcgccgggagaggcggcagcgcagccggacgcacagcgcagcgggccggcaccagctcggccgggcccggactcggactcggcggccggcgcggcgcggcccggcccgagcgagggtggggggcggcgggcggcgcggggcggcggcgagcgggggccatgcaggcgcgctactccgtgtccagccccaactccctgggagtggtgccctacctcggcggcgagcagagctactaccgcgcggcggccgcggcggccgggggcggctacaccgccatgccggcccccatgagcgtgtactcgcaccctgcgcacgccgagcagtacccgggcggcatggcccgcgcctacgggccctacacgccgcagccgcagcccaaggacatggtgaagccgccctatagctacatcgcgctcatcaccatggccatccagaacgccccggacaagaagatcaccctgaacggcatctaccagttcatcatggaccgcttccccttctaccgggacaacaagcagggctggcagaacagcatccgccacaacctctcgctcaacgagtgcttcgtcaaggtgccgcgcgacgacaagaagccgggcaagggcagctactggacgctggacccggactcctacaacatgttcgagaacggcagcttcctgcggcggcggcggcgcttcaagaagaaggacgcggtgaaggacaaggaggagaaggacaggctgcacctcaaggagccgcccccgcccggccgccagcccccgcccgcgccgccggagcaggccgacggcaacgcgcccggtccgcagccgccgcccgtgcgcatccaggacatcaagaccgagaacggtacgtgcccctcgccgccccagcccctgtccccggccgccgccctgggcagcggcagcgccgccgcggtgcccaagatcgagagccccgacagcagcagcagcagcctgtccagcgggagcagccccccgggcagcctgccgtcggcgcggccgctcagcctggacggtgcggattccgcgccgccgccgcccgcgccctccgccccgccgccgcaccatagccagggcttcagcgtggacaacatcatgacgtcgctgcgggggtcgccgcagagcgcggccgcggagctcagctccggccttctggcctcggcggccgcgtcctcgcgcgcggggatcgcacccccgctggcgctcggcgcctactcgcccggccagagctccctctacagctccccctgcagccagacctccagcgcgggcagctcgggcggcggcggcggcggcgcgggggccgcggggggcgcgggcggcgccgggacctaccactgcaacctgcaagccatgagcctgtacgcggccggcgagcgcgggggccacttgcagggcgcgcccgggggcgcgggcggctcggccgtggacaaccccctgcccgactactctctgcctccggtcaccagcagcagctcgtcgtccctgagtcacggcggcggcggcggcggcggcgggggaggccaggaggccggccaccaccctgcggcccaccaaggccgcctcacctcgtggtacctgaaccaggcgggcggagacctgggccacttggcaagcgcggcggcggcggcggcggccgcaggctacccgggccagcagcagaacttccactcggtgcgggagatgttcgagtcacagaggatcggcttgaacaactctccagtgaacgggaatagtagctgtcaaatggccttcccttccagccagtctctgtaccgcacgtccggagctttcgtctacgactgtagcaagttttgacacaccctcaaagccgaactaaatcgaaccccaaagcaggaaaagctaaaggaacccatcaaggcaaaatcgaaactaaaaaaaaaaaatccaattaaaaaaaacccctgagaatattcaccacaccagcgaacagaatatccctccaaaaattcagctcaccagcaccagcacgaagaaaactctattttcttaaccgattaattcagagccacctccactttgccttgtctaaataaacaaacccgtaaactgttttatacagagacagcaaaatcttggtttattaaaggacagtgttactccagataacacgtaagtttcttcttgcttttcagagacctgctttcccctcctcccgtctcccctctcttgccttcttccttgcctctcacctgtaagatattattttatcctatgttgaagggagggggaaagtccccgtttatgaaagtcgctttctttttattcatggacttgttttaaaatgtaaattgcaacatagtaatttatttttaatttgtagttggatgtcgtggaccaaacgccagaaagtgttcccaaaacctgacgttaaattgcctgaaactttaaattgtgctttttttctcattataaaaagggaaactgtattaatcttattctatcctcttttctttctttttgttgaacatattcattgtttgtttattaataaattaccattcagtttgaatgagacctatatgtctggatactttaatagagctttaattattacgaaaaaagatttcagagataaaacactagaagttacctattctccacctaaatctctgaaaaatggagaaaccctctgactagtccatgtcaaattttactaaaagtctttttgtttagatttattttcctgcagcatcttctgcaaaatgtactatatagtcagcttgctttgaggctagtaaaaagatatttttctaaacagattggagttggcatataaacaaatacgttttctcactaatgacagtccatgattcggaaattttaagcccatgaatcagccgcggtcttaccacggtgatgcctgtgtgccgagagatgggactgtgcggccagatatgcacagataaatatttggcttgtgtattccatataaaattgcagtgcatattatacatccctgtgagccagatgctgaatagattttttcctattatttcagtcctttataaaaggaaaaataaaccagtttttaaatgtatgtatataattctcccccatttacaatccttcatgtattacatagaaggattgcttttttaaaaatatactgcgggttggaaagggatatttaatctttgagaaactattttagaaaatatgtttgtagaacaattatttttgaaaaagatttaaagcaataacaagaaggaaggcgagaggagcagaacattttggtctagggtggtttctttttaaaccattttttcttgttaatttacagttaaacctaggggacaatccggattggccctcccccttttgtaaataacccaggaaatgtaataaattcattatcttagggtgatctgccctgccaatcagactttggggagatggcgatttgattacagacgttcgggggggtggggggcttgcagtttgttttggagataatacagtttcctgctatctgccgctcctatctagaggcaacacttaagcagtaattgctgttgcttgttgtcaaaatttgatcattgttaaaggattgctgcaaataaatacactttaatttcagtcaaaaaaaaaaaaaaaaaaaaaaaaa

SEQ ID NO: 48 - protein, 553 aa, Homo sapiens
MQARYSVSSPNSLGVVPYLGGEQSYYRAAAAAAGGGYTAMPAPMSVYSHPAHAEQYPGGMARAYGPYTPQPQPKDMVKPPYSYIALITMAIQNAPDKKITLNGIYQFIMDRFPFYRDNKQGWQNSIRHNLSLNECFVKVPRDDKKPGKGSYWTLDPDSYNMFENGSFLRRRRRFKKKDAVKDKEEKDRLHLKEPPPPGRQPPPAPPEQADGNAPGPQPPPVRIQDIKTENGTCPSPPQPLSPAAALGSGSAAAVPKIESPDSSSSSLSSGSSPPGSLPSARPLSLDGADSAPPPPAPSAPPPHHSQGFSVDNIMTSLRGSPQSAAAELSSGLLASAAASSRAGIAPPLALGAYSPGQSSLYSSPCSQTSSAGSSGGGGGGAGAAGGAGGAGTYHCNLQAMSLYAAGERGGHLQGAPGGAGGSAVDNPLPDYSLPPVTSSSSSSLSHGGGGGGGGGGQEAGHHPAAHQGRLTSWYLNQAGGDLGHLASAAAAAAAAGYPGQQQNFHSVREMFESQRIGLNNSPVNGNSSCQMAFPSSQSLYRTSGAFVYDCSKF

SEQ ID NO: 49 - DNA, 1067 bp, Homo sapiens
mgtccgcgcaccaggaagctgaagaagaagaagaacgagaaggaggacaagcggccgcggaccgcgttcacggccgagcagctgcagagactcaaggcggagttccaggcaaaccgctacatcacggagcagcggcggcagaccctggcccaggaactcagcctcaacgagtcccagatcaagatctggttccagaacaagcgcgccaagatcaagaaagccacaggcatcaagaacggcctggcgctgcacctcatggcccagggactgtacaaccactccaccaccacggtccaggacaaagacgagagcgagtagccgccacaggccggggccgcgcccgcgccccctcccggcaccgccgccgtcgtctcccggcccctcgctgggggagaaagcatctgctccaaggagggagggagcgcagggaaaagagcgagagagacagaaagagagcctcagaatggacaatgacgttgaaacgcagcatttttgaaaagggagaaagactcggacaggtgctatcgaaaaataagatccattctctattcccagtataagggacgaaactgcgaactccttaaagctctatctagccaaaccgcttacgaccttgtatatatttaatttcaggtaaggaaaacacatacgtgtagcgatctctatttgctggacatttttattaatctcctttattattattgttataattattataattattataattattttatggccctcccccaccgcctcgctgcccccgcccagtttcgttttcgttgcctttttcatttgaatgtcattgcttctccggtgcctcccgacccgcatcgccggccctggtttctctgggacttttctttgtgtgcgagagtgtgtttcctttcgtgtctgcccacctcttctcccccacctcccgggtcccttctgtcggtctgtctgttctgcccccctttcgttttccggagacttgttgagaaatacgaccccacagactgcgagactgaaccgccgctacaagccaaagattttattatgttcagaaacctgtagtctgaaataaa

SEQ ID NO: 50 - protein, 392 aa, Homo sapiens
MEEQQPEPKSQRDSALGGAAAATPGGLSLSLSPGASGSSGSGSDGDSVPVSPQPAPPSPPAAPCLPPLAHHPHLPPHPPPPPPQHLAAPAHQPQPAAQLHRTTNFFIDNILRPDFGCKKEQPPPQLLVAAAARGGAGGGGRVERDRGQTAAGRDPVHPLGTRAPGAASLLCAPDANCGPPDGSQPAAAGAGASKAGNPAAAAAAAAAAVAAAAAAAAAKPSDTGGGGSGGGAGSPGAQGTKYPEHGNPAILLMGSANGGPVVKTDSQQPLVWPAWVYCTRYSDRPSSGPRTRKLKKKKNEKEDKRPRTAFTAEQLQRLKAEFQANRYITEQRRQTLAQELSLNESQIKIWFQNKRAKIKKATGIKNGLALHLMAQGLYNHSTTTVQDKDESE

SEQ ID NO: 51 - DNA, 2016 bp, Homo sapiens
ggcacgaggggcggcagccatcaggtaagccaagatgggtgcatacaagtacatccaggagctatggagaaagaagcagtctgatgtcatgcgctttcttctgagggtccgctgctggcagtaccgccagctctctgctctccacagggctccccgccccacccggcctgataaagcgcgccgactgggctacaaggccaagcaaggttacgttatatataggattcgtgttcgccgtggtggccgaaaacgcccagttcctaagggtgcaacttacggcaagcctgtccatcatggtgttaaccagctaaagtttgctcgaagccttcagtcccttgcagaggagcgagctggacgccactgtggggctctgagagtcctgaattcttactgggttggtgaagattccacatacaaattttttgaggttatcctcattgatccattccataaagctatcagaagaaatcctgacacccagtggatcaccaaaccagtccacaagcacagggagatgcgtgggctgacatctgcaggccgaaagagccgtggccttggaaagggccacaagttccaccacactattggtggctctcgccgggcagcttggagaaggcgcaatactctccagctccaccgttaccgctaatataagtaaagtttgtaaaattcatacttaataaacaatttaggacagtcatgtctgcttacaggtgttatttgtctgttaaaactagtctgcagatgtttcttgaatgctttgtcaaattaagaaagttaaagtgcaataatgtttgaagacaataagtggtggtgtatcttgtttctaataagataaacttttttgtctttgctttatcttattagggagttgtatgtcagtgtataaaacatactgtgtggtataacaggcttaataaattctttaaaaggagagaactgaaactagccctgtagatttgtctggtgcatgtgatgaaacctgcagctttatcggagtgatggcaatgctctgctggtttattttcaagtggctgcgttttttttagtttggcaggtgtagactttttaagttgggctttagaaaatctgggttagcctgaagaaaattgcctcagcctccacagtaccattttaaattcacataaaaggtgaaagctcctggttcagtgccatggcttcatggcattcagtgattagtggtaatggtaaacactggtgtgttttgaagttgaatgtgcgataaaattattagccttaagattggtaagctagcaatgaatgctagggtgggaagctggtgagccagtggccattagataaatacctttcaagtgtgagcttagacgtcaaccctaaaatacttaaccgtaatgctaattgtgatcattatgaatcccttcagtcacattagggggaaagtagttggctataagtacgtcattcttagtccagtcagtcttaaaaacatcttgggttacccactctgtccactcccataggctacagaaaaagtcacaagcgcatggtttccaaccatatgtgttttctgcagttatttctcttgttctggccaaacaaccctaaaaatccttaccattccacaaagttggaccatcacttgtgcacccactttgactatgagtataccaccacattgcatttctgtttgcaccatgtcttccaggagactagactactgttgtccagggtcaatttgagtgtaaagaaaatgtagacaaggaattgcccaattttaaattctgactttgctgacttaatttaaatgctcgttctgaaccaattttctcctatcttctctaggggtttcaaaagactcagttaattgatttccaggaagtactcatagcaagttcataaaagttcttgagacctaaatttcttcacaaaaaaagaaaagatcttaagtcatacattttaattgtgtagaggttgttcaactgaaggaataaatgtatattaaactaaaaaaaaaaaaaaaaaaaaaaaaaaaaa

SEQ ID NO: 52 - protein, 204 aa, Homo sapiens [sequence omitted]

SEQ ID NO: 53 - DNA, 3057 bp, Homo sapiens
gagcttctccggcggcgaggggatagctggttaccagaaggacttctttgcagggccagtggtttctgtcagattttcgccggtcacgactgctgaatatgagacgatctgcagcaccaagtcagttgcaggggaattccttcaaaaaaccaaaatttatacctccaggaagaagtaatccaggtctgaatgaagagattacaaaactgaatccagatataaaattatttgagggtgttgcaattaataacacctttctcccgtcacaaaatgatcttagaatatgcagtttaaatctgcctagtgaagaaagtactagagaaatcaataacagagataattgcagtggaaaatattgttttgaagcacctacactggcaacattagatccacctcatacagttcattcggctcctaaagaagtagcagtgtccaaggaacaagaagagaaatctgatagcctagttaaatatttcagtgttgtttggtgtaagccttcaaagaaaaaacataaaaagtgggaaggtgatgctgttcttattgtaaaaggaaagtcatttatattaaagaatttggaaggcaaagacattggaagaggcattggttataaattcaaagagcttgaaaagattgaagagggccaaacactgatgatttgtggaaaagaaatagaagtcatgggtgtaatctctccagatgacttcagcagtggcaggtgttttcagcttggaggaggaagtactgctatctcgcattcttctcaggttgccaggaaatgtttctctaaccctttcaaaagtgtttgtaaaccaagttcaaaggaaaatagacagaatgatttccaaaattgcaaaccacgccatgacccatatacgccaaattccctcgttatgccacgaccagataagaatcaccagtgggtattcaataagaactgtttccctcttgtggatgtagtgattgatccttaccttgtatatcatcttcgaccacatcagaaagaaggaatcatattcctttatgaatgtgtaatgggaatgagaatgaatggcagatgtggagctattcttgctgatgaaatgggtttagggaagacattgcaatgtatttcgctcatctggaccctgcagtgtcagggaccctatggaggcaagccagtaataaagaagacactaattgtcacacctggaagcttggtgaataattggaagaaagaatttcaaaaatggctaggaagtgaaaggatcaagatatttactgttgatcaggaccacaaagttgaagaattcatcaagtctatattttattctgttcttattatcagttatgaaatgttacttcgttccctggatcaaattaagaatataaaatttgatcttctaatctgtgacgaggggcatcgtttgaagaacagtgccattaagacaactacagccctcattagcctttcttgtgagaaaagaataattctaactggtactccaattcagaatgatctgcaagaattttttgcattaattgattttgtaaatccaggaatattaggctctttgtcatcttataggaaaatatatgaagaacccatcattttatcgagagaaccttctgcttctgaggaagaaaaggagttaggagaaagaagagcagctgaacttacttgcctcactggactctttatccttagaagaacccaagaaattataaataaatatctcccacctaaaatagagaatgttgtcttttgccgaccaggagcactacagattgagctttatcgaaagctgttaaattctcaggttgtcaggttctgccttcaagggttgttggaaaatagtccccatctaatatgtataggagctcttaaaaaactgtgcaatcacccctgccttttgttcaactctataaaggaaaaggaatgtagctcaacttgtgataaaaatgaagaaaagagtctatacaaaggcttgctaagtgtgtttcctgctgactacaaccctctcctgtttactgaaaaggagtcaggaaaactacaggtgttgtccaagctcttagcggttatccacgaacttcgacctactgaaaaggtggtgttggtatccaactatacacaaaccttgaacattttacaagaagtatgtaagcgtcatggatatgcttatacaagacttgatggacaaacaccaatctctcaaaggcagcagattgttgatggctttaacagtcaacactcttctttttttatttttttgttaagttcaaaagctggtggtgtaggacttaacctcattggaggatctcacttaattctctatgacattgattggaatccagccactgacattcaggcaatgtctagagtatggagagatggtcagaaatatcctgtacatatttacagactcctaactacaggtacaatagaagaaaagatctatcaaaggcagatcagtaagcaaggtctttgtggggcagttgtcgacctcaccaagacatctgaacatattcagttttcagtagaagaacttaaaaatttgttcacattacatgaaagttcagattgtgttactcatgatctgcttgactgtgagtgtacaggagaagaagttcatacaggtgattcgttggaaaaattcattgtctctagagattgtcagcttggtccacatcaccagaaatctaactccctgaaacctctttctatgtcccagctgaagcaatggaaacatttttctggagatcatttaaatcttacagatccttttcttgaaagaataacagaaaatgtgtcattcatttttcagaatataaccactcaagctactggcacatagtgaaagattacttctgacattccattgctcttcttttgaaaattagtatggtaattaaatgtactttttgaaaattaatagaattatttaaattacagtatatgttgcaaaatatatcacttttgatacaatagtcaaaattgagtggtttaatgttttgtaaatattaagtgtttaaatgaaaaataaagatgtgcttatatcattgtaaaaaaaaaaaaaaaaa

SEQ ID NO: 54 - protein, 910 aa, Homo sapiens
MRRSAAPSQLQGNSFKKPKFIPPGRSNPGLNEEITKLNPDIKLFEGVAINNTFLPSQNDLRICSLNLPSEESTREINNRDNCSGKYCFEAPTLATLDPPHTVHSAPKEVAVSKEQEEKSDSLVKYFSVVWCKPSKKKHKKWEGDAVLIVKGKSFILKNLEGKDIGRGIGYKFKELEKIEEGQTLMICGKEIEVMGVISPDDFSSGRCFQLGGGSTAISHSSQVARKCFSNPFKSVCKPSSKENRQNDFQNCKPRHDPYTPNSLVMPRPDKNHQWVFNKNCFPLVDVVIDPYLVYHLRPHQKEGIIFLYECVMGMRMNGRCGAILADEMGLGKTLQCISLIWTLQCQGPYGGKPVIKKTLIVTPGSLVNNWKKEFQKWLGSERIKIFTVDQDHKVEEFIKSIFYSVLIISYEMLLRSLDQIKNIKFDLLICDEGHRLKNSAIKTTTALISLSCEKRIILTGTPIQNDLQEFFALIDFVNPGILGSLSSYRKIYEEPIILSREPSASEEEKELGERRAAELTCLTGLFILRRTQEIINKYLPPKIENVVFCRPGALQIELYRKLLNSQVVRFCLQGLLENSPHLICIGALKKLCNHPCLLFNSIKEKECSSTCDKNEEKSLYKGLLSVFPADYNPLLFTEKESGKLQVLSKLLAVIHELRPTEKVVLVSNYTQTLNILQEVCKRHGYAYTRLDGQTPISQRQQIVDGFNSQHSSFFIFLLSSKAGGVGLNLIGGSHLILYDIDWNPATDIQAMSRVWRDGQKYPVHIYRLLTTGTIEEKIYQRQISKQGLCGAVVDLTKTSEHIQFSVEELKNLFTLHESSDCVTHDLLDCECTGEEVHTGDSLEKFIVSRDCQLGPHHQKSNSLKPLSMSQLKQWKHFSGDHLNLTDPFLERITENVSFIFQNITTQATGT

SEQ ID NO: 55 - DNA, 9231 bp, Homo sapiens
cttgagtgttagagctgagtagttttcccagaatctctaagtcctttttatgctcttttatgaatgaatagaattagtaaaagataaataaattttttcttttggatttcttaaccagtggaaaaaatgttgactttaaaagttcataaaatcaaattttgcttaagaatatgttatttccacttgtgaggccagcctggtagacctctgggatccttttctgttcactcacacaccactgagataaggagtgaagtgtgggctaaatagggctgaggcttgggcaagggcatttctgccagagcaccagagacgtcagcatctcaagggcactgtggtatggaaaaggacgccacatgagtaaattttaaaaatataaatattttaaagggtaaaaatgagggtccctgtatttgaggatataaaagatgaaactgaagaagaaaagataggggaagaagaaaatgaagaagaccaggtcttctataagcctgttattgaagacttaagcatggaattggccagaaaatgcacggaactcattagcgatatccgttataaagaagagtttaaaaagtccaaggataagtgtacatttgtgactgacagtcctatgctaaaccatgtaaaaaatatcggtgcttttatttctgaggcaaaatacaaaggcaccattaaagctgacctttctaattctctttataagcggatgccagccacaattgacagtgtttttgcaggagaagttacacagctccagagtgaggtggcctacaagcagaaacatgatgctgccaaaggattctcagattatgcccacatgaaggagccccctgaggttaaacatgccatggaggtcaataaacaccagagtaatatttcttataggaaagacgtgcaggacacccacacgtacagtgcagaacttgaccgaccagacatcaagatggcaacccagatctctaagatcataagcaatgcagaatacaagaaaggacaaggaataatgaataaagagcccgctgtaattggaagaccagattttgaacatgccgtggaagcttctaaactttctagtcaaattaaatacaaagaaaaatttgataatgaaatgaaggataagaaacatcattacaatcctcttgaaagtgcttcttttaggcagaatcagcttgctgctacactggcgagcaatgtgaagtacaagaaagacattcaaaatatgcatgatccagtttcagatctcccaaatttgttgtttttagaccatgttttgaaagccagcaaaatgctcagcggccgagaatataaaaagctctttgaggaaaacaaaggaatgtatcattttgatgcagatgctgtggaacatctgcaccataaaggcaatgccgtcctccaaagtcaggtgaaatataaagaagaatatgagaaaaataagggaaagccaatgcttgaatttgttgagacaccatcatatcaagcttcaaaggaggctcaaaagatgcaaagtgaaaaagtttacaaagaggattttgagaaggagattaaaggaaggtcatcactggatttagacaagactccagaatttttacatgtaaagtacatcaccaaccttctgagggagaaagaatataaaaaagatttggaaaatgagataaaagggaaaggaatggaacttaattcagaagttcttgatatccaaagagcaaagcgagcctctgaaatggcaagtgagaaagaatacaagaaagacctggagtcaataattaaagggaaaggaatgcaagctggcactgacacccttgaaatgcagcatgccaagaaggctgcagagatagcgagtgagaaagactataaaagagatctggagactgaaattaaagggaaagggatgcaggtgagcacagacactcttgatgtccagagagctaagaaagcatccgagatggccagccagaaacaatacaagaaggacttagaaaatgaaattaaagggaaaggaatgcaagtgagcatggatatcccagatatccttcgagccaagaggacatctgaaatctatagccagagaaagtataaagatgaagcagagaagatgctttctaactattctaccatagcagatactcctgaaattcagagaattaagacaactcaacaaaacattagtgcggtattttataagaaagaagtgggagctggcactgcagtgaaagatagcccagagatcgaacgagtgaagaaaaatcagcagaatattagttcagtgaaatacaaagaagagattaaacatgcaacagccatttctgatcctccagaactaaagagagttaaagaaaaccagaagaacatcagcaatctccagtataaagagcaaaactacaaggccactccggtaagcatgaccccggagatagagagagtgaggcgaaaccaggagcagctgagtgcggtaaaatataagggagaacttcaacggggaactgcaatttctgatccaccagagctgaagagggcaaaagaaaaccagaaaaacatcagcaatgtttattacagaggtcagctgggaagagctaccactttaagtgtaactcctgaaatggaaagagtgaagaagaatcaagaaaatattagctcggtaaaatatacccaggaccataaacagatgaaaggtagaccaagtctgattttagatacacctgctatgagacatgttaaagaagcacaaaatcatatttcaatggtaaaataccatgaagattttgaaaaaacaaaggggagaggctttactcccgtcgtggacgatcctgtgacagagagagtgaggaagaacacccaggtggtcagcgatgctgcctataaaggggtccaccctcacatcgtggagatggacaggagacctggaatcattgttgacctcaaagtttggcgcacagatcctggctccatcttcgaccttgatcccctggaagacaatattcagtctagaagtctccatatgctctctgaaaaggcgagtcactataggcgacactggtctcgatcccattccagcagtactttcggtacaggtctcggagacgacaggtcagaaatctccgagatttaccctagcttttcatgctgcagtgaggtaacaagaccgtctgatgaaggagcacctgttcttcccggagcctatcagcaaagccattcccaaggctatggctacatgcaccagaccagtgtgtcatccatgagatcaatgcagcattcaccaaatctaaggacctaccgagccatgtacgattacagtgcccaggatgaagacgaggtctcctttagagacggcgactacatcgtcaacgtgcagcctattgacgatggctggatgtacggcacagtgcagagaacagggagaacaggaatgctcccagcgaattacattgagtttgttaattaattatttctccctgccctttgagctttattctaatgtatcccaaacctaatctttttaaaagatagaagatacttttaagacaacttggccattattttacaatgatgtatccttcctttgacaattagacacacaggtaccaggaagaaggaatgacctctgggctgaaaacagcagcattttcagtaattcctacaaacaaaaatctttgtgtctggacacctggtgctgctaattgtgttcatggtttcctttgattggctattgaacccttctgggaaatgtatttttgtagactttaatagagaagttgattgtcccttaaatgtagtgtgtgtttgaaacttcttagctgtcactttggaatcaccccaagccaattctcttaactctgtaatgcagccaataatttcaaacccgttttgcttttgagtcatgaggcaatttccaatattagtgaaaattgcccaatataataagtgtaaacagtggcagaaggacagtctggttaaaattatattgactggtggccttagggatctagaaacttctactaaacagagaaatttccttgttccctaggctgactggtatctatttatttctcatttgtaccaaggcatctcctactctccatttatattctatggacccaagtctatgctcagttccacagaatgtcaggaccaaataacttcacagctactctgcaaagggcaaattataatgtcattgatataatttccctagtagcatttaccctgttgcatgtcatgtagattcaagcttctgtaacataggcagctgcactgcgcgttcctattattgaagcaaaaagggtgactgatacctaaaagccctttcttcctctagtcgccagctcatcagaaaaacatactttgaaaagatgcttgagattttcctgctgcatcgcactctagttttgaaggatttacatcttaggaaataacatgtatactctagtaaataagcgatttaggtgttccattgaacagctttgattaacttaatgccaccattgatttcaaagtgaagaaaatgtaacagaagccagtgaagcaatggaagctggagtgtgactggaaaaatactcagcaaacaaagttaccaattccatacagagatgatctggtatcttcttttggaaaatggtattcaaattctggaatggaaatctagccaccaaaacgggttaatcaaaagacgtccttttccatttttttttgcttttattttctaaatcatttttaagggaatgaaacaggaatgtcatcagagattttttagtacaggcccaagagcctgttctctaagaaagaaattgttgccatgttttgattttcgaataagtgactttgcaggctttatgctagcccttgctggtgggtcttgaaatttcatccagagtctgcagtccaggtcaccaagccagcggcacccgtcggcaaccctgtgtttttctgattgtgccgtttactgtgacctgcaacggggtggcattcacttagggtctgacttcacagctatgacaaaaccgaaaaagcaaaactgcaaaaaagtactaagatgtacgggtcttggggatatctgccttatatgttatattcaaggaaattaacaaaacatcctgtaaaacatcgtttaaggaaacgtttactagtccaaaggccaaagctaatttatttccactttagaaaagttagcacatgcttttgaaaatctgtgatttcattttattaggctaaaagggtaaataggctttattacactgaagctgcatctatatgtcactgacataaagttgaaaaaataaatgcaggcaaataactagagacttcttttaagggggtttggctggttttctctcactgaaatggccagtcgtgattaaagtgataaaaccccatatctgttttggtatattgtacacaaacctacaaaaataaactgaacttgcaatatttttgcaaaaaaatctgtcgttaaaactgaggataaaatacctgctcaattttattttactaagtatatatttacatttcacccaggcaggccattttcttttgtgattataagaaagagtagttgttgattaaattttcagactaaatataggacaggtacaattttggataaatagcacatttataagaaccgcaatgaaaactgacttgaaataatgcttgtaatcaggaaagtaatttcatccaccgatttcaaaaccagattcactgagcataaaagtcaatacatatttgaggaataagtctcctaaaattttaagcttcacgtaataatgtttgcatagcaaaatatttctgcttcaagcctttaggaattaagatctgatcagaatttaactaaagggtagttgttttacaatgaagactaaaactgaacaagatgttgcatgctcttgaggccataatttggtagtgttggcagttgttaataaagcttgtcaggatgttaagcatctcaggagaaatattggaaaattatatgtataaaaccaaagtgctatttttaaaagcatcatttaaaaaaaaatgacatgcctgaacaacttttccactttccacgtgcttccctcccacctttggtttggcaacaggtatctcgtgcatgaagctgacagctaaagaagattttaaaaattgagttaaagatgactgtgtaaatgtccaagcacagagagcatgcacctgactttctaaagtttgatgtgttctcaagcctgacagaagcacaaggaacagtttgatacacttttaaaaggttctgaaaacaaagctgtatagggatcctctctctcttgagcaaagtatagcaacagaatatattgcttttgttgtaagcttttgtagtacatgtttttactaataattcttgttctctagaaagctttctatttctaacctatggcaaaatgaatccttcatgtcttcttgttattgtttacacacttgcagtgtagcccagtttgaaatatttatttggttatcaactgcccatggaggaggctcttgatgatcccaggtctcctcgacctccatacaccacacaggcatttgtaagcacagtttccacaagcaccttgtaggaatatggataagattagaccagcccctctctgtccactgggtttatttcttgaagaagatgcagatctggtttttccaatgtgccacagtctttccttatcctctccatgctgagcttgacaacactctgggaatgaggaacaagactttttctaaaaagatagtggaagttcaagggatgtacctcgttttcaggttcatccatctccagtggaatgttttcaataaaagatgaagaaaatgtgtgtgatctttaataacacatccctatagaaagtggataaaagatataccaaaactgtaatacagatatatacaaatataggtgcctttttgattactcttgtttgtctagtatgctcttggaaagaaaaccaagcaagcaagttgctgcctattctatagtaatattttattacacatgattgatatttttgtggtagggaagtgggatgctcctcagatattaaaggtgttagctgattgtattttatctctaaagatttagaactttagaaaatgccgacttcttccatctatttctgaaaggttctttgtggatttatatagagttgagctatataaacattaactttagatttgggatttaaaatgcctattgtaagatagaataattgtgaggctggattcactacacaagatgaacttcacttcataaattaattataccttagcgatttgcttctgataatctaaaagtggctagattgtggttgttttggttaaggtgatatggaggtgggagagcttttagttaagtaagaagctatgtaaactgacaaggatgctaaaataaaagtctctgaagtattccatgccttttggaccctttcctcgcaactaactgtcaactgttgatcaaaaaagtcaaggcattgtatgttgcttctgtggttattattctgtgatgcttagactacttgaacccataaacttggaagaatctttgagcaaattttctcagttgtctgtatgacttcagtatattcctgggaatgccataggattttttgtgcttgatacatggtatccagtttgcatagtatcacttctttgtaatccagttgctgttaagaatgatgtactttaaaggaaaagagaaaactgcatcacagtcccattctccagtgtccatgcaatgaattgctgagcatttaggaagcagcaccaagtctattacaggcatggtgtgaaacttgatgtttgacctgtgatcaaaattgaaccattgtacagtttggcttctgtttgcttcaaaatatgtagaattgtggttgatgattaatttgcgagactaactttgagagtgtaacagttttgaagaaaacattgaatgttttgcaaatgaaggggcttcacggaatgttacaatgttactaatataatttggcttttgttatgcaaattgttaacaccagctattaaaatatattttagtagaaatgctttaattcatatttttttcctctacactgtgaatctttaagccttggtggactagagcaacatcgtgctgcccaaaggactaacctatgcaaactagttcacattttagtggatgtcgcagttaatgtgtaataagacattatttcccctgcataatgtacaacagcattgaaatgacacattaagcctagcatcacattgtatagtacagtcactcacaaacccttcaaggctaccctaatcattaacattaatatttgtttaaaagcaaatcaccgatttatctattgaaactacttaaatgacggcaaaccaggaatgacagatggctgtgtcagcaatggctttaatgtgttccctgcaagtggtctcctatgatagaactgcgttctcaaatgcactctcttcagggtcttaatattctgtgttttctctctgtatttgtaaaacattataacacattaatttcctatctctacacatttggtttgcttaaataaatgcaggatataaaaaaaatggttcacttcttggctctcaccgtggtttcttggagcatgggttgttagatgcaagcaatgcaccctaataataccccgggtctgagatttaacatgacaactcacatcaaatcgcatcagaggtgtgtgctgccttcagtgcatttacattggtgaatcagtcaagatattttcctcccccaaataaacttagttgtaagtgataacaatattatgcttctccaagctcagtatctttctgattttatatcaaagtaccgcaacaatgcatcattgtagttaatttatttcaagaataaattcctcatatgtcctcaatagtacaattctaattttcttctattcataagatgaaagaaatggtttggagcatagaatagaaagtgcacaaattgagtacataaaatgggaagcaactgatttctcagctaagaaaggctcatttatcacagaacacaattgcttttctccccccactacgcttcccataattgaaaaagtgagtccctatttttcacactcatataaatctatgcgatttggatgctagtcttattgtattattttgtaaaactttctctttggctcataatccttcctaattgtaaattgataaactttgcggatgacatctgctcgtagaataaacacttcttccaaaaaaaaaaaaaaaaaa

SEQ ID NO: 56 - protein, 1014 aa, Homo sapiens
MRVPVFEDIKDETEEEKIGEEENEEDQVFYKPVIEDLSMELARKCTELISDIRYKEEFKKSKDKCTFVTDSPMLNHVKNIGAFISEAKYKGTIKADLSNSLYKRMPATIDSVFAGEVTQLQSEVAYKQKHDAAKGFSDYAHMKEPPEVKHAMEVNKHQSNISYRKDVQDTHTYSAELDRPDIKMATQISKIISNAEYKKGQGIMNKEPAVIGRPDFEHAVEASKLSSQIKYKEKFDNEMKDKKHHYNPLESASFRQNQLAATLASNVKYKKDIQNMHDPVSDLPNLLFLDHVLKASKMLSGREYKKLFEENKGMYHFDADAVEHLHHKGNAVLQSQVKYKEEYEKNKGKPMLEFVETPSYQASKEAQKMQSEKVYKEDFEKEIKGRSSLDLDKTPEFLHVKYITNLLREKEYKKDLENEIKGKGMELNSEVLDIQRAKRASEMASEKEYKKDLESIIKGKGMQAGTDTLEMQHAKKAAEIASEKDYKRDLETEIKGKGMQVSTDTLDVQRAKKASEMASQKQYKKDLENEIKGKGMQVSMDIPDILRAKRTSEIYSQRKYKDEAEKMLSNYSTIADTPEIQRIKTTQQNISAVFYKKEVGAGTAVKDSPEIERVKKNQQNISSVKYKEEIKHATAISDPPELKRVKENQKNISNLQYKEQNYKATPVSMTPEIERVRRNQEQLSAVKYKGELQRGTAISDPPELKRAKENQKNISNVYYRGQLGRATTLSVTPEMERVKKNQENISSVKYTQDHKQMKGRPSLILDTPAMRHVKEAQNHISMVKYHEDFEKTKGRGFTPVVDDPVTERVRKNTQVVSDAAYKGVHPHIVEMDRRPGIIVDLKVWRTDPGSIFDLDPLEDNIQSRSLHMLSEKASHYRRHWSRSHSSSTFGTGLGDDRSEISEIYPSFSCCSEVTRPSDEGAPVLPGAYQQSHSQGYGYMHQTSVSSMRSMQHSPNLRTYRAMYDYSAQDEDEVSFRDGDYIVNVQPIDDGWMYGTVQRTGRTGMLPANYIEFVN

SEQ ID NO: 57 - DNA, 4197 bp, Homo sapiens
ctgcttctcccccgccccatcatctccagcgcgtggtgggggaactgctggggaaggcgattggcatcgatctctccatcccttccgaggcccgacttcggaattgtcttagaagaaagaaggcaagccaccattttacccacgtaaatatatgaatatatttctgacattgaggtgttccagaagatgataaagaaatgatagcagctccagaaataccaactgattttaatctactacaggagtcagaaacacatttttcttctgacacagattttgaagatatcgaaggaaaaaaccaaaagcaaggcaaaggcaaaacttgtaaaaaaggcaaaaagggcccagcagaaaagggcaaaggtggaaatggaggaggaaaacctccttctggtccaaaccgaatgaatggtcatcaccaacagaatggagtggaaaacatgatgttgtttgaagttgttaaaatgggcaagagtgctatgcagtcggtggtagatgattggatagaatcatacaagcatgaccgagatatagcacttcttgaccttatcaacttttttattcagtgttcaggctgtaaaggagttgtcacagcagaaatgtttagacatatgcagaactctgagataattcgaaaaatgactgaagaattcgatgaggatagtggagattatccacttaccatggctggtcctcagtggaagaagttcaaatccagtttttgtgaattcattggcgtgttagtacggcaatgtcaatatagtatcatatatgatgagtatatgatggatacagtcatttcacttcttacaggattgtctgactcacaagtcagagcatttcgacatacaagcaccctggcagctatgaagttgatgacagctttggtgaatgtggcactaaatcttagcattaatatggataatacacaaagacaatatgaagcagaacggaataaaatgattggaaaacgagccaatgagaggctagaactcctgctacaaaagcggaaagagcttcaggaaaatcaagatgaaatagaaaatatgatgaatgcaatatttaaaggagtgtttgtacatagataccgtgatgcgatagctgaaattcgagctatttgcattgaagagattggcatttggatgaagatgtatagtgatgcctttcttaatgacagttatttaaaatatgttggttggactatgcatgataagcaaggtgaagtaagactcaaatgtcttactgctctacaagggctttattataacaaagagcttaattccaaactggaactttttaccagtcggttcaaggatagaattgtgtctatgacccttgacaaagaatatgatgttgcagtacaagcaataaaattactcactcttgttttacagagtagtgaagaagttctcactgcagaagattgtgaaaatgtctatcatctggtttattcagctcaccggccagtagcagtagcagctggagaatttctctacaaaaagctcttcagtcgtagagatccagaggaggatggaatgatgaaaagaagaggaagacaaggtccaaatgccaaccttgttaagacattggtttttttctttctagaaagtgagttacatgagcatgcagcataccttgtggatagcatgtgggactgtgctactgagctgctgaaagactgggaatgtatgaatagcttgttactggaagagccacttagtggagaggaagcactaacagataggcaagagagtgctctgattgaaataatgctttgtaccattagacaagcggctgaatgtcatcctcccgtgggaagagggacaggaaaaagggtgcttacagcaaaggagaagaagacacagttggatgataggacaaaaatcactgagctttttgccgtggcccttcctcagttattagcaaaatactctgtagatgcagaaaaggtgactaacttgttgcagttgcctcagtactttgatttggaaatatataccactggacgattagaaaagcatttggatgccttattgcgacagatccggaatattgtagagaagcacacagatacagatgttttggaagcatgttctaaaacttaccatgcactctgtaatgaagagttcacaatcttcaacagagtagatatttcaagaagtcaactgatagatgaattggcagataaatttaaccggcttcttgaagattttctgcaagagggtgaagaacctgatgaagatgatgcatatcaggtattgtcaacattgaagaggatcactgcttttcataatgcccatgacctttcaaagtgggatttatttgcttgtaattacaaactcttgaaaactggaatcgaaaatggagacatgcctgagcagattgttattcacgcactgcagtgtactcactatgtaatcctttggcaacttgctaagataactgaaagcagctctacaaaggaggacttgctgcgtttaaagaaacaaatgagagtattttgtcagatatgtcaacattacctgaccaacgtgaatactactgttaaggaacaggccttcactattctgtgtgatattttgatgatcttcagccatcagattatgtcaggagggcgtgacatgttagagccattagtgtatacccctgattcttcattgcagtctgagttgctcagctttattttggatcatgtcttcattgaacaggatgatgataataatagtgcagatggtcagcaagaggatgaagccagtaaaattgaagctctgcacaagagaagaaatttacttgcagcattttgtaagctaattgtatatactgtggtggagatgaatacagctgcagatatcttcaaacagtatatgaagtattataatgactatggagatatcatcaaagaaacaatgagtaaaacaaggcagatagacaaaattcagtgtgctaagacccttattctcagtctgcaacagctttttaatgaaatgatacaagaaaatggctataattttgatagatcatcctctacatttagtggcataaaagaacttgctcgacgttttgctttaacttttggacttgatcagttgaaaacaagagaagccattgccatgctacacaaagatggcatagaatttgcttttaaagagcctaatccgcaaggggagagccatccacctttaaatttggcatttcttgatattctgagtgaattttcttctaaactacttcgacaagacaaaagaacagtgtatgtttacttggaaaagttcatgacctttcagatgtcactccgaagagaggatgtgtggcttccactgatgtcttaccgaaattctttgctagctggtggtgatgatgacaccatgtcagtcattagtggaatcagcagccgggggtcaacagtacggagtaaaaaatcaaaaccatctacaggaaaacggaaagtggttgagggcatgcagctttcactcactgaagaaagtagtagtagtgacagtatgtggttaagcagagaacaaacactgcacacccctgttatgatgcagacaccacaactcacctccactattatgagagagcccaaaagattacggcctgaggatagcttcatgagtgtttatccaatgcagactgaacatcatcaaacacctcttgattataatcggcgtggcacaagcctaatggaagatgatgaagagccaattgtggaagatgttatgatgtcctcagaagggaggattgaggatcttaatgagggaatggattttgacaccatggatatagatttgccaccatcaaagaacagacgagagagaacagaactgaagcctgatttctttgatccagcttcaattatggatgaatcagttcttggagtgtcaatgttttaataccagtacacaattaaatctgtggtgaagtcattttctaagtggaagaggaaattttaaagtgtggtagatacagtgaaattctgtacagatttttctctaaggagaatatgacatgcttatgcttaccaagatcaagtgcattgaggggcagttttgtttgcctgaataaacgtaaaggacaagtaaacaatttgatgataagctacagtttttcttagaaagtaaatattttatttatgcgctgttagttggcttttgaatcgattatttcatgcttttttttaaaaaaaaaaaaaaaaaa

SEQ ID NO: 58 - protein, 1231 aa, Homo sapiens
MIAAPEIPTDFNLLQESETHFSSDTDFEDIEGKNQKQGKGKTCKKGKKGPAEKGKGGNGGGKPPSGPNRMNGHHQQNGVENMMLFEVVKMGKSAMQSVVDDWIESYKHDRDIALLDLINFFIQCSGCKGVVTAEMFRHMQNSEIIRKMTEEFDEDSGDYPLTMAGPQWKKFKSSFCEFIGVLVRQCQYSIIYDEYMMDTVISLLTGLSDSQVRAFRHTSTLAAMKLMTALVNVALNLSINMDNTQRQYEAERNKMIGKRANERLELLLQKRKELQENQDEIENMMNAIFKGVFVHRYRDAIAEIRAICIEEIGIWMKMYSDAFLNDSYLKYVGWTMHDKQGEVRLKCLTALQGLYYNKELNSKLELFTSRFKDRIVSMTLDKEYDVAVQAIKLLTLVLQSSEEVLTAEDCENVYHLVYSAHRPVAVAAGEFLYKKLFSRRDPEEDGMMKRRGRQGPNANLVKTLVFFFLESELHEHAAYLVDSMWDCATELLKDWECMNSLLLEEPLSGEEALTDRQESALIEIMLCTIRQAAECHPPVGRGTGKRVLTAKEKKTQLDDRTKITELFAVALPQLLAKYSVDAEKVTNLLQLPQYFDLEIYTTGRLEKHLDALLRQIRNIVEKHTDTDVLEACSKTYHALCNEEFTIFNRVDISRSQLIDELADKFNRLLEDFLQEGEEPDEDDAYQVLSTLKRITAFHNAHDLSKWDLFACNYKLLKTGIENGDMPEQIVIHALQCTHYVILWQLAKITESSSTKEDLLRLKKQMRVFCQICQHYLTNVNTTVKEQAFTILCDILMIFSHQIMSGGRDMLEPLVYTPDSSLQSELLSFILDHVFIEQDDDNNSADGQQEDEASKIEALHKRRNLLAAFCKLIVYTVVEMNTAADIFKQYMKYYNDYGDIIKETMSKTRQIDKIQCAKTLILSLQQLFNEMIQENGYNFDRSSSTFSGIKELARRFALTFGLDQLKTREAIAMLHKDGIEFAFKEPNPQGESHPPLNLAFLDILSEFSSKLLRQDKRTVYVYLEKFMTFQMSLRREDVWLPLMSYRNSLLAGGDDDTMSVISGISSRGSTVRSKKSKPSTGKRKVVEGMQLSLTEESSSSDSMWLSREQTLHTPVMMQTPQLTSTIMREPKRLRPEDSFMSVYPMQTEHHQTPLDYNRRGTSLMEDDEEPIVEDVMMSSEGRIEDLNEGMDFDTMDIDLPPSKNRRERTELKPDFFDPASIMDESVLGVSMF

SEQ ID NO: 59 - DNA, 942 bp, Homo sapiens
gaattccgctccgcactgctcactcccgcgcagtgaggttggcacagccaccgctctgtggctcgcttggttcccttagtcccgagcgctcgcccactgcagattcctttcccgtgcagacatggcctctggcaccaccaccaccgccgtgaagattggaataattggtggaacaggcctggatgatccagaaattttagaaggaagaactgaaaaatatgtggatactccatttggcaagccatctgatgccttaattttggggaagataaaaaatgttgattgcatcctccttgcaaggcatggaaggcagcacaccatcatgccttcaaaggtcaactaccaggcgaacatctgggctttgaaggaagagggctgtacacatgtcatagtgaccacagcttgtggctccttgagggaggagattcagcccggcgatattgtcattattgatcagttcattgacaggactatttgccacgacatttcaaaggattccaagagagaatattggtgtccatgctgtgatgattcctcagctcctctcatctgatctccgtcctggcccccatgactttctttgcggtagttagggtgtggtatgtgccactgaggcccacacctattggcaatttatagcactgatctgtcatcaataccacttgctgtcttggatgtgaagatgatttttcctgcagggattccctctacaaaattaaaaacactgggcatgtggaaataatattcacgctttaaattgtcttttctattcactacaccaggggtccccgacccctaggcaacagactgtggccctagtgtagtgaatagaaaagacaatttaaagcatgaatattatttcctcatgcccagtgtttttaattttggtactggtctgtggcttgttagaaaccaggctgcacagcagaaggtgggcagcag

SEQ ID NO: 60 - protein, 143 aa, Homo sapiens [sequence omitted]

(19) United States
(12) Patent Application Publication
(10) Pub. No.: US 2011/0183336 A1
(43) Pub. Date: Jul. 28, 2011

(54) Method to Predict Responsiveness of Breast Cancer to Polyamine-Type Chemotherapy

(75) Inventors: JOE W. GRAY, SAN FRANCISCO, CA (US); DEBOPRIYA DAS, ALBANY, CA (US); WEN-LIN KUO, SAN RAMON, CA (US); NICHOLAS J. WANG, BURLINGAME, CA (US); RICHARD M. NEVE, SAN MATEO, CA (US); PAUL T. SPELLMAN, BENICIA, CA (US); JANE FRIDLYAND, BERKELEY, CA (US); KOEI CHIN, FOSTER CITY, CA (US); ZHI HU, EL CERRITO, CA (US)

(73) Assignee: THE REGENTS OF THE UNIVERSITY OF CALIFORNIA, OAKLAND, CA (US)

(21) Appl. No.: 12/767,725

(22) Filed: Apr. 26, 2010

Related U.S. Application Data
(63) Continuation of application No. PCT/US2008/081385, filed on Oct. 27, 2008.
(60) Provisional application No. 61/000,477, filed on Oct. 26, 2007.

(51) Int. Cl. C12Q 1/68; G01N 33/573; G01N 33/53; C07H 21/02; C07H 21/00; C07K 16/32

(52) U.S. Cl. 435/6.12; 435/6.14; 435/7.1; 436/501; 536/24.5; 530/387.7

(57) ABSTRACT

Methods of-identifying a basal or luminal phenotype of a cell, comprising detecting expression of one or more of a set of predictive biomarker genes or proteins that identify the cell as having a basal or luminal cancer subtype and compositions for treating identified basal or luminal cancers.

### CROSS-REFERENCE TO RELATED APPLICATIONS

Priority is hereby claimed to U.S. provisional application Ser. No. 61/000,477, filed 26 Oct. 2007, which is incorporated herein.

### STATEMENT OF GOVERNMENTAL SUPPORT

The invention described and claimed herein was made in part under Work for Others Agreement LB7003395 with Progen Pharma, under Grant No. U54 CA112970 awarded by the National Institutes of Health/National Cancer Institute and under Contract No. DE-AC02-05CH11231 awarded by the U.S. Department of Energy. The United States Government has certain rights in this invention.

### REFERENCE TO SEQUENCE LISTING

This application hereby incorporates by reference the attached sequence listing in paper and computer readable form.

### FIELD OF THE INVENTION

The present invention relates generally to genetic markers involved predicting the responsiveness of cancers, especially breast cancers, to polyamine-type chemotherapeutic agents.

### BACKGROUND

It has been known since the 1950's that conformation is a determinant in the spatial arrangement of functional groups, and that enzymes or drug receptors prefer specific ligand conformations or specific distributions of conformations. This fruitful concept led to many decisive successes in drug design. A few examples will suffice to illuminate the subject.

The synthesis of conformationally constrained analogs of an inherently conformationally flexible substance such as acetyl choline helped to elucidate its “bioactive conformations,” i.e., those conformers which are active at the muscarinic and nicotinic receptors. The trans-cyclopropyl analog of acetyl choline was shown to be preferred by the muscarinic receptor. Conformationally restricted analogs of dopamine, GABA, glutamic acid, histamine and serotonin have been obtained by introducing rigid rings into their structures. The constrained analogs have valuable chemotherapeutic effects.

The use of conformational restriction has also been very fruitful in the design of bioactive polypeptides. Polypeptides have so many flexible torsion angles that enormous numbers of conformations are possible in solution. The introduction of rings into the linear peptide chains reduces the number of conformations and has allowed the preparation of several biologically active substances. For instance, a cyclic hexapeptide possessing somatostatin activity is known. Conformationally restricted enkephalin analogs are known, as are bicyclic lactam inhibitors (enalapril and enalaprilat) of the angiotensin-converting enzyme.

Similar strategies have led to the development of a peptidomimetic benzodiazepine containing at least two conformational restrictions: a bicyclic heterocycle and an acetylene linker. The benzodiazepine is a non-peptide RGD (Arg-Gly-Asp) receptor antagonist.

The concept of conformational restriction led to the discovery that the bioactive conformation of the immunosuppressor cyclosporin A (CsA) only binds to cyclophylin A when the amide bond between the 9-position and 10-position residues in CsA is trans.

These discoveries have yielded new chemotherapeutic agents for treating a host of cancers, and have resulted in marked improvements in long-term survival prospects for cancer sufferers. However, many forms of cancer, most notably breast cancers, remain recalcitrant to treatment with drugs.

Breast cancer is one of the most common malignancies among women and shares, together with lung carcinoma, the highest fatality rate of all cancers affecting females. The current treatment of the breast cancer is limited to a very invasive, total or partial mastectomy, radiation therapy, or chemotherapy, the latter two resulting in serious undesirable side effects.

It is now well established that breast cancers progress through accumulation of genomic and epigenomic aberrations that enable development of aspects of cancer pathophysiology such as reduced apoptosis, unchecked proliferation, increased motility, and increased angiogenesis. Discovery of the genes that contribute to these pathophysiologies when deregulated by recurrent aberrations is important to understanding mechanisms of cancer formation and progression and to guide improvements in cancer diagnosis and treatment.

Analyses of expression profiles have been particularly powerful in identifying distinctive breast cancer subsets that differ in biological characteristics and clinical outcome. However, there remains a paucity of data that matches distinctive breast cancer subsets with chemical agents that are specifically effective against the cancer subset.

Thus, there remains a long-felt and unmet need to match the specific type of breast cancer to a chemical agent(s) most likely to inhibit the further progression of the cancer.

### SUMMARY OF THE INVENTION

The invention provides for methods for identifying the basal or luminal phenotype of a cell, comprising: (a) measuring the expression level of one gene selected from the group consisting of the genes encoding SCNN1A, CA12, PRKX, TFF3, HNF3A, MYB, GABRP, ESR1, AGR2, GATA3, FOXC1, and EN1 in a patient sample; and (b) comparing the expression level of the gene from a sample with the expression level of the gene in a normal tissue sample or a reference expression level, wherein an increase of expression in the patient sample of one gene selected from the group consisting of the genes encoding PRKX, GABRP, FOXC1, and EN1 indicates the cell has a basal phenotype and an increase of expression of one gene selected from the group consisting of the genes encoding SCNN1A, CA12, TFF3, HNF3A, MYB, ESR1, AGR2, and GATA3 indicates the cell has a luminal phenotype.

Thus, in some embodiments, a method for identifying basal-positive cancer patient, comprising: (a) measuring the expression level of one gene selected from the group consisting of the genes encoding SCNN1A, CA12, PRKX, TFF3, HNF3A, MYB, GABRP, ESR1, AGR2, GATA3, FOXC1, and EN1 in a sample from the patient; and (b) comparing the expression level of the gene from the patient with the expression level of the gene in a normal tissue sample or a reference expression level, wherein an increase of expression of one gene selected from the group consisting of the genes encoding PRKX, GABRP, FOXC1, and EN1 indicates the patient has basal type cancer and an increase of expression of one gene selected from the group consisting of the genes encoding SCNN1A, CA12, TFF3, HNF3A, MYB, ESR1, AGR2, and GATA3 indicates the patient does not have basal-positive cancer.

The invention provides for a method for identifying a cancer patient suitable for treatment with a conformationally-restricted polyamine, CGC-11047, comprising detecting modulated expression of genes selected from the group consisting of: RPL15, RAD54B, NEB, STAG2, MTAP, WASL, GCLM, CST3, LAMA3, SSRP1, ACYP1, CYLD, PRPF18, AMFR, DEAF1, PPP1R2, LOH11CR2A, and ACSL3.

The invention provides for a method for identifying a cancer patient suitable for treatment with a conformationally-restricted polyamine, CGC-11047, wherein the patient (a) is basal-like-positive and (b) has an increased or high expression level of RAD54B, STAG2, MTAP GCLM, LAMA3, SSRP1, ACYP1, CYLD, PRPF18, AMFR, PPP1R2, or LOH11CR2A. Patients identified as having an increased expression of these genes are predicted to be sensitive to treatment of cancer with a conformationally-restricted polyamine, such as CGC-11047. In other embodiments, if the patient is determined to have an increased or high expression level of one or more of the genes encoding RPL15, NEBL, WASL, CST3, DEAF1, or ACSL3 are predicted to be resistant to treatment of cancer with a conformationally-restricted polyamine.

In some embodiments of the invention, an increased or decreased expression level is an expression level of a gene that is more than or less than, respectively, than the expression level of the same gene in a normal tissue or cell sample, such as the cell or tissue sample of non-cancerous cells of the patient or another person that does not have cancer.

In some embodiments of the invention, an increased or decreased expression level is an expression level of a gene that is more than or less than, respectively, than the average expression level of the same gene in a panel of normal cell lines or cancer cell lines

The invention provides for a method of treating a cancer patient comprising (a) identifying a cancer patient who is suitable for treatment with a conformationally-restricted polyamine using a method of the present invention, and (b) administering a therapeutically effective amount of the conformationally-restricted polyamine to the patient.

The invention also provides a computational model useful for identifying a cancer patient suitable for treatment with a conformationally-restricted polyamine, such as CGC-11047.

In some embodiments, the cancer is breast cancer and the cancer patient is a breast cancer patient. In certain embodiments, the breast cancer patient is a basal cell-positive breast cancer patient.

### BRIEF DESCRIPTION OF THE DRAWINGS

FIG. 1. A histogram depicting the relative sensitivity of the amount of compound CGC-11047 (also known as SL-11047) required to inhibit the growth of a host of different breast cancers by 50% (the GI₅₀). Linear relative sensitivity is depicted on the Y-axis; different breast cancer cell lines are depicted on the X-axis.

FIG. 2. Results from a network association analyses with Ingenuity, showing that 11 genes that predict sensitivity to CGC-11047 are inter-related, either directly or indirectly through other genes, in networks involved with actin or integrin.

FIG. 3. Effect of CGC-11047 on various cancer cell lines at concentrations between 1×10⁻⁸ and 1×10⁻² M. CGC-11047 is depicted on the X-axis, and percent growth is depicted on the Y-axis.

FIGS. 4A and 4B. Effect of CGC-11047 on HCC70 cells with respect to cell cycle and apoptosis. In FIG. 4A, cells were treated for 72 hours with 0, 0.3, 10, or 100 μM CGC-11047. Shown are percentage of the cell population is G0-G1, S, or G2-M phase of cell growth. In FIG. 4B, cells were treated for 42, 48, or 72 hours with 0.3, 10, or 300 μM CGC-11047. Shown is the caspase activity (apoptosis) of treated cells within each condition compared to untreated controls.

FIGS. 5A and 5B. Effect of CGC-11047 on T47D cells with respect to cell cycle and apoptosis. In FIG. 5A, cells were treated for 72 hours with 0, 0.3, 10, or 100 μM CGC-11047. Shown are percentage of the cell population is G0-G1, S, or G2-M phase of cell growth. In FIG. 5B, cells were treated for 42, 48, or 72 hours with 0.3, 10, or 300 μM CGC-11047. Shown is the caspase activity (apoptosis) of treated cells within each condition compared to untreated controls.

### DETAILED DESCRIPTION OF THE INVENTION

Before the present invention is described, it is to be understood that this invention is not limited to particular embodiments described, and as such may vary. It is also to be understood that the terminology used herein is for the purpose of describing particular embodiments only, and is not intended to be limiting; the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a gene” includes a plurality of such genes, and so forth.

Various embodiments of the invention are more fully described as follows.

### Basal or Luminal Cancer Phenotype Markers

A comprehensive analysis of gene expression analysis was applied in 130 primary breast tumors from the University of California San Francisco training set and 48 breast cancer cell lines. The expression levels of the following genes were found to predict either a basal or luminal phenotype of a cell: SCNN1A, CA12, PRKX, TFF3, HNF3A, MYB, GABRP, ESR1, AGR2, GATA3, FOXC1, and EN1. High expression of PRKX, GABRP, FOXC1, or EN1 and/or low expression of SCNN1A, CA12, TFF3, HNF3A, MYB, ESR1, AGR2, or GATA3 predicted a basal phenotype. Conversely, low expression of PRKX, GABRP, FOXC1, or EN1 and/or high expression of SCNN1A, CA12, TFF3, HNF3A, MYB, ESR1, AGR2, or GATA3 predicted a luminal phenotype.

Thus, one embodiment provides for a method for identifying the basal or luminal phenotype of a cell, comprising: (a) measuring the expression level of one gene selected from the group consisting of the genes encoding SCNN1A, CA12, PRKX, TFF3, HNF3A, MYB, GABRP, ESR1, AGR2, GATA3, FOXC1, and EN1, in a patient sample; and (b) comparing the expression level of the gene from a sample with the expression level of the gene in a normal tissue sample or a reference expression level, wherein an increase of expression in the patient sample of one gene selected from the group consisting of the genes encoding PRKX, GABRP, FOXC1, and EN1, indicates the cell has a basal phenotype and an increase of expression of one gene selected from the group consisting of the genes encoding SCNN1A, CA12, TFF3, HNF3A, MYB, ESR1, AGR2, and GATA3, indicates the cell has a luminal phenotype.

In another embodiment, a decrease of expression in the patient sample of one gene selected from the group consisting of the genes encoding PRKX, GABRP, FOXC1, and EN1, indicates the cell has a luminal phenotype, and a decrease of expression of one gene selected from the group consisting of the genes encoding SCNN1A, CA12, TFF3, HNF3A, MYB, ESR1, AGR2, and GATA3, indicates the cell has a basal phenotype.

Detection of a basal-like cell phenotype further indicates the presence of aggressive cancers, i.e., the presence of clinically aggressive basal A subtype cancer cells in the tissue are likely to increase tumor progression and metastasize to other tissues. Thus, another embodiment provides for a prognostic method for predicting the outcome of a patient by detection of modulated expression of one of the basal/luminal gene markers in a patient tissue or biopsy using immunohistochemistry (IHC) as compared to normal levels in a control sample. Overexpression of PRKX, GABRP, FOXC1, and EN1 and/or a decrease in expression levels of SCNN1A, CA12, TFF3, HNF3A, MYB, ESR1, AGR2, and GATA3 as detected can be used as an indicator of basal cancer, and thus indicate that metastatic or invasive cells are present in the patient tissue, which may likely lead to metastatic cancer in the near future. In another embodiment, overexpression of PRKX, GABRP, FOXC1, and EN1 and/or a decrease in expression levels of SCNN1A, CA12, TFF3, HNF3A, MYB, ESR1, AGR2, and GATA3 can be determined by comparison to a reference expression level (such as the average expression level of the gene in a cell line panel or a cancer cell or tumor panel, or the like).

Another embodiment provides for a prognostic method to provide more accurate prognosis for patients having non-invasive cancer (e.g., lymph-node negative cancer) previously determined based on morphology by a pathologist. A new biopsy can be taken or biopsies previously taken and preserved (e.g., in paraffin) can be used. In addition to observing morphology of a tumor (e.g., histological grade, stage and size), detection of modulated expression of any of the 12 basal/luminal markers can be carried out by IHC assay and a new prognosis determined, factoring in the finding of level of modulated expression levels. For example, a finding by IHC that basal cell markers are present at an increased level as compared to a normal tissue, despite the morphology of a non-invasive cancer, will indicate that the tumor should be staged or graded higher as a tumor that will be invasive and aggressive, leading to metastasis.

The basal/luminal gene markers described herein are not limited to breast cancer. They can be used to determine the basal or luminal phenotype, diagnose the metastatic and aggressive nature, or determine the prognosis of any cancer. Such cancers include but are not limited to those occurring in the ovary, bladder, head, and neck, and further include epithelial, cervical, endometrial, lung, and prostate cancers.

### Sensitivity of Basal Cancer Phenotype to Conformationally-Restricted Polyamines

The present inventors undertook a study of the inhibition of cell growth caused by exposure to various conformationally-constrained polyamine-type chemotherapeutic agents. Polyamine-type chemotherapeutic agents are defined herein as any chemotherapeutic agent that is a spermine or spermidine derivative or analog and, preferably, has a cyclic or double-bond constraint in the backbone.

As described herein, conformationally-constrained polyamines include, without limitation, compounds of Formula I:

E-NH-D-NH-B-A-B-NH-D-NH-E (Formula I)
wherein A is selected from the group consisting of C₂-C₆ alkene and C₃-C₆ cycloalkyl, cycloalkenyl, and cycloaryl;
B is independently selected from the group consisting of a single bond and C₁-C₆ alkyl and alkenyl;
D is independently selected from the group consisting of C₁-C₆ alkyl and alkenyl, and C₃-C₆ cycloalkyl, cycloalkenyl, and cycloaryl;
E is independently selected from the group consisting of H, Cᵢ-C₆ alkyl and alkenyl;
   and pharmaceutically-suitable salts thereof.

An illustrative listing of compounds of Formula I is presented in Table 1.

TABLE 1

| Compound | | |
|---|---|---|
| No. | | |
| 12 | 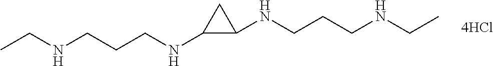 | SL-11027 |
| 13 | 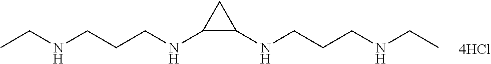 | SL-11033 |
| 23 | 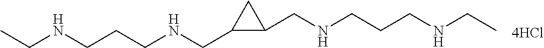 | SL-11038 |
| 28 | 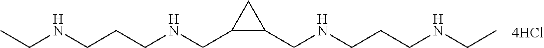 | SL-11037 |
| 35 | 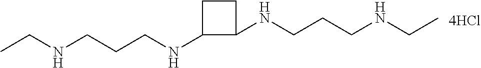 | SL-11028 |
| 36 | 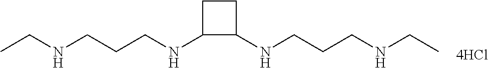 | SL-11034 |
| 47 | 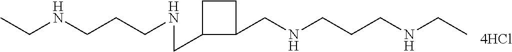 | SL-11044 |
| 48 | 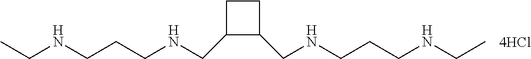 | SL-11043 |
| 57 | 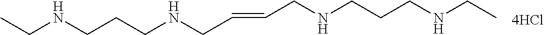 | SL-11048 |
| 58 | 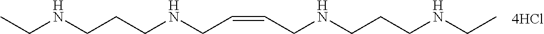 | SL-11047 |

As described herein, analysis of cellular and molecular responses to CGC-11047 (SL-11047 or Compound 58 in Table 1) were performed. CGC-11047 was added to a panel of 48 breast cell lines (including 42 breast cancer cell lines) at nine different serial dilutions between 5×10⁻³ and 1.3×10⁻⁸ M. The GI₅₀ (inhibition of growth by 50%) was calculated. These analyses demonstrated that CGC-11047 is more cytotoxic in cell lines representing clinically aggressive basal A breast cancers subtype than in luminal subtype cell lines or non-transformed human mammary epithelial cultures. Thus, the markers that differentiate basal versus luminal cancer subtypes (e.g., SCNN1A, CA12, PRKX, TFF3, HNF3A, MYB, GABRP, ESR1, AGR2, GATA3, FOXC1, and EN1) can serve as predictive markers of sensitivity or resistance to conformationally-restricted polyamines such as CGC-11047.

### Predictors of Sensitivity to Conformationally-Restricted Polyamines

Other predictive biomarkers of sensitivity to CGC-11047 treatment were determined by arraying genes that change expression in response to CGC-11047. These biomarkers were discovered by supervising genomic and gene expression signatures of cell lines with the GI₅₀ profile of CGC-11047. One group of predictive markers includes the following genes: RPL15, RAD54B, NEB, STAG2, and MTAP. It was found with this group that an increase in gene expression levels of RAD54B, STAG2, or MTAP and/or a decrease in expression levels of RPL15 or NEBL indicate a sensitivity of the cell to CGC-11047. By contrast, the inverse, wherein a decrease in gene expression levels of RAD54B, STAG2, or MTAP and/or an increase in expression levels of RPL15 or NEBL indicates a resistance of the cell to CGC-11047.

Thus, another embodiment provides for a method for identifying the sensitivity or resistance of a cancer cell to conformationally-restricted polyamine treatment, comprising: (a) measuring the expression level of a gene selected from the group consisting of the genes encoding RPL15, RAD54B, NEB, STAG2, and MTAP in a patient sample; and (b) comparing the expression level of the gene from a sample with the expression level of the gene in a normal tissue sample or a reference expression level, wherein an increase in expression of RAD54B, STAG2, and MTAP and/or a decrease in expression levels of RPL15 and NEBL indicate a sensitivity of the cell to conformationally-restricted polyamines, and wherein a decrease in gene expression levels of RAD54B, STAG2, and MTAP and/or an increase in expression levels of RPL15 and NEBL indicates a resistance of the cell to conformationally-restricted polyamines.

A second group of predictive markers includes the following genes: WASL, GCLM, CST3, LAMA3, SSRP1, ACYP1, CYLD, PRPF18, AMFR, DEAF1, PPP1R2, LOH11CR2A, and ACSL3. It was found with this group that that an increase in gene expression levels of GCLM, LAMA3, SSRP1, ACYP1, CYLD, PRPF18, AMFR, PPP1R2, or LOH11CR2A and/or a decrease in expression levels of WASL, CST3, DEAF1, or ACSL3 indicate a sensitivity of the cell to CGC-11047. By contrast, the inverse, wherein a decrease in gene expression levels of GCLM, LAMA3, SSRP1, ACYP1, CYLD, PRPF18, AMFR, PPP1R2, or LOH11CR2A and/or an increase in expression levels of WASL, CST3, DEAF1 or ACSL3 indicates a resistance of the cell to CGC-11047.

Thus, another embodiment provides for a method for identifying the sensitivity or resistance of a cancer cell to conformationally-restricted polyamine treatment, comprising: (a) measuring the expression level of a gene selected from the group consisting of the genes encoding WASL, GCLM, CST3, LAMA3, SSRP1, ACYP1, CYLD, PRPF18, AMFR, DEAF1, PPP1R2, LOH11CR2A, and ACSL3 in a patient sample; and (b) comparing the expression level of the gene from a sample with the expression level of the gene in a normal tissue sample or a reference expression level, wherein an increase in gene expression levels of GCLM, LAMA3, SSRP1, ACYP1, CYLD, PRPF18, AMFR, PPP1R2, and LOH11CR2A and/or a decrease in expression levels of WASL, CST3, DEAF1, and ACSL3 indicate a sensitivity of the cell to conformationally-restricted polyamines, and wherein a decrease in gene expression levels of GCLM, LAMA3, SSRP1, ACYP1, CYLD, PRPF18, AMFR, PPP1R2, and LOH11CR2A and/or an increase in expression levels of WASL, CST3, DEAF1, and ACSL3 indicates a resistance of the cell to conformationally-restricted polyamines.

Combining the basal/luminal cell markers with the other predictive markers of sensitivity to conformational-restricted polyamines, yet another embodiment provides for a method for identifying the sensitivity or resistance of a cancer cell to conformationally-restricted polyamine treatment, comprising: (a) measuring the expression level of a gene selected from the group consisting of the genes encoding SCNN1A, CA12, TFF3, HNF3A, MYB, ESR1, AGR2, GATA3, RPL15, RAD54B, NEB, STAG2, MTAP, WASL, GCLM, CST3, LAMA3, SSRP1, ACYP1, CYLD, PRPF18, AMFR, DEAF1, PPP1R2, LOH11CR2A, and ACSL3 in a patient sample; and (b) comparing the expression level of the gene from a sample with the expression level of the gene in a normal tissue sample or a reference expression level, wherein an increase in gene expression levels of PRKX, GABRP, FOXC1, EN1, RAD54B, STAG2, and MTAP, GCLM, LAMA3, SSRP1, ACYP1, CYLD, PRPF18, AMFR, PPP1R2, and LOH11CR2A and/or a decrease in expression levels of SCNN1A, CA12, TFF3, HNF3A, MYB, ESR1, AGR2, GATA3, RPL15, NEBL WASL, CST3, DEAF1, and ACSL3 indicate a sensitivity of the cell to conformationally-restricted polyamines, and wherein a decrease in gene expression levels of PRKX, GABRP, FOXC1, EN1, RAD54B, STAG2, and MTAP, GCLM, LAMA3, SSRP1, ACYP1, CYLD, PRPF18, AMFR, PPP1R2, and LOH11CR2A and/or an increase in expression levels of SCNN1A, CA12, TFF3, HNF3A, MYB, ESR1, AGR2, GATA3, RPL15, NEBL WASL, CST3, DEAF1, and ACSL3 indicates a resistance of the cell to conformationally-restricted polyamines.

In another embodiment, an increase in the expression level of one or more of RAD54B, STAG2, MTAP, GCLM, LAMA3, SSRP1, ACYP1, CYLD, PRPF18, AMFR, PPP1R2, and LOH11CR2A in a patient sample, as compared to the expression level of each corresponding gene in a normal tissue sample or a reference expression level (such as the average expression level of the gene in a cell line panel or a cancer cell or tumor panel, or the like), indicates that the cancer cell, tissue or tumor, from which the patient sample was obtained, is sensitive to treatment with conformationally-restricted polyamines.

In anther embodiment, a decrease in the expression level of one or more of RPL15, NEBL, WASL, CST3, DEAF 1, and ACSL3 in a patient sample, as compared to the expression level of each corresponding gene in a normal tissue sample or a reference expression level (such as the average expression level of the gene in a cell line panel or a cancer cell or tumor panel, or the like), indicates that the cancer cell, tissue or tumor, from which the patient sample was obtained, is sensitive to treatment with conformationally-restricted polyamines

In some embodiments, a decrease in the expression levels of any two, three or four of RPL15, NEBL, WASL, CST3, DEAF1, and ACSL3 in a patient sample, as compared to the expression level of each gene in a normal tissue sample or a reference expression level (such as the average expression level of the gene in a cell line panel or a cancer cell or tumor panel, or the like), indicates that the cancer cell, tissue or tumor, from which the patient sample was obtained, is sensitive to treatment with conformationally-restricted polyamines

In some embodiments of the invention, the method comprises: (a) measuring the expression level of one gene selected from the group consisting of the genes encoding RPL15, RAD54B, NEB, STAG2, MTAP, WASL, GCLM, CST3, LAMA3, SSRP1, ACYP1, CYLD, PRPF18, AMFR, DEAF1, PPP1R2, LOH11CR2A, and ACSL3 in a sample from the patient; and (b) determining the response of the breast cancer to a conformationally-restricted polyamine. The level and type of response can be determined through various statistical analyses as demonstrated in the Examples and including but not limited to, association and cluster analysis, Kaplan-Meier hierarchical cluster analysis, multivariate analysis, principle component analysis and Pearson correlations and other computation models.

In some embodiments of the invention, the method further comprises administering a therapeutically effective amount of the conformationally-restricted polyamine to the patient. Compounds and formulations of conformationally-restricted polyamines suitable for use in the present invention are taught in U.S. Pat. No. 5,899,061. Dosages and methods of administration of conformationally-restricted polyamines are taught in U.S. Pat. No. 7,186,825.

The predictors described above (e.g., RPL15, RAD54B, NEB, STAG2, MTAP, WASL, GCLM, CST3, LAMA3, SSRP1, ACYP1, CYLD, PRPF18, AMFR, DEAF1, PPP1R2, LOH11CR2A, and ACSL3) are not limited to breast cancer cells. They can be used to predict the sensitivity or resistance of a cancer cell to conformationally-restricted polyamines for any type of cancer. Such cancers include but are not limited to those occurring in the ovary, bladder, head, and neck, and further include epithelial, cervical, endometrial, lung, and prostate cancers.

The described predictors could be used to enrich patient populations for potential responders prior to initiating therapy in the clinic and to define tailored therapeutics for individual patients.

### Implementing Basal/Luminal Markers and Conformationally-Restricted Polyamine Response Indicators

Individual breast cancers vary in the way they respond to molecularly targeted therapies because they vary in the spectrum of genomic, biological and epigenomic abnormalities accumulated during progression to the malignant state. The panel of 48 breast cancer cell lines has been previously found to mirror the recurrent abnormalities found in primary tumors as well as the variability therein. Therefore, molecular predictors of response to targeted therapies in patients should be the same as those that predict change in growth rate, apoptosis and/or change in cell cycle distribution in cell lines grown in vitro. Thus, the markers of a basal and luminal cancer cell phenotype and the poor and sensitive response indicators to CGC-11047 found in the panel of 48 breast cell lines can be used as molecular predictors of response to targeted therapies and identifying patients predicted to have poor and sensitive response to CGC-11047.

The present Examples and measured responses to CGC-11047 are also contemplated to be applicable to other similar compounds that target basal-like cancers. In one embodiment, the predictive gene markers described herein are predictive of sensitivity to other conformationally-restricted polyamines including those listed in Table 1 and as described in U.S. Pat. No. 5,889,061, hereby incorporated by reference. Thus, in any of the embodiments described herein, CGC-11047 may be substituted with any other conformationally-restricted polyamine described herein and in U.S. Pat. No. 5,889,061.

In any of the embodiments described herein, step (a) may comprise measuring more than one gene selected from the group consisting of the genes encoding SCNN1A, CA12, TFF3, HNF3A, MYB, ESR1, AGR2, GATA3, RPL15, RAD54B, NEB, STAG2, MTAP, WASL, GCLM, CST3, LAMA3, SSRP1, ACYP1, CYLD, PRPF18, AMFR, DEAF1, PPP1R2, LOH11CR2A, and ACSL3 in a sample from the patient. “More than one gene” may include at least two genes, at least three genes, at least four genes, and so on.

The expression level of a gene is measured from a sample from the patient that comprises essentially a cancer cell or cancer tissue of a cancer tumor. Methods for obtaining such samples are well known to those skilled in the art. When the cancer is breast cancer, the expression level of a gene is measured from a sample from the patient that comprises essentially a breast cancer cell or breast cancer tissue of a breast cancer tumor.

The expression levels may be measured in a basal state, i.e., untreated with a conformationally-restricted polyamine, as would occur for distinguishing between basal and luminal subtypes, or after treatment with the compounds, as would occur to detect the other predictive markers described herein. In the latter case, the compound may either be administered to the patient prior to obtaining cellular samples or added directly to the cells after obtaining them.

The expression level of a gene may be measured by any means now known or developed in the future. For example, the expression level of a gene may be measured by measuring the amount or number of molecules of mRNA or transcript in a cell. The measuring can comprise directly measuring the mRNA or transcript obtained from a cell, or measuring the cDNA obtained from an mRNA preparation thereof. Such methods of extracting the mRNA or transcript from a cell, or preparing the cDNA thereof are well known to those skilled in the art.

Gene expression of the markers described herein can also be analyzed by techniques known in the art, e.g., reverse transcription and amplification of mRNA, isolation of total RNA or poly A+RNA, northern blotting, dot blotting, in situ hybridization, RNase protection, probing DNA microchip arrays, and the like. These techniques can be performed with knowledge of the polynucleotide sequence of the target gene or mRNA. The mRNA of each gene marker identified herein is described below.

In other embodiments, the expression level of a gene can be measured by measuring or detecting the amount of protein or polypeptide expressed, such as measuring the amount of antibody that specifically binds to the protein in a dot blot or Western blot. The proteins described in the present invention can be overexpressed and purified or isolated to homogeneity and antibodies raised that specifically bind to each protein. Such methods are well known to those skilled in the art and are described in more detail below.

Other methods of measuring gene expression, or genome copy number abnormalities that may affect gene expression, may include any of the following: immunohistochemistry (IHC), methods that utilize fluorescence in situ hybridization (FISH), comparative genomic hybridization (CGH), single-nucleotide polymorphism (SNP) arrays. A commercially available IHC test is PathVysion® (Vysis Inc., Downers Grove, Ill.). A commercially available FISH test is DAKO HercepTest® (DAKO Corp., Carpinteria, Calif.). Commercially available arrays include Affymetrix 250K SNP arrays (20 Kbp resolution) (Affymetrix, Santa Clara, Calif.) and Affymetrix Molecular Inversion Probe allele-specific CGH (single gene resolution). There are several publicly-accessible, fee-for-services laboratories that will perform CGH analyses, such as the Fred Hutchinson Cancer Research Center, Seattle, Wash. Gene expression patterns can also be measured using Affymetrix U133A arrays, which are described in detail in the Examples. Other commercial kits for measuring gene transcription include assays from Luminex Corporation (Austin, Tex.) (e.g., Lumiunex's “xMAP”-brand protocol) and Panomics, Inc. (Fremont, Calif.). Methods of detecting gene expression or genome copy number such as FISH and IHC with a given nucleotide sequence are described in detail in PCT/US2006/002202.

The expression level of a gene encoding SCNN1A, CA12, PRKX, TFF3, HNF3A, MYB, GABRP, ESR1, AGR2, GATA3, FOXC1, EN1, RPL15, RAD54B, NEB, STAG2, MTAP, WASL, GCLM, CST3, LAMA3, SSRP1, ACYP1, CYLD, PRPF18, AMFR, DEAF1, PPP1R2, LOH11CR2A, and ACSL3 can be measured using an oligonucleotide derived from their mRNA nucleotide sequences using one of the above-described methods. For a listing of the genes with their nucleotide and amino acid sequence list identifiers, see Table 2.

TABLE 2

| Gene Name | Nucleotide Sequence | Amino Acid Sequence |
|---|---|---|
| WASL | SEQ. ID. NO: 1 | SEQ. ID. NO: 2 |
| GCLM | SEQ. ID. NO: 3 | SEQ. ID. NO: 4 |
| CST3 | SEQ. ID. NO: 5 | SEQ. ID. NO: 6 |
| LAMA3 | SEQ. ID. NO: 7 | SEQ. ID. NO: 8 |
| SSRP1 | SEQ. ID. NO: 9 | SEQ. ID. NO: 10 |
| ACYP1 | SEQ. ID. NO: 11 | SEQ. ID. NO: 12 |
| CYLD | SEQ. ID. NO: 13 | SEQ. ID. NO: 14 |
| PRPF18 | SEQ. ID. NO: 15 | SEQ. ID. NO: 16 |
| AMFR | SEQ. ID. NO: 17 | SEQ. ID. NO: 18 |
| DEAF1 | SEQ. ID. NO: 19 | SEQ. ID. NO: 20 |
| PPP1R2 | SEQ. ID. NO: 21 | SEQ. ID. NO: 22 |
| LOH11CR2A | SEQ. ID. NO: 23 | SEQ. ID. NO: 24 |
| ACSL3 | SEQ. ID. NO: 25 | SEQ. ID. NO: 26 |
| SCNN1A | SEQ. ID. NO: 27 | SEQ. ID. NO: 28 |
| CA12 | SEQ. ID. NO: 29 | SEQ. ID. NO: 30 |
| PRKX | SEQ. ID. NO: 31 | SEQ. ID. NO: 32 |
| TFF3 | SEQ. ID. NO: 33 | SEQ. ID. NO: 34 |
| HNF3A (FOXA1) | SEQ. ID. NO: 35 | SEQ. ID. NO: 36 |
| MYB, 4602 | SEQ. ID. NO: 37 | SEQ. ID. NO: 38 |
| GABRP | SEQ. ID. NO: 39 | SEQ. ID. NO: 40 |
| ESR1 | SEQ. ID. NO: 41 | SEQ. ID. NO: 42 |
| AGR2 | SEQ. ID. NO: 43 | SEQ. ID. NO: 44 |
| GATA3 | SEQ. ID. NO: 45 | SEQ. ID. NO: 46 |
| FOXC1 | SEQ. ID. NO: 47 | SEQ. ID. NO: 48 |
| EN1 | SEQ. ID. NO: 49 | SEQ. ID. NO: 50 |
| RPL15 | SEQ. ID. NO: 51 | SEQ. ID. NO: 52 |
| RAD54B | SEQ. ID. NO: 53 | SEQ. ID. NO: 54 |
| NEBL | SEQ. ID. NO: 55 | SEQ. ID. NO: 56 |
| STAG2 | SEQ. ID. NO: 57 | SEQ. ID. NO: 58 |
| MTAP | SEQ. ID. NO: 59 | SEQ. ID. NO: 60 |

The protein expressed by the WASL gene (SEQ ID NO:2) is also known as Wiskott-Aldrich syndrome-like (WASL). The expression level of a gene encoding WASL can be measured using an oligonucleotide derived from SEQ ID NO:1 (GenBank Accession No. BE504979), the mRNA transcript encoding the WASL protein.

The protein expressed by the GCLM gene (SEQ ID NO:4) is also known as glutamate-cysteine ligase, modifier subunit (GCLM). The expression level of a gene encoding GCLM can be measured using an oligonucleotide derived from SEQ ID NO:3 (GenBank Accession No. NM_{—}002061), the mRNA transcript encoding the GCLM protein.

The protein expressed by the CST3 gene (SEQ ID NO:6) is also known as cystatin C (amyloid angiopathy and cerebral hemorrhage) (CST3). The expression level of a gene encoding CST3 can be measured using an oligonucleotide derived from SEQ ID NO:5 (GenBank Accession No. NM_{—}000099), the mRNA transcript encoding the CST3 protein.

The protein expressed by the LAMA3 gene (SEQ ID NO:8) is also known as laminin, alpha 3 (LAMA3). The expression level of a gene encoding LAMA3 can be measured using an oligonucleotide derived from SEQ ID NO:7 (GenBank Accession No. NM_{—}000227), the mRNA transcript encoding the LAMA3 protein.

The protein expressed by the SSRP1 gene (SEQ ID NO:10) is also known as structure specific recognition protein 1 (SSRP1). The expression level of a gene encoding SSRP1 can be measured using an oligonucleotide derived from SEQ ID NO:9 (GenBank Accession No. BE795648), the mRNA transcript encoding the SSRP1 protein.

The protein expressed by the ACYP1 gene (SEQ ID NO:12) is also known as acylphosphatase 1, erythrocyte (common) type (ACYP1). The expression level of a gene encoding ACYP1 can be measured using an oligonucleotide derived from SEQ ID NO:11 (GenBank Accession No. NM_{—}001107), the mRNA transcript encoding the ACYP1 protein.

The protein expressed by the CYLD gene (SEQ ID NO:14) is also known as cylindromatosis (turban tumor syndrome) (CYLD). The expression level of a gene encoding CYLD can be measured using an oligonucleotide derived from SEQ ID NO:13 (GenBank Accession No. BE046443), the mRNA transcript encoding the CYLD protein.

The protein expressed by the PRPF18 gene (SEQ ID NO:16) is also known as PRP18 pre-mRNA processing factor 18 homolog (S. cerevisiae) (PRPF18). The expression level of a gene encoding PRPF18 can be measured using an oligonucleotide derived from SEQ ID NO:15 (GenBank Accession No. BC000794), the mRNA transcript encoding the PRPF18 protein.

The protein expressed by the AMFR gene (SEQ ID NO:18) is also known as autocrine motility factor receptor (AMFR). The expression level of a gene encoding AMFR can be measured using an oligonucleotide derived from SEQ ID NO:17 (GenBank Accession No. NM_{—}001144), the mRNA transcript encoding the AMFR protein.

The protein expressed by the DEAF1 gene (SEQ ID NO:20) is also known as deformed epidermal autoregulatory factor 1 (Drosophila) (DEAF1). The expression level of a gene encoding DEAF1 can be measured using an oligonucleotide derived from SEQ ID NO:19 (GenBank Accession No. AF068892), the mRNA transcript encoding the DEAF 1 protein.

The protein expressed by the PPP1R2 gene (SEQ ID NO:22) is also known as protein phosphatase 1, regulatory (inhibitor) subunit 2 (PPP1R2). The expression level of a gene encoding PPP1R2 can be measured using an oligonucleotide derived from SEQ ID NO:21 (GenBank Accession No. NM_{—}006241), the mRNA transcript encoding the PPP1R2 protein.

The protein expressed by the LOH11CR2A gene (SEQ ID NO:24) is also known as loss of heterozygosity, 11, chromosomal region 2, gene A (LOH11CR2A). The expression level of a gene encoding LOH11CR2A can be measured using an oligonucleotide derived from SEQ ID NO:23 (GenBank Accession No. BC001234), the mRNA transcript encoding the LOH11CR2A protein.

The protein expressed by the ACSL3 gene (SEQ ID NO:26) is also known as acyl-CoA synthetase long-chain family member 3 (ACSL3). The expression level of a gene encoding ACSL3 can be measured using an oligonucleotide derived from SEQ ID NO:25 (GenBank Accession No. NM_{—}004457), the mRNA transcript encoding the ACSL3 protein.

The protein expressed by the SCNN1A gene (SEQ ID NO:28) is also known as sodium channel, nonvoltage-gated 1 alpha (SCNN1A). The expression level of a gene encoding SCNN1A can be measured using an oligonucleotide derived from SEQ ID NO:27 (GenBank Accession No. NM_{—}001038), the mRNA transcript encoding the SCNN1A protein.

The protein expressed by the CA12 gene (SEQ ID NO:30) is also known as carbonic anhydrase XII (CA12). The expression level of a gene encoding CA12 can be measured using an oligonucleotide derived from SEQ ID NO:29 (GenBank Accession No. NM_{—}001218), the mRNA transcript encoding the CA12 protein.

The protein expressed by the PRKX gene (SEQ ID NO:32) is also known as PKX1 or protein kinase, X-linked. The expression level of a gene encoding PRKX can be measured using an oligonucleotide derived from SEQ ID NO:31 (GenBank Accession No. NM_{—}005044), the mRNA transcript encoding the PRKX protein.

The protein expressed by the TFF3 gene (SEQ ID NO:34) is also known as trefoil factor 3 (TFF3). The expression level of a gene encoding TFF3 can be measured using an oligonucleotide derived from SEQ ID NO:33 (GenBank Accession No. NM_{—}003226), the mRNA transcript encoding the TFF3 protein.

The protein expressed by the HNF3A gene (SEQ ID NO:36) is also known as forkhead box A1 (FOXA1) or hepatocyte nuclear factor 3 alpha (HNF3A). The expression level of a gene encoding HNF3A can be measured using an oligonucleotide derived from SEQ ID NO:35 (GenBank Accession No. NM_{—}004496), the mRNA transcript encoding the HNF3A protein.

The protein expressed by the MYB gene (SEQ ID NO:38) is also known as v-myb myeloblastosis viral oncogene homolog (avian) (MYB). The expression level of a gene encoding MYB can be measured using an oligonucleotide derived from SEQ ID NO:37 (GenBank Accession No. NM_{—}005375), the mRNA transcript encoding the MYB protein.

The protein expressed by the GABRP gene (SEQ ID NO:40) is also known as gamma-aminobutyric acid (GABA) A receptor, pi (GABRP). The expression level of a gene encoding GABRP can be measured using an oligonucleotide derived from SEQ ID NO:39 (GenBank Accession No. NM_{—}014211), the mRNA transcript encoding the GABRP protein.

The protein expressed by the ESR1 gene (SEQ ID NO:42) is also known estrogen receptor 1 (ESR1). The expression level of a gene encoding ESR1 can be measured using an oligonucleotide derived from SEQ ID NO:41 (GenBank Accession No. NM_{—}000125), the mRNA transcript encoding the ESR1 protein.

The protein expressed by the AGR2 gene (SEQ ID NO:44) is also known as anterior gradient 2 homolog (Xenepus laevis) (AGR2). The expression level of a gene encoding AGR2 can be measured using an oligonucleotide derived from SEQ ID NO:43 (GenBank Accession No. NM_{—}006408), the mRNA transcript encoding the AGR2 protein.

The protein expressed by the GATA3 gene (SEQ ID NO:46) is also known as GATA binding protein 3 (GATA3). The expression level of a gene encoding GATA3 can be measured using an oligonucleotide derived from SEQ ID NO:45 (GenBank Accession Nos. NM_{—}002051 and NM_{—}032742), the mRNA transcripts encoding the GATA3 protein.

The protein expressed by the FOXC1 gene (SEQ ID NO:48) is also known as Homo sapiens cDNA FLJ11796 fis, clone HEMBA1006158, highly similar to Homo sapiens transcription factor forkhead-like 7 (FKHL7) gene. The expression level of a gene encoding FOXC1 can be measured using an oligonucleotide derived from SEQ ID NO:47 (GenBank Accession No. AK021858), the mRNA transcript encoding the FOXC1 protein.

The protein expressed by the EN1 gene (SEQ ID NO:50) is also known as engrailed homolog 1 (EN1). The expression level of a gene encoding EN1 can be measured using an oligonucleotide derived from SEQ ID NO:49 (GenBank Accession No. NM_{—}001426), the mRNA transcript encoding the EN1 protein.

The protein expressed by the RPL15 gene (SEQ ID NO:52) is also known as ribosomal protein L15 (RPL15). The expression level of a gene encoding RPL15 can be measured using an oligonucleotide derived from SEQ ID NO:51 (GenBank Accession No. NM_{—}002948), the mRNA transcript encoding the RPL15 protein.

The protein expressed by the RAD54B gene (SEQ ID NO:54) is also known as RAD54B homolog (RAD54B). The expression level of a gene encoding RAD54B can be measured using an oligonucleotide derived from SEQ ID NO:53 (GenBank Accession Nos. NM_{—}012415 and NM_{—}134434), the mRNA transcript encoding the RAD54B protein.

The protein expressed by the NEBL gene (SEQ ID NO:56) is also known as nebulette (NEBL). The expression level of a gene encoding NEBL can be measured using an oligonucleotide derived from SEQ ID NO:55 (GenBank Accession No. NM_{—}006393), the mRNA transcript encoding the NEBL protein.

The protein expressed by the STAG2 gene (SEQ ID NO:58) is also known as stromal antigen 2 (STAG2). The expression level of a gene encoding STAG2 can be measured using an oligonucleotide derived from SEQ ID NO:57 (GenBank Accession No. NM_{—}006603), the mRNA transcript encoding the STAG2 protein.

The protein expressed by the MTAP gene (SEQ ID NO:60) is also known as methylthioadenosine phosphorylase (MTAP). The expression level of a gene encoding MTAP can be measured using an oligonucleotide derived from SEQ ID NO:59 (GenBank Accession No. NM_{—}002451), the mRNA transcript encoding the MTAP protein.

In some embodiments of the invention, the nucleotide sequence of a suitable fragment of the gene is used, or an oligonucleotide derived thereof, to detect expression of the gene. The length of the oligonucleotide of any suitable length. A suitable length can be at least 10 nucleotides, 20 nucleotides, 50 nucleotides, 100 nucleotides, 200 nucleotides, or 400 nucleotides, and up to 500 nucleotides or 700 nucleotides. A suitable nucleotide is one which binds specifically to a nucleic acid encoding the target gene and not to the nucleic acid encoding another gene.

### Markers as Therapeutic Targets

In addition to determining the expression of the markers described herein to determine suitability of a cancer patient to conformationally-restricted polyamine treatment, it is contemplated that the genes described herein may also serve as therapeutic targets to render a cancer patient more suitable for treatment with conformationally-restricted polyamines. For example, methods to increase expression or activity of a protein encoded by a genes selected from the group consisting of PRKX, GABRP, FOXC1, EN1, RAD54B, STAG2, MTAP GCLM, LAMA3, SSRP1, ACYP1, CYLD, PRPF 18, AMFR, PPP1R2, and LOH11CR2A will render a patient more suitable to conformationally-restricted polyamine treatment. Conversely, methods to decrease expression or activity of a protein encoded by a gene selected from the group consisting of SCNN1A, CA12, TFF3, HNF3A, MYB, ESR1, AGR2, GATA3 RPL15, NEBL WASL, CST3, DEAF 1, and ACSL3 will render a patient more suitable for conformationally-restricted polyamine treatment. Methods to increase expression or activity of proteins encoded by genes include methods in gene therapy, such as viral expression, and compounds or small molecules that increase protein function. Methods to decrease expression or activity of proteins encoded by genes include use of neutralizing antibodies, siRNA/shRNA oligonucleotides, antisense oligonucleotide technology, small molecules that interfere with protein function, or an aptamer. The means to increase or decrease expression or activity of proteins encoded by the genes described herein comprises any means now known or developed in the future.

Examples of some of above-mentioned methods follow.

Antibodies: Polyclonal and monoclonal antibodies can be made by well-known methods in the art. A preferred method of generating these antibodies is by first synthesizing peptide fragments from an mRNA expressing a protein. Because synthesized peptides are not always immunogenic by their own, the peptides should be conjugated to a carrier protein before use. Appropriate carrier proteins include but are not limited to Keyhole limpet hemacyanin (KLH). The conjugated phosphor-peptides should then be mixed with adjuvant and injected into a mammal, preferably a rabbit through intradermal injection, to elicit an immunogenic response. Samples of serum can be collected and tested by ELISA assay to determine the titer of the antibodies and then harvested.

Polyclonal antibodies can be purified by passing the harvested antibodies through an affinity column. Monoclonal antibodies are preferred over polyclonal antibodies and can be generated according to standard methods known in the art of creating an immortal cell line which expresses the antibody. Nonhuman antibodies are highly immunogenic in humans and that limits their therapeutic potential. In order to reduce their immunogenicity, nonhuman antibodies need to be humanized for therapeutic application. Through the years, many researchers have developed different strategies to humanize the nonhuman antibodies. One such example is using “HuMAb-Mouse” technology available from MEDAREX, Inc. and disclosed by van de Winkel, in U.S. Pat. No. 6,111,166 and hereby incorporated by reference in its entirety. “HuMAb-Mouse” is a strain of transgenic mice which harbor the entire human immunoglobin (Ig) loci and thus can be used to produce fully human monoclonal antibodies such as monoclonal antibodies.

The antibodies generated in this manner can be used either to detected expression levels of the genes described herein or as a neutralizing antibody as described below.

Neutralizing antibodies: Polyclonal or monoclonal antibodies that specifically bind or inhibit the protein target, can be used using methods known in the art to neutralize the activity of the protein.

RNA interference (RNAi) sequence design: RNA interference is used to generate small double-stranded RNA (small interference RNA (siRNA) or short hairpin RNA (shRNA)) inhibitors to affect the expression of a candidate gene generally through cleaving and destroying its cognate RNA. Herein siRNA and shRNA may be used interchangeably. Small interference RNA (siRNA or shRNA) is typically 19-22 nt double-stranded RNA. siRNA can be obtained by chemical synthesis or by DNA-vector based RNAi technology. Using DNA vector based siRNA technology, a small DNA insert (about 70 bp) encoding a short hairpin RNA targeting the gene of interest is cloned into a commercially available vector. The insert-containing vector can be transfected into the cell, and expressing the short hairpin RNA. The hairpin RNA is rapidly processed by the cellular machinery into 19-22 nt double stranded RNA (siRNA). In a preferred embodiment, the siRNA is inserted into a suitable RNAi vector because siRNA made synthetically tends to be less stable and not as effective in transfection. siRNA can be made using methods and algorithms well known in the art.

siRNA are suggested to be built using the ORF (open reading frame) as the target selecting region, preferably 50-100 nt downstream of the start codon. Because siRNAs function at the mRNA level, not at the protein level, to design an siRNA, the precise target mRNA nucleotide sequence may be required. Due to the degenerate nature of the genetic code and codon bias, it is difficult to accurately predict the correct nucleotide sequence from the peptide sequence. Additionally, because the function of siRNAs is to cleave mRNA sequences, it is important to use the mRNA nucleotide sequence and not the genomic sequence for siRNA design.

Rational siRNA design should also minimize off-target effects which often arise from partial complementarity of the sense or antisense strands to an unintended target. These effects are known to have a concentration dependence and one way to minimize off-target effects is often by reducing siRNA concentrations. Another way to minimize such off -target effects is to screen the siRNA for target specificity.

The siRNA can be modified on the 5′-end of the sense strand to present compounds such as fluorescent dyes, chemical groups, or polar groups. Modification at the 5′-end of the antisense strand has been shown to interfere with siRNA silencing activity and therefore this position is not recommended for modification. Modifications at the other three termini have been shown to have minimal to no effect on silencing activity.

It is recommended that primers be designed to bracket one of the siRNA cleavage sites as this will help eliminate possible bias in the data (i.e., one of the primers should be upstream of the cleavage site, the other should be downstream of the cleavage site). Bias may be introduced into the experiment if the PCR amplifies either 5′ or 3′ of a cleavage site, in part because it is difficult to anticipate how long the cleaved mRNA product may persist prior to being degraded. If the amplified region contains the cleavage site, then no amplification can occur if the siRNA has performed its function.

Inhibitor Antisense Oligonucleotide: In another embodiment, antisense oligonucleotides (“oligos” and “oligomers”) can be designed to inhibit the gene markers described herein and other candidate gene function. Antisense oligonucleotides are short single-stranded nucleic acids, which function by selectively hybridizing to their target mRNA, thereby blocking translation. Translation is inhibited by either RNase H nuclease activity at the DNA-RNA duplex, or by inhibiting ribosome progression, thereby inhibiting protein synthesis. This results in discontinued synthesis and subsequent loss of function of the protein for which the target mRNA encodes.

In a preferred embodiment, antisense oligos are phosphorothioated upon synthesis and purification, and are usually 18-22 bases in length. It is contemplated that the antisense oligos may have other modifications such as 2′-O-Methyl RNA, methylphosphonates, chimeric oligos, modified bases and many others modifications, including fluorescent oligos.

Active antisense oligos should be compared against control oligos that have the same general chemistry, base composition, and length as the antisense oligo. These can include inverse sequences, scrambled sequences, and sense sequences. The inverse and scrambled are recommended because they have the same base composition, thus same molecular weight and Tm as the active antisense oligonucleotides. Rational antisense oligo design should consider, for example, that the antisense oligos do not anneal to an unintended mRNA or do not contain motifs known to invoke immunostimulatory responses such as four contiguous G residues, palindromes of 6 or more bases and CG motifs.

Antisense oligonucleotides can be used in vitro in most cell types with good results. However, some cell types require the use of transfection reagents to effect efficient transport into cellular interiors. It is recommended that optimization experiments be performed by using differing final oligonucleotide concentrations in the 1-5 μm range with in most cases the addition of transfection reagents. The window of opportunity, i.e., that concentration where you will obtain a reproducible antisense effect, may be quite narrow, where above that range you may experience confusing non-specific, non-antisense effects, and below that range you may not see any results at all. In a preferred embodiment, down regulation of the targeted mRNA will be demonstrated by use of techniques such as northern blot, real-time PCR, cDNA/oligo array or western blot. The same endpoints can be made for in vivo experiments, while also assessing behavioral endpoints.

For cell culture, antisense oligonucleotides should be re-suspended in sterile nuclease-free water (the use of DEPC-treated water is not recommended). Antisense oligonucleotides can be purified, lyophilized, and ready for use upon re-suspension. Upon suspension, antisense oligonucleotide stock solutions may be frozen at −20 2 C and stable for several weeks.

High throughput screening for small molecule inhibitors: In one embodiment, high throughput screening (HTS) methods are used to identify compounds that either inhibit or induce the genes described herein. HTS methods involve providing a combinatorial chemical or peptide library containing a large number of potential therapeutic compounds (i.e., compounds that either inhibit or induce gene expression). Such “libraries” are then screened in one or more assays to identify those library members (particular peptides, chemical species or subclasses) that display the desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries. Other chemistries for generating chemical diversity libraries can also be used. Such chemistries include, but are not limited to: peptioids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. ScL USA 90:6909-6913 (1993)), vinylogous polypeptides (Hagihara et al. , J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al, J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3). -309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., ECIS™, Applied BioPhysics Inc., Troy, N.Y., MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Recombinant expression, synthesis and isolation of gene inhibitors: Gene inhibitors such as the siRNA inhibitor described herein can also be made using nucleic acid or peptide synthesis or expressed recombinantly. The entire inhibitor sequence can be made using commercial oligonucleotide synthesis or peptide synthesis. The invention further contemplates the use of both native and modified DNA and RNA bases, e.g. beta-D-Glucosyl-Hydroxymethyluracil, and native and modified amino acid residues.

The nucleic acid sequences encoding gene inhibitors such as the siRNA inhibitor and related nucleic acid sequence homologues can be cloned. This aspect of the invention relies on routine techniques in the field of recombinant genetics. Generally, the nomenclature and the laboratory procedures in recombinant DNA technology described herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use in this invention include Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al. eds., 1994)). Substantially identical nucleic acids encoding sequences of gene inhibitors can be isolated using nucleic acid probes and oligonucleotides under stringent hybridization conditions, by screening libraries. Alternatively, expression libraries can be used to clone these sequences, by detecting expressed homologues immunologically with antisera or purified antibodies made against the core domain of nucleic acids encoding the gene inhibitor sequences.

Gene expression of the markers described herein can also be analyzed by techniques known in the art, e.g., reverse transcription and amplification of mRNA, isolation of total RNA or poly A+RNA, northern blotting, dot blotting, in situ hybridization, RNase protection, probing DNA microchip arrays, and the like.

To obtain high level expression of a cloned gene or nucleic acid sequence, such as those cDNAs encoding nucleic acid sequences encoding gene inhibitors such as the shRNA inhibitor and related nucleic acid sequence homologues, one typically subclones an inhibitor peptide sequence into an expression vector that is subsequently transfected into a suitable host cell. The expression vector typically contains a strong promoter or a promoter/enhancer to direct transcription, a transcription/translation terminator, and for a nucleic acid encoding a protein, a ribosome binding site for translational initiation. The promoter is operably linked to the nucleic acid sequence encoding the gene inhibitors. Suitable bacterial promoters are well known in the art and described, e.g., in Sambrook et al. and Ausubel et al. The elements that are typically included in expression vectors also include a replicon that functions in a suitable host cell such as E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to the recombinant gene inhibitors peptides to provide convenient methods of isolation, e.g., His tags. In some cases, enzymatic cleavage sequences (e.g., Met-(His)g-He-Glu-GLy-Arg which form the Factor Xa cleavage site) are added to the recombinant gene inhibitor peptides. Bacterial expression systems for expressing the gene inhibitor peptides and nucleic acids are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Standard transfection methods are used to produce cell lines that express large quantities of the gene inhibitor, which can then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of cells is performed according to standard techniques (see, e.g., Morrison, J. Bact. 132:349-351 (1977); Clark-Curtiss & Curtiss, Methods in Enzymology 101:347-362 (Wu et al, eds., 1983). For example, any of the well known procedures for introducing foreign nucleotide sequences into host cells may be used. These include the use of calcium phosphate transfection, lipofectamine, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the gene inhibitor peptides and nucleic acids.

After the expression vector is introduced into the cells, the transfected cells are cultured under conditions favoring expression of inhibitors such as the siRNA gene inhibitor and related nucleic acid sequence homologues.

Gene Therapy: In certain embodiments, the nucleic acids encoding inhibitory peptides and nucleic acids of the present invention can be used for transfection of cells in vitro and in vivo. These nucleic acids can be inserted into any of a number of well-known vectors for the transfection of target cells and organisms as described below. The nucleic acids are transfected into cells, ex vivo or in vivo, through the interaction of the vector and the target cell. The nucleic acid, under the control of a promoter, then expresses inhibitory peptides and nucleic acids of the present invention.

Alternatively, for genes for which increased expression may render a patient suitable for conformationally-restricted polyamine treatment, a nucleotide sequence encoding the relevant gene may be inserted into the gene therapy vector for overexpression of the protein.

For delivery of nucleic acids, viral vectors may be used. Suitable vectors include, for example, herpes simplex virus vectors as described in Lilley et al., Curr. Gene Ther. 1(4):339-58 (2001), alphavirus DNA and particle replicons as described in e.g., Polo et al., Dev. Biol. (Basel) 104:181-5 (2000), Epstein-Barr virus (EBV)-based plasmid vectors as described in, e.g., Mazda, Curr. Gene Ther. 2(3):379-92 (2002), EBV replicon vector systems as described in e.g., Otomo et al., J. Gene Med. 3(4):345-52 (2001), adeno-virus associated viruses from rhesus monkeys as described in e.g., Gao et al., PNAS USA. 99(18):11854 (2002), adenoviral and adeno-associated viral vectors as described in, e.g., Nicklin and Baker, Curr. Gene Ther. 2(3):273-93 (2002). Other suitable adeno-associated virus (AAV) vector systems can be readily constructed using techniques well known in the art (see, e.g., U.S. Pat. Nos. 5,173,414 and 5,139,941; PCT Publication Nos. WO 92/01070 and WO 93/03769). Additional suitable vectors include E1B gene-attenuated replicating adenoviruses described in, e.g., Kim et al., Cancer Gene Ther.9 (9):125-36 (2002) and non-replicating adenovirus vectors described in e.g., Pascual et al., J. Immunol. 160(9):4465-72 (1998). Exemplary vectors can be constructed as disclosed by Okayama et al. (1983) Mol. Cell. Biol. 3:280.

Molecular conjugate vectors, such as the adenovirus chimeric vectors described in Michael et al. (1993) J. Biol Chem. 268:6866-6869 and Wagner et al. (1992) Proc. Natl Acad. Sci. USA 89:6099-6103, can also be used for gene delivery according to the methods of the invention.

In one illustrative embodiment, retroviruses provide a convenient and effective platform for gene delivery systems. A selected nucleotide sequence encoding an inhibitory gene nucleic acid or polypeptide or a gene marker to be overexpressed can be inserted into a vector and packaged in retroviral particles using techniques known in the art. The recombinant virus can then be isolated and delivered to a subject. Suitable vectors include lentiviral vectors as described in e.g., Scherr and Eder, Curr. Gene Ther. 2(1):45-55 (2002). Additional illustrative retroviral systems have been described (e.g., U.S. Pat. No. 5,219,740; Miller and Rosman (1989) BioTechniques 7:980-990; Miller (1990) Human Gene Therapy 1:5-14; Scarpa et al. (1991) Virology 180:849-852; Burns et al (1993) Proc. Natl. Acad. ScL USA 90:8033-8037; and Boris-Lawrie and Temin (1993) Curr. Opin. Genet. Develop. 3:102-109.

Inhibitor aptamer sequence design: In another embodiment, aptamer sequences which bind to specific RNA or DNA sequences can be made. As used herein, the terms “aptamer (s)” or “aptamer sequence(s)” are meant to refer to single stranded nucleic acids (RNA or DNA) whose distinct nucleotide sequence determines the folding of the molecule into a unique three dimensional structure. Aptamers comprising 15 to 120 nucleotides can be selected in vitro from a randomized pool of oligonucleotides (10¹⁴-10¹⁵ molecules). Any aptamers of the invention as described herein further contemplates the use of both native and modified DNA and RNA bases, such as beta-D-Glucosyl-Hydroxymethyluracil.

Aptamer sequences can be isolated through methods such as those disclosed in U.S. Pat. No. 7,329,742, entitled, “Aptamers and Methods for their In vitro Selection and Uses Thereof,” which is hereby incorporated by reference.

It is contemplated that the sequences described herein may be varied to result in substantially homologous sequences which retain the same function as the original. As used herein, a polynucleotide or fragment thereof is “substantially homologous” (or “substantially similar”) to another if, when optimally aligned (with appropriate nucleotide insertions or deletions) with the other polynucleotide (or its complementary strand), using an alignment program such as BLASTN (Altschul, S. F., Gish, W., Miller, W., Myers, E. W. & Lipman, D J. (1990) “Basic local alignment search tool.” J. Mol. Biol. 215:403-410), and there is nucleotide sequence identity in at least about 80%, preferably at least about 90%, and more preferably at least about 95-98% of the nucleotide bases.

Nucleic acids encoding sequences of the gene inhibitors can also be isolated from expression libraries using antibodies as probes. Such polyclonal or monoclonal antibodies can be raised using, for example, the polypeptides comprising the even-numbered sequences set forth in SEQ ID NOS: 2-60, and subsequences thereof, using methods known in the art (see, e.g., Harlow and Lane, Antibodies: A Laboratory Manual (1988).

Methods of administration and treatment: The gene inhibitors of the present invention, such as the siRNA inhibitor, also can be used to treat or prevent a variety of disorders associated with cancer.

The antibodies, peptides and nucleic acids are administered to a patient in an amount sufficient to elicit a therapeutic response in the patient (e.g., inhibiting the development, growth or metastasis of basal cancerous cells; reduction of basal cell tumor size and growth rate, prolonged survival rate, reduction in concurrent cancer therapeutics administered to patient, enhanced activity of conformationally-restricted polyamines administered to a patient). An amount adequate to accomplish this is defined as “therapeutically effective dose or amount.”

The antibodies, peptides and nucleic acids of the invention can be administered directly to a mammalian subject using any route known in the art, including e.g., by injection (e.g., intravenous, intraperitoneal, subcutaneous, intramuscular, or intradermal), inhalation, transdermal application, rectal administration, or oral administration.

In other embodiments, such antibodies that specifically bind or inhibit target proteins, may be used therapeutically. Such use of antibodies has been demonstrated by others and may be useful in the present invention to inhibit or downregulate target protein markers.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Administration of the antibodies, peptides and nucleic acids of the invention can be in any convenient manner, e.g., by injection, intratumoral injection, intravenous and arterial stents (including eluting stents), catheter, oral administration, inhalation, transdermal application, or rectal administration. In some cases, the peptides and nucleic acids are formulated with a pharmaceutically acceptable carrier prior to administration. Pharmaceutically acceptable carriers are determined in part by the particular composition being administered (e.g., nucleic acid or polypeptide), as well as by the particular method used to administer the composition.

The present gene inhibitors may be administered singly or in combination, and may further be administered in combination with other anti-neoplastic drugs known and determined by those familiar with the art. They may be conventionally prepared with excipients and stabilizers in sterilized, lyophilized powdered form for injection, or prepared with stabilizers and peptidase inhibitors of oral and gastrointestinal metabolism for oral administration.

The pharmaceutical forms suitable for injectable use include sterile aqueous solutions or dispersions and sterile powders for the extemporaneous preparation of sterile injectable solutions or dispersions (U.S. Pat. No. 5,466,468). The carrier can be a solvent or dispersion medium containing, for example, water, ethanol, polyol (e.g., glycerol, propylene glycol, and liquid polyethylene glycol, and the like), suitable mixtures thereof, and/or vegetable oils.

The dose administered to a patient, in the context of the present invention should be sufficient to effect a beneficial therapeutic response in the patient over time. The dose will be determined by the efficacy of the particular vector (e.g. peptide or nucleic acid) employed and the condition of the patient, as well as the body weight or surface area of the patient to be treated. The size of the dose also will be determined by the existence, nature, and extent of any adverse side-effects that accompany the administration of a particular peptide or nucleic acid in a particular patient.

In determining the effective amount of the vector to be administered in the treatment or prophylaxis of diseases or disorder associated with the disease, the physician evaluates circulating plasma levels of the polypeptide or nucleic acid, polypeptide or nucleic acid toxicities, progression of the disease (e.g., ovarian cancer), and the production of antibodies that specifically bind to the peptide. Typically, the dose equivalent of a polypeptide is from about 0.1 to about 50 mg per kg, preferably from about 1 to about 25 mg per kg, most preferably from about 1 to about 20 mg per kg body weight. In general, the dose equivalent of a naked nucleic acid is from about 1 μg to about 100 μg for a typical 70 kilogram patient, and doses of vectors which include a viral particle are calculated to yield an equivalent amount of therapeutic nucleic acid.

For administration, antibodies, polypeptides and nucleic acids of the present invention can be administered at a rate determined by the LD₅₀ of the polypeptide or nucleic acid, and the side-effects of the antibody, polypeptide or nucleic acid at various concentrations, as applied to the mass and overall health of the patient. Administration can be accomplished via single or divided doses, e.g., doses administered on a regular basis (e.g., daily) for a period of time (e.g., 2, 3, 4, 5, 6, days or 1-3 weeks or more).

In certain circumstances it will be desirable to deliver the pharmaceutical compositions comprising the inhibitor antibodies, peptides and nucleic acids parenterally, intravenously, intramuscularly, or even intraperitoneally as described in U.S. Pat. No. 5,543,158; U.S. Pat. No. 5,641,515 and U.S. Pat. No. 5,399,363. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

Five to twenty micrograms of the present siRNA or antisense oligonucleotides can be suspended in 100 microliters of buffer such as PBS (phosphate buffered saline) for injecting into a subject intravenously to induce apoptosis of cancer cells. (See Slaton, Unger, Sloper, Davis, Ahmed, Induction of apoptosis by antisense CK2 in human prostate cancer xenograft model, Mol Cancer Res. 2004 December; 2(12):712-21.)

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologies standards.

The compositions may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

To date, most siRNA studies have been performed with siRNA formulated in sterile saline or phosphate buffered saline (PBS) that has ionic character similar to serum. There are minor differences in PBS compositions (with or without calcium, magnesium, etc.) and investigators should select a formulation best suited to the injection route and animal employed for the study. Lyophilized oligonucleotides and standard or stable siRNAs are readily soluble in aqueous solution and can be resuspended at concentrations as high as 2.0 mM. However, viscosity of the resultant solutions can sometimes affect the handling of such concentrated solutions.

Delivery of therapeutics: In certain embodiments, the use of liposomes, nanocapsules, microparticles, microspheres, lipid particles, vesicles, and the like, are contemplated for the administration of the inhibitory nucleic acids of the present invention. In particular, the compositions of the present invention may be formulated for delivery either encapsulated in or operatively attached to a lipid particle, a liposome, a vesicle, a nanosphere, or a nanoparticle or the like.

The formation and use of liposomes is generally known to those of skill in the art (see for example, Couvreur et at , 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon & Papahadjopoulos, 1988; Allen and Choun, 1987; U.S. Pat. No. 5,741,516). Further, various methods of liposome and liposome like preparations as potential drug carriers have been reviewed (Takakura, 1998; Chandran et al., 1997; Margalit, 1995; U.S. Pat. No. 5,567,434; U.S. Pat. No. 5,552,157; U.S. Pat. No. 5,565,213; U.S. Pat. No. 5,738,868 and U.S. Pat. No. 5,795,587).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 m. Sonication of MLVs results in the formation of small unilamellar vesicles (SUVs) with diameters in the range of 200 to 500 angstroms, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e., in the aqueous spaces and within the bilayer itself, respectively. It is possible that the drug-bearing liposomes may even be employed for site-specific delivery of active agents by selectively modifying the liposomal formulation.

Targeting is generally not a limitation in terms of the present invention. However, should specific targeting be desired, methods are available for this to be accomplished. For example, antibodies may be used to bind to the liposome surface and to direct the liposomes and its contents to particular cell types. Carbohydrate determinants (glycoprotein or glycolipid cell-surface components that play a role in cell-cell recognition, interaction and adhesion) may also be used as recognition sites as they have potential in directing liposomes to particular cell types.

Alternatively, the compounds can be delivered via pharmaceutically-acceptable nanocapsule formulations of the compositions of the present invention. Nanocapsules can generally entrap compounds in a stable and reproducible way (Henry-Michelland et al, 1987; Quintanar-Guerrero et al., 1998; Douglas et al., 1987). To avoid side effects due to intracellular polymeric overloading, such ultrafine particles (sized around 0.1 m) should be designed using polymers able to be degraded in vivo. Biodegradable polyalkyl-cyanoacrylate nanoparticles that meet these requirements are contemplated for use. Such particles may be easily made, as described (Couvreur et al, 1980; 1988; Muhlen et al , 1998; Zambaux et al 1998; Pinto-Alphandry et al, 1995 and U.S. Pat. No. 5,145,684). Others have described nanoparticles in U.S. Pat. Nos. 6,602,932; 6,071,533. It is further contemplated that the inhibitors of the present invention is delivered to cancerous cells in a subject using other microparticles, nanostructures and nanodevices. For example, microspheres may be used such as those available from PolyMicrospheres, Inc. (Indianapolis, Ind.). For descriptions of drug delivery, see generally Alivisatos AP, Less is more in medicine, Understanding Nanotechnology, Warner Books, New York, 2002; Max Sherman, The World of Nanotechnology, US Pharm. 2004; 12:HS-3-HS-4; Brannon-Peppas and Blanchette, Nanoparticle and targeted systems for cancer therapy, Advanced Drug Delivery Reviews, Intelligent Therapeutics: Biomimetic Systems and Nanotechnology in Drug Delivery, Volume 56, Issue 11, 22 Sep. 2004, Pages 1649-1659; and D. M. Brown, ed., Drug Delivery Systems in Cancer Therapy, Humana Press, Inc., Totowa, N.J. 2004, including Chapter 6: Microparticle Drug Delivery Systems by Birnbaum and Brannon-Peppas, pp. 117-136, all of which are hereby incorporated by reference.

### Conformationally Restricted Polyamines: Synthetic Approach

The manufacture of bioactive spermine ligands which may affect the structure of chromatin can be illustrated by the introduction of cyclopropyl and cyclobutyl constraints into the flexible spermine molecule. Spermine appears as follows:

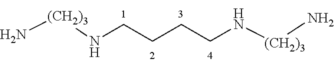

The first targeted location was the central 1,4-diaminobutane segment. In its staggered conformation, four semi-eclipsed conformational rotamers are possible around the diaminobutane segment. The four have enantiomeric relationships. Introduction of a bond between the C-1 and C-3 positions or the C-2 and C-4 positions of the central diaminobutane segment generates a cyclopropane ring. Introduction of an additional bond between the C-2 and C-3 positions generates a conformationally restricted alkene derivative. Cyclobutyl, cyclopentyl, and cyclohexyl moieties can be introduced into the structure following the same strategy.

Using this approach four conformationally semi-rigid structures were obtained which mimic the four semi-eclipsed conformational structures of spermine Two of the semi-rigid structures are epimers of the other two.

For purposes of the present invention, it is important to note that the cis and trans isomers of the subject compounds assume very distinct three-dimensional conformations due to the restricted bond rotation afforded by the centrally-located ring structure or unsaturation. All geometric isomers (optically active or otherwise), including pure isolated cis forms and pure isolated trans forms of the subject compounds, and mixtures thereof, are explicitly within the scope of the present invention. Additionally, all positional isomers of the subject compounds are explicitly within the scope of the present invention. When A or D is a cyclical moiety, the two B substituents or the amino moieties, respectively, may be oriented in the 1,2 or 1,3 or 1,4 position with respect to each other.

Following the protocols as presented in U.S. Pat. No. 5,899,061, and using suitable and well known starting reagents, all of the compounds of Formula I, including those where A and D are independently C₅ or C₆ cycloalkyl, cycloalkenyl, or cycloaryl, can be readily obtained.

The pure compounds, as well as pharmaceutically-suitable salts thereof, are explicitly within the scope of the above-noted compounds. By the term “pharmaceutically-suitable salts” is meant any salt form of the subject compounds which renders them more amenable to administration by a chosen route. A wide range of such salts are well known to those of skill in the pharmaceutical art. The preferred pharmaceutically-suitable salts are acid addition salts such as chlorides, bromides, iodides and the like.

### Utility of the Conformationally Restricted Polyamines as General Anti-Neoplastic Agents

To assess the utility of the subject compounds in the treatment of neoplastic cell growth, the ability of the compounds to inhibit the in vitro growth of several commonly used cancer models was studied. The subject polyamines induce cell death in several neoplastic cell lines at drug concentrations smaller than 10 μM. In serial dilution, the restricted conformation polyamines of the present invention have been shown to inhibit cell growth and/or cause cell death in accepted in vitro test cultures for human breast cancer (MCF7), brain cancer (U251MG NCI), lung cancer (A549), colon cancer (HT29), and prostate cancer (PC3) at minute concentrations heretofore undescribed in the scientific literature. See U.S. Pat. No. 5,889,061, incorporated herein by reference.

### Administration and Pharmaceutical Unit Dosage Forms

The above-described compounds being effective to inhibit the growth of cancer cells, the compounds are suitable for the therapeutic treatment of neoplastic conditions in mammals, including humans. Cancer cell growth inhibition at pharmacologically-acceptable concentrations has been shown in human breast cancer, brain cancer, lung cancer, colon cancer, and prostate cancer cell lines.

Administration of the subject conformationally restricted polyamines to a human or non-human patient can be accomplished by any means known. The preferred administration route is parenteral, including intravenous administration, intra-arterial administration, intra-tumor administration, intramuscular administration, intraperitoneal administration, and subcutaneous administration in combination with a pharmaceutical carrier suitable for the chosen administration route. The treatment method is also amenable to oral administration.

It must be noted, as with all pharmaceuticals, the concentration or amount of the polyamine administered will vary depending upon the severity of the ailment being treated, the mode of administration, the condition and age of the subject being treated, and the particular polyamine or combination of polyamines being used.

The compounds described herein are administratable in the form of tablets, pills, powder mixtures, capsules, injectables, solutions, suppositories, emulsions, dispersions, food premixes, and in other suitable forms. The pharmaceutical dosage form which contains the compounds described herein is conveniently admixed with a non-toxic pharmaceutical organic carrier or a non-toxic pharmaceutical inorganic carrier. Typical pharmaceutically-acceptable carriers include, for example, mannitol, urea, dextrans, lactose, potato and maize starches, magnesium stearate, talc, vegetable oils, polyalkylene glycols, ethyl cellulose, poly(vinylpyrrolidone), calcium carbonate, ethyl oleate, isopropyl myristate, benzyl benzoate, sodium carbonate, gelatin, potassium carbonate, silicic acid, and other conventionally employed acceptable carriers. The pharmaceutical dosage form may also contain non-toxic auxiliary substances such as emulsifying, preserving, or wetting agents, and the like.

Solid forms, such as tablets, capsules and powders, can be fabricated using conventional tabletting and capsule-filling machinery, which is well known in the art. Solid dosage forms may contain any number of additional non-active ingredients known to the art, including excipients, lubricants, dessicants, binders, colorants, disintegrating agents, dry flow modifiers, preservatives, and the like.

Liquid forms for ingestion can be formulated using known liquid carriers, including aqueous and non-aqueous carriers, suspensions, oil-in-water and/or water-in-oil emulsions, and the like. Liquid formulation may also contain any number of additional non-active ingredients, including colorants, fragrance, flavorings, viscosity modifiers, preservatives, stabilizers, and the like.

For parenteral administration, the subject compounds may be administered as injectable dosages of a solution or suspension of the compound in a physiologically-acceptable diluent or sterile liquid carrier such as water or oil, with or without additional surfactants or adjuvants. An illustrative list of carrier oils would include animal and vegetable oils (peanut oil, soy bean oil), petroleum-derived oils (mineral oil), and synthetic oils. In general, for injectable unit doses, water, saline, aqueous dextrose and related sugar solutions, and ethanol and glycol solutions such as propylene glycol or polyethylene glycol are preferred liquid carriers.

The pharmaceutical unit dosage chosen is preferably fabricated and administered to provide a concentration of drug at the point of contact with the cancer cell of from 1 μM to 10 mM. More preferred is a concentration of from 1 to 100 μM. This concentration will, of course, depend on the chosen route of administration and the mass of the subject being treated.

### EXAMPLES

### Example 1

### Markers of Basal or Luminal Cancer Cell Subtypes

Analyses of expression profiles have been particularly powerful in identifying distinctive breast cancer subsets that differ in biological characteristics and clinical outcome (Perou et al., 1999, 2000; Sorlie et al., 2001, 2003). For example, unsupervised hierarchical clustering of microarray-derived expression data has identified intrinsically variable gene sets that distinguish five breast cancer subtypes—basal-like, luminal A, luminal B, ERBB2, and normal breast-like. The basal-like and ERBB2 subtypes have been associated with strongly reduced survival durations in patients treated with surgery plus radiation (Perou et al., 2000; Sorlie et al., 2001), and some studies have suggested that reduced survival duration in poorly performing subtypes is caused by an inherently high propensity to metastasize (Ramaswamy et al., 2003). These analyses already have led to the development of multigene assays that stratify patients into groups that can be offered treatment strategies based on risk of progression (Esteva et al., 2005; Gianni et al., 2005; van't Veer et al., 2002; van de Vijver et al., 2002). However, the predictive power of these assays is still not as high as desired, and the assays have not been fully tested in patient populations treated with aggressive adjuvant chemotherapies.

We have extended these studies by performing combined analyses of genome copy number and gene expression to identify genes that contribute to breast cancer pathophysiology, with emphasis on those that are associated with poor response to current therapies. Specifically, we determined expression markers that identify cells as possessing either a basal or luminal subtype and determined the sensitivity of these subtypes to conformationally-restrictive polyamines

We assessed genome copy number using BAC array CGH (Hodgson et al., 2001; Pinkel et al., 1998; Snijders et al., 2001; Solinas-Toldo et al., 1997) and gene expression profiles using Affymetrix U133A arrays (Ramaswamy et al., 2003; Reyal et al., 2005) in breast tumors from a cohort of patients treated according to the standard of care between 1989 and 1997 (surgery, radiation, hormonal therapy, and treatment with high-dose adriamycin and cytoxan as indicated). We measured genome copy number profiles for 145 primary breast tumors and gene expression profiles for 130 primary tumors, of which 101 were in common. We also measured gene expression profiles in 48 breast cancer cell lines. We analyzed these data to identify recurrent genomic and transcriptional abnormalities, and we assessed associations with clinical endpoints to identify genomic events that might contribute to cancer pathophysiology and sensitivity to pharmaceutical agents. The methods used are described below and in Neve et al. (2006).

Cell culture: Breast cancer cell lines were obtained from the ATCC or from collections developed in the laboratories of Drs. Steve Ethier and Adi Gazdar. Cell lines were obtained from these sources to avoid errors that occur when obtaining lines through “secondhand” sources. Because we acknowledge the existence of multiple clonal variants of some cell lines throughout the scientific community, all results presented here are reflective of the cell lines we have in our collection. To maintain the collections' integrity, cell lines have been carefully maintained in culture, and stocks of the earliest-passage cells have been stored. Quality control is maintained by careful analysis and reanalysis of morphology, growth rates, gene expression, and protein levels. Cell lines can be accurately identified by CGH analysis. All extracts were made from subconfluent cells in the exponential phase of growth in full media. Information about the biological characteristics of the cell lines and the culture conditions are summarized in and are available at http://cancer.lbl.gov/breastcancer/data.php.

Nucleic acid isolation; DNA isolation: Cells growing exponentially in culture were washed in phosphate buffered saline (PBS), pelleted by centrifugation, resuspended in PBS, and pelleted again. Pellets were either frozen for long-term storage or used to extract genomic DNA directly. Genomic DNA was extracted using the Wizard DNA Purification Kit (Promega), further purified with a phenol/chloroform extraction, and quantified using a fluorimeter. Phenol/chloroform extraction of the resulting DNA increased measurement precision significantly in some experiments, presumably by removing proteins that interfered with DNA labeling and hybridization.

RNA isolation: Total RNA was extracted from cell lines using Trizol, according to standard protocols (Invitrogen). RNA integrity was assessed by denaturing formaldehyde agarose gel electrophoresis or by microanalysis (Agilent Bioanalyzer, Palo Alto, Calif.).

Cell lysates: Protein lysates were prepared from cells at 50%-75% confluency. The cells were washed in ice-cold PBS containing 1 mM phenylmethylsulfonyl fluoride (PMSF) and then with a buffer containing 50 mM HEPES (pH 7.5), 150 mM NaCl, 25 mM b-glycerophosphate, 25 mM NaF, 5 mM EGTA, 1 mM EDTA, 15 mM pyrophosphate, 2 mM sodium orthovanadate, 10 mM sodium molybdate, leupeptin (10 mg/ml), aprotinin (10 mg/ml), and 1 mM PMSF. Cells were extracted in the same buffer containing 1% Nonidet-P40. Lysates were then clarified by centrifugation and frozen at 280° C. Protein concentrations were determined using the Bio-Rad protein assay kit.

Protein quantification: Protein levels were measured by quantifying emitted chemiluminescence or infrared radiation recorded from labeled antibodies using Scion Image (http://www.scioncorp.com/) or Odyssey software (http://www.licor.com/). For each protein, the blots were made for 4 sets of 11 cell lines, each set including the same pair (SKBR3 and MCF12A) to permit intensity normalization across sets. A basic multiplicative normalization was carried out by fitting a linear mixed-effects model to log intensity values and adjusting within each set to equalize the log intensities of the pair of reference cell lines across the sets.

Comparative genomic hybridization: Each sample was analyzed using Scanning and OncoBAC arrays. Scanning arrays were comprised of 2464 BACs selected at approximately megabase intervals along the genome as described previously (Hodgson et al., 2001; Snijders et al., 2001). OncoBAC arrays were comprised of 1860 P1, PAC, or BAC clones. About three-quarters of the clones on the OncoBAC arrays contained genes and STSs implicated in cancer development or progression. All clones were printed in quadruplicate. Data presented are the union of these two data sets. Arrays were prepared as described (Fridlyand et al., 2006; Snijders et al., 2001). Briefly, we random-prime labeled 500w1000 ng of test (cell line) and reference (normal female, Promega) genomic DNA with CY3-dUTP and CY5-dUTP (Amersham), respectively, using Bioprime kit (Invitrogen). Labeled DNA samples were co-precipitated with 50 mg of human Cot-1 DNA (Invitrogen), denatured, hybridized to BAC arrays for 48-72 hr, washed, and counterstained with DAPI. Most of the data presented are based on the results of a single hybridization. Repeated measurements of genome aberrations in other experiments show that the results are highly reproducible.

Data processing for CGH data: Array CGH data image analyses were performed as described previously (Jain et al., 2002). In this process, an array probe was assigned a missing value for an array if there were fewer than two valid replicates or the standard deviation of the replicates exceeded 0.3. Array probes missing in more than 50% of samples in the OncoBAC or scanning array data sets were excluded in the corresponding set. Array probes representing the same DNA sequence were averaged within each data set and then between the two data sets. Finally, the two data sets were combined, and the array probes missing in more than 25% of the samples, unmapped array probes, and probes mapped to chromosome Y were eliminated. The final data set contained 2696 unique probes representing a resolution of 1 Mb.

Affymetrix microarray analysis: Total RNA was prepared from samples using Trizol reagent (GIBCO BRL Life Technologies), and quality was assessed on the Agilent Bioanalyser 2100. Preparation of in vitro transcription (IVT) products, oligonucleotide array hybridization, and scanning were performed according to Affymetrix (Santa Clara, Calif.) protocols. In brief, 5 mg of total RNA from each breast cancer cell line and T7-linked oligo-dT primers were used for first-strand cDNA synthesis. IVT reactions were performed to generate biotinylated cRNA targets, which were chemically fragmented at 95° C. for 35 min. Fragmented biotinylated cRNA (10 mg) was hybridized at 45° C. for 16 hr to Affymetrix high density oligonucleotide array human HG-U133A chip. The arrays were washed and stained with streptavidin-phycoerythrin (SAPE; final concentration 10 mg/ml). Signal amplification was performed using a biotinylated anti-streptavidin antibody. The array was scanned according to the manufacturer's instructions (2001 Affymetrix Genechip Technical Manual). Scanned images were inspected for the presence of obvious defects (artifacts or scratches) on the array. Defective chips were excluded, and the sample was reanalyzed.

Data processing: Probe set based gene expression measurements were generated from quantified Affymetrix image files (“.CEL” files) using the RMA algorithm (Irizarry et al., 2003) from the BioConductor (http://www.bioconductor.org/) tools suite. All 51 CEL files were analyzed simultaneously, creating a data matrix of probe sets by cell lines in which each value is the calculated log abundance of each probe set gene for each cell line. Probe sets were annotated with Unigene annotations from the July 2003 mapping of the human genome (http://genome.ucsc.edu/), resulting in 19,764 annotated probe sets representing 13,406 unique unigenes (Table 3). Gene expression values were centered by subtracting the mean value of each probe set across the cell line set from each measured value. The gene expression data were organized using hierarchical clustering to facilitate visualization of commonalities and differences in gene expression across the set of cell lines. These analyses were restricted to the set of genes that showed substantial variation across the data set by selecting all probe sets that had at least four measurements that varied by more than Log₂ 1.89. This resulted in 1438 probe sets corresponding to 1213 unigenes. This variation restriction was arbitrary but did not affect the outcome of the eventual analysis. Probe sets corresponding to the same gene were down-weighted inversely proportional to their frequency prior to clustering (Wouters et al., 2003). Agglomerative clustering (Eisen et al., 1998) was applied to probe sets and cell lines using the uncentered Pearson's correlations. Resulting clusters were visualized using Java TreeView (Saldanha, 2004).

TABLE 3

| GeneID | Unigene ID | GeneID | Unigene ID |
|---|---|---|---|
| SCNN1A | Hs.2794 | STAG2 | Hs.8217 |
| CA12 | Hs.5338 | MTAP | Hs.152817 |
| PRKX | Hs.147996 | WASL | Hs.143728 |
| TFF3 | Hs.352107 | GCLM | Hs.315562 |
| HNF3A | Hs.299867 | CST3 | Hs.304682 |
| MYB | Hs.1334 | LAMA3 | Hs.436367 |
| GABRP | Hs.70725 | SSRP1 | Hs.523680 |
| ESR1 | Hs.1657 | ACYP1 | Hs.18573 |
| AGR2 | Hs.91011 | CYLD | Hs.578973 |
| GATA3 | Hs.169946 | PRPF18 | Hs.161181 |
| FOXC1 | Hs.284186 | AMFR | Hs.295137 |
| EN1 | Hs.271977 | DEAF1 | Hs.243994 |
| RPL15 | Hs.74267 | PPP1R2 | Hs.535731 |
| RAD54B | Hs.128501 | LOH11CR2A | Hs.152944 |
| NEBL | Hs.5025 | ACSL3 | Hs.471461 |

PAM analysis: Analysis was performed in R (http://www-stat.stanford.edu/%7Etibs/ PAM/Rdist/index.html) following the instructions therein (http://www-stat.stanford.edu/%7Etibs/PAM/Rdist/doc/readme.html) (Tibshirani et al., 2002). Three classifiers were defined (luminal, Basal A, and Basal B, as determined from the hierarchical clustering of the cell line expression data). Classifier training, cross-validation, and calculation of false discovery rates were performed.

Association of copy number with expression: The presence of an overall dosage effect was assessed by subdividing each chromosomal arm into non-overlapping 20 Mb bins and computing the average of cross-Pearson's-correlations for all gene-clone pairs that mapped to that bin. The average cross-correlations between clones and genes mapping to the same bin were significantly higher than those between clones and genes mapping to unlinked bins (p value<10⁻¹⁶, Wilcoxon rank sum test). Pearson's correlations and corresponding p values between expression level and copy number also were calculated for each gene. Each gene was assigned an observed copy number of the nearest mapped BAC array probe. Eighty percent of genes had a nearest clone within 1 Mbp, and 50% had a clone within 400 kb. Correlation between expression and copy number was only computed for the mapped genes whose absolute assigned copy number exceeded 0.2 in at least five samples. This was done to avoid spurious correlations in the absence of real copy number changes. The Holm p value adjustment was applied to correct for multiple testing. Genes with an adjusted p value<0.05 were considered to have expression levels that were significantly affected by gene dosage. This corresponded to a minimum Pearson's correlation of 0.44.

Results: The expression levels of several genes were found to predict either a basal or luminal phenotype of a cell. The basal/luminal markers are the following genes: SCNN1A, CA12, PRKX, TFF3, HNF3A, MYB, GABRP, ESR1, AGR2, GATA3, FOXC1, and EN1 and their predictive expression levels are shown in Table 4.

TABLE 4

| | Affymetrix | Expression | Expression | | | GenBank |
|---|---|---|---|---|---|---|
| | Probe | in Luminal | in Basal | | Dist | Accession |
| Gene ID | ID | Cells | Cells | Chr. | (Mb) | No. (mRNA) |
| SCNN1A | 203453_at | High | Low | 12 | 1949.8 | NM_001038 |
| CA12 | 203963_at | High | Low | 15 | 2355.3 | NM_001218 |
| PRKX | 204061_at | Low | High | 23 | 2869.3 | NM_005044 |
| TFF3 | 204623_at | High | Low | 21 | 2812.4 | NM_003226 |
| HNF3A | 204667_at | High | Low | 14 | 2224.7 | NM_004496 |
| MYB | 204798_at | High | Low | 6 | 1197.3 | NM_005375 |
| GABRP | 205044_at | Low | High (BaA) | 5 | 1051.0 | NM_014211 |
| ESR1 | 205225_at | High | Low | 6 | 1214.0 | NM_000125 |
| AGR2 | 209173_at | High | Low | 7 | 1249.3 | NM_006408 |
| GATA3 | 209604_s_at | High | Low | 10 | 1682.1 | NM_002051, |
| | | | | | | NM_032742 |
| FOXC1 | 213260_at | Low | High | 6 | 1063.4 | AK021858 |
| EN1 | 220559_at | Low | High | 2 | 365.8 | NM_001426 |

As seen in Table 4, it was found that an increase in gene expression levels of PRKX, GABRP, FOXC1, and EN1 and/or a decrease in expression levels of SCNN1A, CA12, TFF3, HNF3A, MYB, ESR1, AGR2, and GATA3 indicate a basal-like phenotype. Furthermore, the inverse, wherein a decrease in gene expression levels of PRKX, GABRP, FOXC1, and EN1 and/or an increase in expression levels of SCNN1A, CA12, TFF3, HNF3A, MYB, ESR1, and AGR2 indicates a luminal phenotype.

The markers were validated against data from the Netherlands Cancer Institute (NKI, Amsterdam, Netherlands). The results are shown in Table 5.

TABLE 5

| | |
|---|---|
| UCSF Training | Netherlands |
| Cells | Cancer Institute |

| | Non-basal | Basal | Non-basal | Basal |
|---|---|---|---|---|
| Predicted | 98 | 2 | 244 | 6 |
| non-basal | | | | |
| Predicted basal | 0 | 30 | 5 | 40 |

### Example 2

### Effect of Conformationally-Restricted Polyamine on Basal versus Cancer Cell Subtypes

To better delineate the response of breast cancer cells to conformationally-restricted polyamines, we studied the anti-proliferation activity of CGC-11047 (SL-11047 or Compound 58 in Table 1) among the panel of 42 breast cancer cell lines and 6 non-cancerous breast cell lines (e.g., human mammary epithelial cells, HMEC) of known subtype (basal or luminal) with a plethora of genomic background mimicking the human breast tumors. Extensive genomic background of these cell lines have been reported by Neve et al. (2006).

Cell culture: Breast cell lines were obtained from the ATCC and from collections developed in the laboratories of Drs. Steve Ethier and Adi Gazdar.

Cell growth inhibition assay and data analysis: Cells were plated at proper density in 96-well plates such that they would remain in log growth at the end of assay time. The cells were allowed to attach overnight before being exposed to CGC-11047 for 72 h. Drugs were dissolved in water as 100 mM stock. For the dose response study, a set of 9 doses from 5×10⁻³ to 1.3×10⁻⁸ M (final concentration) in 1:5 serial dilution were added in triplicate wells. The final DMSO concentration in the treated well was 0.3% or less. The cell growth was determined using Cell Titer Glo (CTG) assay (CellTiter-Glo Luminescent Cell Viability Assay, Promega, Madison, Wis.), with slight modification form manufacturer's protocol, at day 0 (time when drug was added) and at day 3 of drug exposure. Briefly, CTG reagent was diluted with PBS (1:1, volume: volume) and the culture media was removed from the 96-well plate prior to adding 50 μl per well of the diluted CTG reagent. Luminescence from the assay was recorded using BIO-TEK FLx800.

Data calculations were made according to the method described by the NCI/NIH DTP Human Tumor Cell Line Screen Process (http://dtp.nci.nih.gov/branches/btb/ivclsp.html) and as described previously (Monks, A et al., JNCI 83:757-766, 1991). The percent growth curve is calculated as [(T−T₀)/(C−T₀)]×100, where T₀ is the cell count at day 0, C is the vehicle control (e.g. 0.3% DMSO without drug) cell count at day 3, and T is the cell count at the test concentration. The GI₅₀ and TGI value are determined as the drug concentration that results in a 50% and 0% growth at 72 h drug exposure. LC₅₀ is calculated as [(T−T₀)/T₀]×100=−50, when T<T₀.

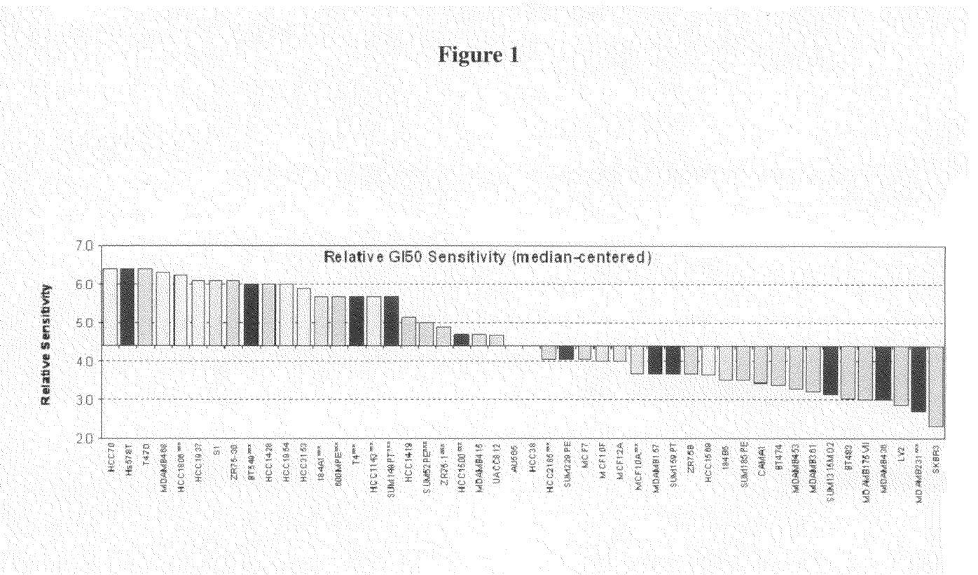

Results: Breast cancer cell lines responded to CGC-11047 treatment by resulting in reduced cell growth compared to untreated controls in a dose dependent fashion. The GI₅₀ range of the cell lines tested ranged between 0.4 uM to 5 mM, with a median GI₅₀ at around 40 uM. Cell lines were plotted by their GI₅₀ sensitivity to CGC-11047 to show the distribution of cell lines with respect to their sensitivity to treatment (FIG. 1 and Table 6). This arrangement revealed sensitivity to CGC-11047 treatment (i.e., lower GI₅₀) among cell lines of basal subtype (most of basal A and some of basal B) and resistance to the compound among cell lines of luminal subtype.

TABLE 6

| Cell Line | Subtype | GI₅₀ (M) | TGI (M) |
|---|---|---|---|
| HCC70 | Basal A | 4.0E−07 | 5.3E−07 |
| Hs578T | Basal B | 4.0E−07 | 5.0E−04 |
| T47D | Luminal | 4.0E−07 | 2.0E−04 |
| MDAMB468 | Basal A | 5.0E−07 | 2.0E−03 |
| HCC1806 | Basal A | 6.0E−07 | 3.3E−04 |
| HCC1937 | Basal A | 8.0E−07 | 3.0E−04 |
| S1 | HMEC | 8.0E−07 | 3.0E−03 |
| ZR7530 | Luminal | 8.0E−07 | 1.0E−03 |
| BT549 | Basal B | 1.0E−06 | 1.3E−03 |
| HCC1428 | Luminal | 1.0E−06 | 3.0E−04 |
| HCC1954 | Basal A | 1.0E−06 | 8.0E−06 |
| HCC3153 | Basal A | 1.3E−06 | 8.0E−06 |
| 184A1 | HMEC | 2.0E−06 | 2.2E−04 |
| 600MPE | Luminal | 2.0E−06 | 2.0E−03 |
| T4 | Basal B | 2.0E−06 | 8.0E−04 |
| HCC1143 | Basal A | 2.0E−06 | 3.2E−04 |
| SUM149PT | Basal B | 2.0E−06 | 8.0E−04 |
| HCC1419 | Luminal | 7.0E−06 | 3.1E−05 |
| SUM52PE | Luminal | 1.0E−05 | 3.0E−04 |
| ZR751 | Luminal | 1.3E−05 | 8.0E−04 |
| HCC1500 | Basal B | 2.0E−05 | 6.0E−04 |
| MDAMB415 | Luminal | 2.0E−05 | 1.0E−04 |
| UACC812 | Luminal | 2.1E−05 | 3.0E−04 |
| AU565 | Luminal | 4.0E−05 | >3e−5 |
| HCC38 | Basal B | 4.0E−05 | 3.0E−04 |
| HCC2185 | Luminal | 9.0E−05 | 5.0E−04 |
| SUM229PE | Basal B | 9.0E−05 | 1.1E−03 |
| MCF7 | Luminal | 9.0E−05 | 1.2E−03 |
| MCF10F | N, Basal A | 1.0E−04 | 1.1E−03 |
| MCF12A | N, Basal B | 1.0E−04 | >1e−4 |
| MCF10A | N, Basal B | 2.0E−04 | 3.3E−03 |
| MDAMB157 | Basal B | 2.0E−04 | 1.0E−03 |
| SUM159PT | Basal B | 2.0E−04 | 3.0E−03 |
| ZR75B | Luminal | 2.0E−04 | 2.0E−03 |
| HCC1569 | Basal A | 2.2E−04 | 2.0E−03 |
| 184B5 | HMEC | 3.0E−04 | 4.0E−04 |
| SUM185PE | Luminal | 3.0E−04 | >5e−4 |
| CAMA1 | Luminal | 3.5E−04 | 1.1E−03 |
| BT474 | Luminal | 4.0E−04 | 1.0E−03 |
| MDAMB453 | Luminal | 5.0E−04 | >5e−4 |
| MDAMB361 | Luminal | 6.0E−04 | 2.0E−03 |
| SUM1315MO2 | Basal B | 7.0E−04 | 1.3E−03 |
| BT483 | Luminal | 9.0E−04 | 1.3E−03 |
| MDAMB175VII | Luminal | 1.0E−03 | 2.0E−03 |
| MDAMB436 | Basal B | 1.0E−03 | 3.0E−03 |
| LY2 | Luminal | 1.3E−03 | 3.0E−03 |
| MDAMB231 | Basal B | 2.0E−03 | >5e−3 |
| SKBR3 | Luminal | 5.0E−03 | >5e−3 |

These analyses demonstrated that CGC-11047 is more cytotoxic in cell lines representing clinically aggressive basal B breast cancers subtype than in luminal subtype cell lines or non-transformed human mammary epithelial cultures. Thus, the markers that differentiate basal versus luminal cancer subtypes (e.g., SCNN1A, CA12, PRKX, TFF3, HNF3A, MYB, GABRP, ESR1, AGR2, GATA3, FOXC1, and EN1) can serve as predictive markers to conformationally-restricted polyamines such as CGC-11047.

### Example 3

### Predictive Markers to Predict Sensitivity or Resistance to Conformationally-Restricted Polyamines

The GI₅₀ study as described in Example 2 was used to determine predictive markers for sensitivity or resistance to treatment with conformationally-restricted polyamines, specifically, CGC-11047. This was performed through genome-wide correlation of mRNA levels as determined through a gene expression array with the measured GI₅₀ values.

Affymetrix microarray analysis: The determination of gene expression (mRNA) levels was performed with a Affymetrix high density oligonucleotide array human HG-U133A chip as described in Example 1, except that the statistical analysis was performed by adaptive linear spline method.

Adaptive spline analysis: Adaptive linear splines proceed by searching for optimal partitions in the parameter space, characteristic of multiple classes, and fitting a linear model within each partition. The fitted function is continuous, resulting in a single optimization problem. Thus, adaptive splines can simultaneously account for class information and magnitude of response in a single framework. Briefly, the response data is expressed as a sum of linear splines, where the predictor variable is the specific molecular profile of the candidate marker. For a fixed number of knots, which define the partitions, the algorithm enumeratively searches for the best location of knots by minimizing the residual sum of squares. A central challenge in predicting response in small N (cell-lines), large P (predictors) problems, is that the noise can be very strong leading to over-fitting problems. We controlled for this by using leave-one-out cross-validation (LOOCV). We also used LOOCV to determine the optimal model size, i.e. the number of knots. Goodness of fit was assessed by computing the p-value corresponding to an F-statistic.

The genes that were determined to change in expression in response to CGC-11047 are shown in Table 7.

TABLE 7

| | | Predicts | | |
|---|---|---|---|---|
| | | Sensitivity (S) | Adaptive | GenBank |
| | Affymetrix | or Resistance | Spline p- | Accession No. |
| Gene ID | Probe ID | (R) | value | (mRNA) |
| RPL15 | 221476_s_at | R | 3.94E−07 | AF279903 |
| RAD54B | 219494_at | S | 5.51E−06 | NM_012415 |
| NEBL | 203962_s_at | R | 8.59E−06 | NM_006393 |
| STAG2 | 209023_s_at | S | 1.74E−05 | BC001765 |
| MTAP | 211363_s_at | S | 1.81E−05 | AF109294 |

According to Table 7, the following biomarkers are predictive of sensitivity to a conformationally-restricted polyamine: RAD54B, STAG2, and MTAP. The following biomarkers are predictive of resistance to a conformationally-restricted polyamine: RPL15 and NEBL.

### Example 4

### Additional Predictive Markers to Predict Sensitivity or Resistance to Conformationally-Restricted Polyamines

Using the same methods as in Example 3, additional biomarkers predicting sensitivity or resistance to conformationally-restricted polyamines in cancer cells were determined. Additional assays and analyses were performed as described below.

BrdU cell staining and labeling: After incubation of cells with CGC-11047 at specified concentrations for 24, 48, and 72 hours, cells were pulsed with a final BrdU concentration of 10 uM for 30 minutes. Cells were then fixed with −20° C. 70% ethanol overnight. Fixed cells were incubated with 2N HCl for 5-7 minutes to denature DNA. Cell were then rinsed with 1× PBS to neutralize HCl.

Cells were incubated with primary donkey anti-BrdU antibody (diluted 1:100 in 0.5% PBS-tween20) for 1 hour then rinsed well with 0.5% PBS-tween20. Cells were incubated with Hoechst 33642 (diluted 1:2000) and secondary anti-donkey antibody (diluted 1:500) for 45 minutes and washed well with PBS-tween. PBS added to cells in preparation for scan.

Caspase Glo assay: Apoptosis was measured using the Promega Caspase Glo 3/7 assay (Promega, Madison, Wis.). Lyophilized pellet was eluted in solvent to make working Caspase Glo reagent. After incubation of cells with CGC-11047 at specified concentrations for 24, 48, and 72 hours, 50 ul of Caspase Glo reagent was added to the wells. After an incubation time of 1 hour, luminescence was measured using a luminometer.

Results: An association study of based on GI₅₀ values and differentially expressed genes in the cell line panel upon treatment with CGC-11047 showed 250 genes with p<0.0034 and FDR<5.7. Most of these genes were involved in cell differentiation, apoptosis, response to stimulus and cell motility. Further analysis with monte carlo cross validation showed 13 genes as significant predictors to the response to CGC-11047 treatment (Table 8), which are generally involved in cell motility, cell differentiation, response to stress, and cellular metabolic processes. Higher levels of expression of WASL, CST3, DEAF1 and ACSL3 predicted resistance to CGC-11047 treatment, and higher levels of expression of GCLM, LAMA3, SSRP1, ACYP1, CYLD, PRPF18, AMFR, PPP1R2, and LOH11CR2A predicted increased sensitivity to CGC-11047.

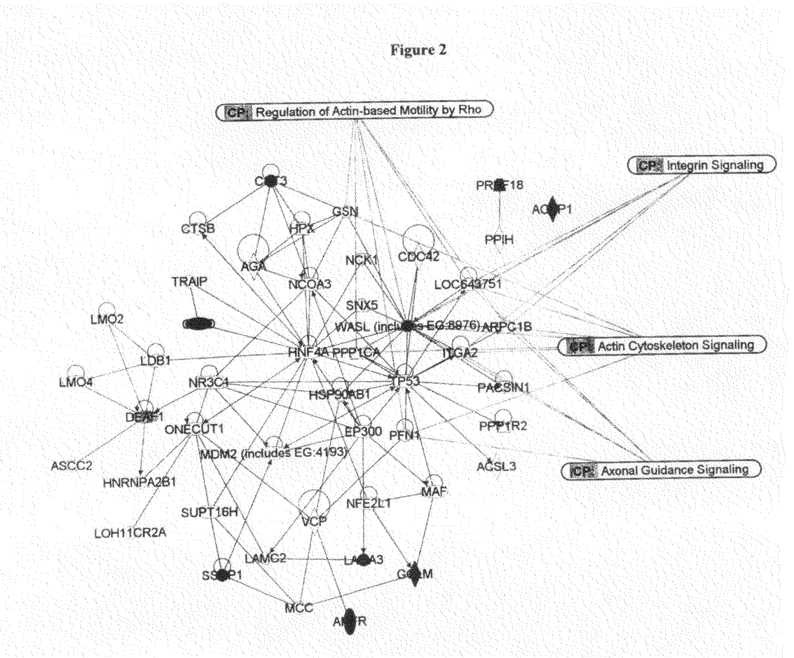

TABLE 8

| | | Predicts | | | GenBank |
|---|---|---|---|---|---|
| | | Sensitivity(S) | Adaptive | | Accession |
| | Affymetrix | or | Spline p- | | No. |
| Gene ID | Probe ID | Resistance(R) | value | q-value | (mRNA) |
| WASL | 205809_s_at | R | 2.53E−05 | 5.55E−03 | BE504979 |
| GCLM | 203925_at | S | 4.72E−05 | 6.37E−03 | NM_002061 |
| CST3 | 201360_at | R | 6.25E−05 | 7.12E−03 | NM_000099 |
| LAMA3 | 203726_s_at | S | 6.65E−05 | 7.12E−03 | NM_000227 |
| SSRP1 | 200956_s_at | S | 1.22E−04 | 8.42E−03 | BE795648 |
| ACYP1 | 205260_s_at | S | 1.24E−04 | 8.42E−03 | NM_001107 |
| CYLD | 221903_s_at | S | 2.89E−04 | 1.14E−02 | BE046443 |
| PRPF18 | 221547_at | S | 2.96E−04 | 1.14E−02 | BC000794 |
| AMFR | 202203_s_at | S | 3.13E−04 | 1.14E−02 | NM_001144 |
| DEAF1 | 209407_s_at | R | 7.88E−04 | 1.37E−02 | AF068892 |
| PPP1R2 | 202166_s_at | S | 1.55E−03 | 1.71E−02 | NM_006241 |
| LOH11CR2A | 210102_at | S | 1.67E−03 | 1.76E−02 | BC001234 |
| ACSL3 | 201661_s_at | R | 1.75E−03 | 1.81E−02 | NM_004457 |

Whereas most of the predictor genes are involved in cellular metabolic processes, some of the genes shared specific functions. Specifically, WASL, LAMA3 and AMFR are all involved in cell motility function; GCLM, LAMA3 and DEAF1 are involved in cell differentiation; GCLM and SSRP1 are involved in response to stress; and CYLD and LOH11CR2A are anti-oncogene/tumor suppressor genes. Network association analyses with Ingenuity showed that 11 of the genes are inter-related, either directly or indirectly through other genes, in networks involved with actin or integrin, likely through migration/motility function of the cells (FIG. 2). WASL is one gene that is directly related with integrin signaling, actin cytoskeleton signaling, regulation of actin-based motility and axonal guidance signaling.

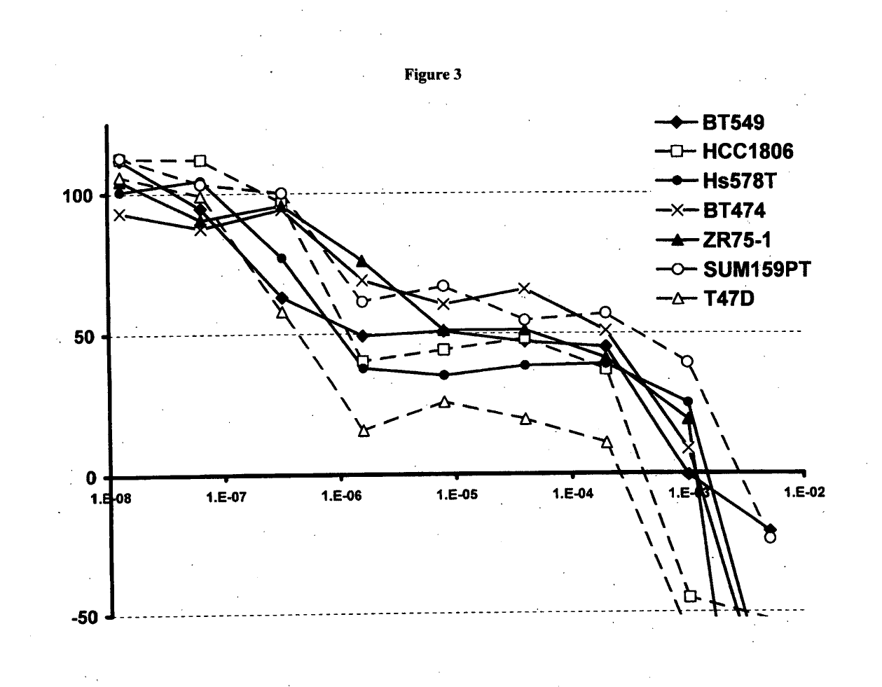

Most of the cell lines when treated with CGC-11047 showed a middle plateau phase between 1 uM and 1 mM range (FIG. 3).

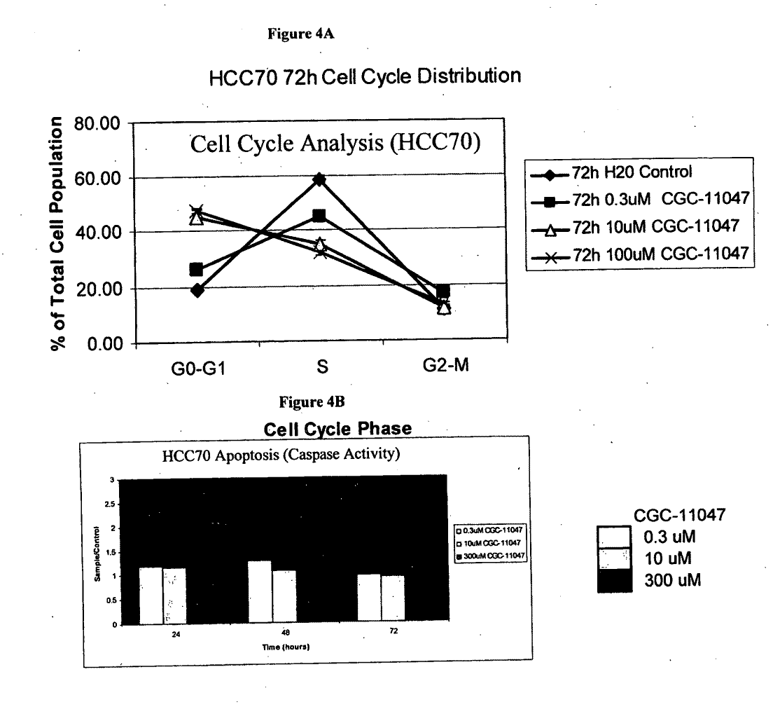

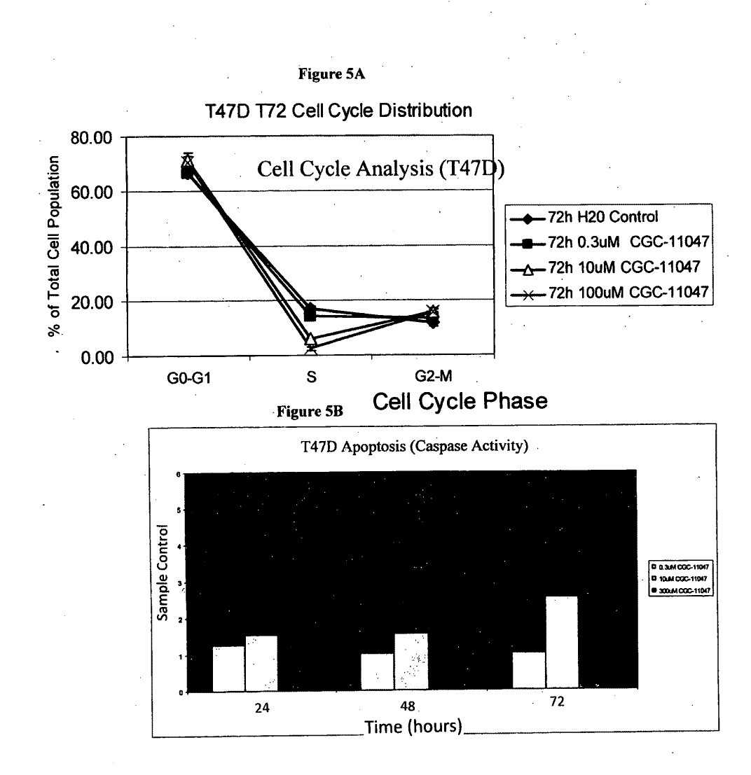

Select sensitive cell lines were treated with CGC-11047 at 0.3, 10 and 300 uM. BrdU incorporation was measured and cell cycle distribution was analyzed at 48 and 72 hrs. Apoptosis was measured using the Promega Caspase Glo 3/7 assay. These cell lines, except T47D, showed that growth inhibition is mainly caused by the delay in cell cycle, a G1 arrest, with very little apoptosis detected (FIG. 4). The T47D cell line, however, showed a significant accumulation of apoptosis, but very little changes in the cell cycle (FIG. 5).

Cell lines that responded to CGC-11047 treatment with a middle plateau phase generally had a TGI (dose required for 0% growth) much higher than the GI₅₀ value. By comparing the expression profiles from these two groups for differentially expressed genes, we generated a list of 264 genes with p<0.05. Analyzing these 264 genes through DAVID functional annotation generated several enriched functional groups. Of interest, the most significant groups of genes are involved in G1/S transition of mitotic cell cycle. The pathway annotation with DAVID also showed that 3 genes (Rb1, FBW7 and CUL1) in the BIOCARTA cyclin E destruction pathway are the top genes differentially expressed between the two groups.

The CCNE and CDK2 complex is needed to phosphorylate Rb, thus freeing E2F1 to activate genes required for G1/S transition. However an F-Box complex composed of FBW7, CUL1 and CDC34 will recruit and phosphorylate CCNE which will eventually lead to the degradation of CCNE by proteosome. When this happens Rb1 inhibits E2F1 and its activation of the genes required for the G1/S transition.

Discussion: Of the 13 predictor genes for the responsiveness to 11047 treatment, 11 of them can be linked to the actin cytoskeleton or integrin-mediated cell motility function either directly or indirectly through interaction with other genes as indicated in the Ingenuity network search.

Cell migration is a fundamental process in tumor metastasis. The integrin family of the receptors is responsible for migration, in part by adhering to the extracellular matrix, and by activating intracellular cascades that promote actin polymerization involved in lamellipodial extension (reviewed by Vandenberg Calif. 2008). One mechanism of integrin-mediated cell migration involves the ability of integrins to regulate the activity of the Rho family of small G proteins. One example of this is Rac activation by alpha4beta1 integrin. Phosphorylation of alpha4beta1 integrin promotes unbinding of the signaling adapter protein paxillin and activation of Rac; dephosphorylation of alpha4beta1 integrin inhibits this process. Two recent reports illustrated a new mechanism of action of the closely related alpha9beta1 integrin to enhance cell migration (deHart et al., 2008; Vandenberg 2008). The alpha9 subunit of integrins may recruit SSAT to focal adhesion; this may relieve repression of Kir channels, thus initiating an outward K+ flow. They hypothesize that the K+ efflux may cause the influx of Ca++, which is known to stimulate migration in other contexts.

Two of the genes, WASL and AMFR, are of particular interest due to their cellular and molecular function reported so far. WASL, also known as N-WASP, is a key regulator of cell migration and actin polymerization. Proteins of the Wiskott-Aldrich Syndrome protein (WASp) family connect signaling pathways to actin polymerization-driven cell motility by interacting with the Arp2/3 complex. Carlier et al. used peptide inhibitors, mutated Grb2, and isolated SH3 domains to demonstrate that N-WASP binds to the SH3 domains of GRB2 and to suggest that Grb2 may activate Arp2/3 complex-mediated actin polymerization downstream from the receptor tyrosine kinase signaling pathway. Recently, Buorguignan et al. reported that N-WASP plays a pivotal role in regulating HA (hyaluronon)-mediated CD44-ErbB2 interaction, beta-catenin signaling, and actin cytoskeleton functions that are required for tumor-specific behaviors and ovarian cancer progression. IHC and quantitative RT-PCR revealed that breast cancer tissues had significantly lower levels of N-WASP compared with normal background mammary tissues. When MDAMB231 cells were stably transfected with N-WASP in vitro, they exhibited a significantly reduced in vitro invasiveness and motility compared with control cells and had increased adhesiveness. (Clin Exp Metastasis 2008; 25:97-108). In our study, WASL is a predictor for resistance with CGC-11047 treatments, i.e., cell lines with higher expression level of WASL tend to be resistant to CGC-11047 treatments, whereas cell lines with lower WASP expression are more sensitive.

AMFR is a tumor motility-stimulating protein secreted by tumor cells. AMFR is one of 189 genes that present a high frequency of intragenic mutations in breast cancer (Sjoblom et al., 2006). The protein encoded by this gene is a glycosylated transmembrane protein and a receptor for autocrine motility factor. Stimulation of AMFR by AMF alters cellular adhesion, proliferation, motility and apoptosis. AMFR is a positive predictor for CGC-11047 sensitivity, therefore patients with high AMFR expression, which generally associate with poor prognosis (Jiang et al., 2006; Hirono et al., 1996), will benefit with CGC-11047 treatment. There is a high degree of correlation of AMFR expression and phospho-Akt on the same breast tumor TMA. In vitro studies with breast cancer cell lines suggest that internalization of cell surface AMFR may be associated with PI3K-dependent activation of Akt and reduction of Cav1 levels in breast tumor cells. Thus PI3K-dependent, Cav1-regulated endocytosis of AMF may represent a cancer cell-specific endocytotic pathway (Kojic et al., 2007).

Most of the cell lines when treated with CGC-11047 showed a middle plateau phase in the mid-treatment of CGC-11047 dose range (approximately 1 uM to 200 uM). Because a dose of 10 uM may not be of clinical interest, it is important to understand this plateau phase. We compared those cell lines with a significant plateau (lasting 2-3 log units) versus others and generated a list of genes that may be associated with this phenomenon. Of them, genes involved with cell cycle control, in particular, the G1/S transition appear to have a strong association with the plateau. Three genes (Rb1, FBW7 and CUL1) from the list are involved in the cyclin E destruction pathway, affecting the transition of G1 to S phase.

This is consistent with our cellular assay where we showed that with the sensitive lines, increasing concentrations of CGC-11047 (at 0.3, 10 and 300 uM) decreases BrdU incorporation rate and induces an apparent G1 arrest (increases G1 fraction and decreases S phase fraction) with very little apoptosis. One exception, the T47D cell line, showed the opposite. This indicates that the G1 cell cycle arrest may be one of the major mechanisms of action of conformationally-restricted polyamines in inhibiting growth of cancer cells.

### Examples 5-12

### In vitro Effect of Conformationally-Restricted Polyamines

The following Examples are provided to illustrate the utility of conformationally-restricted polyamines to inhibit neoplastic cell growth. As noted above, the Examples do not limit the scope of the invention described and claimed herein in any fashion.

Cell lines and media: Human breast cancer cell line MCF7 was grown in Richter's Improved Modified Eagle's Medium supplemented with 10% fetal bovine serum (FBS) and 2.2 g/L sodium bicarbonate. Human brain cancer cell line U251MG-NCI was grown in Dulbecco's Modified Eagle's Medium supplemented with 10% FBS. Human lung cancer cell line A549 was grown in Ham's F-12K medium (Fisher Scientific, Itasca, Ill.), supplemented with 10% FBS and 2 mM L-glutamine. Human colon cancer cell line HT29 was cultured in McCoy's 5A medium (Gibco, BRL, Gaithersburg, Md.), supplemented with 10% FBS. Human prostate cancer cell line PC3 was grown in Dulbecco's Modified Eagles Medium supplemented with 5% FBS. The A549 and MCF7 cell lines were cultured in 100 units/mL penicillin and 100 μg/mL streptomycin. HT29 and U251MG cell lines were grown in 50 μg/mL gentamycin. PC3 cell lines were maintained in 1% antibiotic-antimycotic solution (Sigma, St. Louis, Mo.). The cell cultures were maintained at 37° C. in 5% CO₂/95% humidified air. All cell cultures are available from the American Type Culture Collection, Rockville, Md.

A standardized protocol was used to evaluate these test cultures:

Day 1: Ten standard culture flasks for each drug to be tested were plated with 5×10⁵ cells of a given type in 5 mL of media and allowed to incubate for 16-24 hours at 37° C.

Day 2: Fresh stocks of the compounds to be evaluated are prepared. For each drug, two of the ten culture flasks prepared on Day 1 are used as controls. The control flasks are treated with solvent only. Four flasks for each compound are then treated with serially-diluted concentrations of the compound. The remaining flasks are left untouched. The cells are incubated for 4 hours at 37° C.

After 4 hours the control flasks are counted (2 counts for each flask) and the cells per mL calculated based on the average of the control counts. The cells are then re-plated into six 60 mm dishes for each flask from dilutions based on the cells/mL of the control. (In the various test runs, cell concentrations ranged from approximately 50 to approximately 800 cells per mL.)

Day 15-20: The cells are monitored for colony formation. When visible, the cells are stained with 0.5% crystal violet (in 95% EtOH) and counted. The plating efficiency for each dish is then calculated. The plating efficiencies of the six dishes for each flask are averaged and the standard deviation is calculated. The fraction of cell survival at each concentration is determined based on the controls.

### Example 5

### In vitro Effect of SL-11048 (Compound 57) on MCF7

Following the standard protocol described above, the effect of SL-11048 (Compound 57) on MCF7 cell lines was evaluated. The results revealed that ED₅₀=1.49 μM.

### Example 6

### In vitro Effect of SL-11038 (Compound 23) on MCF7

Following the standard protocol described above, the effect of SL-11038 (Compound 23) on MCF7 cell lines was evaluated. The results showed that ED₅₀=1.34 μM.

### Example 7

### In vitro Effect of SL-11037 (Compound 28) on MCF7

Following the standard protocol described above, the effect of SL-11037 (Compound 28) on MCF7 cell lines was evaluated. The results showed that ED₅₀=1.64 μM.

### Example 8

### In vitro Effect of SL-11043 (Compound 48) on MCF7

Following the standard protocol described above, the effect of SL-11043 (Compound 48) on MCF7 cell lines was evaluated. The results revealed that ED₅₀=1.64 μM.

### Example 9

### In vitro Effect of SL-11047 (Compound 58) on MCF7

Following the standard protocol described above, the effect of SL-11047 (Compound 58) on MCF7 cell lines was evaluated. The results showed that ED₅₀=1.49 μM.

### Example 10

### In vitro Effect of SL-11044 (Compound 47) on MCF7

Following the standard protocol described above, the effect of SL-11044 (Compound 47) on MCF7 cell lines was evaluated. The results showed that ED₅₀=1.79 μM.

### Example 11

### In vitro Effect of 10 μM Concentrations of SL-11033 (13), SL-11027 (12), SL-11034 (36), and SL-11028 (35) on U251MG-NCI Cells

Here, the above-identified compounds were administered in a 10 μM dose to cultures of the human brain cancer cell line U251MG-NCI and evaluated according to the standard protocol described above. At the 10 μM dosage used, SL-11027 (12) displayed marked inhibition of cell growth.

### Example 12

### In vitro Effect of 40 μM Concentrations of SL-11033 (13), SL-11027 (12), SL-11034 (36), and SL-11028 (35) on U251MG-NCI Cells

This Example is identical to Example 7, with the exception that a 40 μM dose was administered. Here, at the 40 μM dosage used, SL-11034 (36) displayed marked inhibition of cell growth.

### Examples 13-24

### ID₅₀ Determination of Conformationally-Restricted Polyamines in Various Cell Lines

Here, a conventional MTT assay was used to evaluate percent cell survival. Exponentially growing monolayer cells were plated in 96-well plates at a density of 500 cells per well and allowed to grow for 24 hours. Serial dilutions of the drugs were added to the wells. Six days after drug treatment, 25 μl of MTT solution (5 mg/ml) was added to each well and incubated for 4 hours at 37° C. Then 100 μl of lysis buffer (20% sodium dodecyl sulfate, 50% DMF, and 0.8% acetic acid, pH 4.7) was added to each well and incubated for an additional 22 hours. A microplate reader (“EMAX”-brand, Molecular Devices, Sunnyvale, Calif.) set at 570 nm was used to determine the optical density of the cultures. Results are expressed as a ratio of the optical density in drug-treated wells to the optical density in wells treated with vehicle only.

The ID₅₀ doses for the compounds tested against the various cell lines are presented in Table 9. The ID₅₀ is the drug concentration that killed 50% of the cultured cells.

TABLE 9
Cytotoxic Activity on Human Tumor Cell Lines

| | ID₅₀(μM) | | | | |
|---|---|---|---|---|---|
| | A549 | HT-29 | PC-3 | MCF7 | U251MG NCl |
| SL-11037 | 0.12 | 1.6 | 7.4 | >31.25 | 0.1 |
| (Cmpd 28) | | | | | |
| SL-11038 | 0.25 | 1.4 | 12.4 | >31.25 | 0.1 |
| (Cmpd 23) | | | | | |
| SL-11043 | 0.1 | 1.5 | >31.25 | 25.5 | 0.1 |
| (Cmpd 48) | | | | | |
| SL-11044 | 0.3 | 1.6 | >31.25 | >31.25 | 0.12 |
| (Cmpd 47) | | | | | |
| SL-11047 | 0.25 | 1.6 | 3.6 | 9.5 | 0.55 |
| (Cmpd 58) | | | | | |
| SL-11048 | 0.26 | 1.4 | 1.4 | >31.25 | 2 |
| (Cmpd 57) | | | | | |

### Example 13

Using the standard MTT protocol described above, cultured HT29 cells were exposed to serial dilutions of compounds 28 (SL11037) and 23 (SL11038). The percent cell survival as compared to cultures exposed to vehicle alone was determined for each concentration of drug. The ID₅₀ for these compounds against HT29 is given in Table 9.

### Example 14

Using the standard MTT protocol described above, cultured HT29 cells were exposed to serial dilutions of compounds 48 (SL11043) and 47 (SL11044). The percent cell survival as compared to cultures exposed to vehicle alone was determined for each concentration of drug. The ID₅₀ for these compounds against HT29 is given in Table 9.

### Example 15

Using the standard MTT protocol described above, cultured HT29 cells were exposed to serial dilutions of compounds 58 (SL11047) and 57 (SL11048). The percent cell survival as compared to cultures exposed to vehicle alone was determined for each concentration of drug. The ID₅₀ for these compounds against HT29 is given in Table 9.

### Example 16

Using the standard MTT protocol described above, cultured U251 MG cells were exposed to serial dilutions of compounds 28 (SL11037) and 23 (SL11038). The percent cell survival as compared to cultures exposed to vehicle alone was determined for each concentration of drug. The ID₅₀ for these compounds against U251 MG is given in Table 9.

### Example 17

Using the standard MTT protocol described above, cultured U251 MG cells were exposed to serial dilutions of compounds 48 (SL11043) and 47 (SL11044). The percent cell survival as compared to cultures exposed to vehicle alone was determined for each concentration of drug. The ID₅₀ for these compounds against U251 MG is given in Table 9.

### Example 18

Using the standard MTT protocol described above, cultured U251 MG cells were exposed to serial dilutions of compounds 58 (SL11047) and 57 (SL11048). The percent cell survival as compared to cultures exposed to vehicle alone was determined for each concentration of drug. The ID₅₀ for these compounds against U251 MG is given in Table 9.

### Example 19

Using the standard MTT protocol described above, cultured A549 cells were exposed to serial dilutions of compounds 28 (SL11037) and 23 (SL11038). The percent cell survival as compared to cultures exposed to vehicle alone was determined for each concentration of drug. The ID₅₀ for these compounds against A549 is given in Table 9.

### Example 20

Using the standard MTT protocol described above, cultured A549 cells were exposed to serial dilutions of compounds 48 (SL11043) and 47 (SL11044). The percent cell survival as compared to cultures exposed to vehicle alone was determined for each concentration of drug. The ID₅₀ for these compounds against A549 is given in Table 9.

### Example 21

Using the standard MTT protocol described above, cultured A549 cells were exposed to serial dilutions of compounds 58 (SL11047) and 57 (SL11048). The percent cell survival as compared to cultures exposed to vehicle alone was determined for each concentration of drug. The ID₅₀ for these compounds against A549 is given in Table 9.

### Example 22

Using the standard MTT protocol described above, cultured PC3 cells were exposed to serial dilutions of compounds 28 (SL11037) and 23 (SL11038). The percent cell survival as compared to cultures exposed to vehicle alone was determined for each concentration of drug. The ID₅₀ for these compounds against PC3 is given in Table 9.

### Example 23

Using the standard MTT protocol described above, cultured PC3 cells were exposed to serial dilutions of compounds 48 (SL11043) and 47 (SL11044). The percent cell survival as compared to cultures exposed to vehicle alone was determined for each concentration of drug. The ID₅₀ for these compounds against PC3 is given in Table 9.

### Example 24

Using the standard MTT protocol described above, cultured PC3 cells were exposed to serial dilutions of compounds 58 (SL11047) and 57 (SL11048). The percent cell survival as compared to cultures exposed to vehicle alone was determined for each concentration of drug. The ID₅₀ for these compounds against PC3 is given in Table 9.

1-13. (canceled)

14. A method for identifying a basal-type cancer patient, comprising: (a) measuring expression level of one gene selected from the group consisting of genes encoding SCNN1A, CA12, PRKX, TFF3, HNF3A, MYB, GABRP, ESR1, AGR2, GATA3, FOXC1, and EN1, in a sample from the patient; and (b) comparing the expression level of the gene from the patient with expression level of the gene in a normal tissue sample or a reference expression level, wherein an increase of expression of one gene selected from the group consisting of the genes encoding PRKX, GABRP, FOXC1, and EN1 and a decrease of expression of one gene selected from the group consisting of the genes encoding SCNN1A, CA12, TFF3, HNF3A, MYB, ESR1, AGR2, and GATA3 indicates the patient has basal-type cancer.

15. The method of claim 14, wherein a decrease of expression of one gene selected from the group consisting of the genes encoding PRKX, GABRP, FOXC1, and EN1 and an increase of expression of one gene selected from the group consisting of the genes encoding SCNN1A, CA12, TFF3, HNF3A, MYB, ESR1, AGR2, and GATA3 indicates the patient does not have basal-type cancer.

16. The method of claim 15, further comprising (c) measuring expression level of at least two genes selected from the group consisting of SCNN1A, CA12, PRKX, TFF3, HNF3A, MYB, GABRP, ESR1, AGR2, GATA3, FOXC1, and EN1, in a sample from the patient, and (d) comparing the expression level of step (c) with expression levels of the genes in a normal tissue sample or a reference expression level, or an average expression level in a panel of normal cell lines or cancer cell lines.

17. The method of claim 16, further comprising (d) measuring the expression level of each gene in the group consisting of SCNN1A, CA12, PRKX, TFF3, HNF3A, MYB, GABRP, ESR1, AGR2, GATA3, FOXC1, and EN1 in a sample from the patient; and (e) comparing the expression level of step (d) with expression levels of the genes in a normal tissue sample or a reference expression level, or a average expression level in a panel of normal cell lines or cancer cell lines.

18. A method for identifying a basal or luminal phenotype of a cell, comprising: (a) measuring expression level of one gene selected from the group consisting of genes encoding SCNN1A, CA12, PRKX, TFF3, HNF3A, MYB, GABRP, ESR1, AGR2, GATA3, FOXC1, and EN1, in a patient sample; and (b) comparing the expression level of gene from a sample with expression level of the gene in a normal tissue sample or a reference expression level, wherein an increase of expression in the patient sample of one gene selected from the group consisting of the genes encoding PRKX, GABRP, FOXC1, and EN1, indicates the cell has a basal phenotype and an increase of expression of one gene selected from the group consisting of the genes encoding SCNN1A, CA12, TFF3, HNF3A, MYB, ESR1, AGR2, and GATA3, indicates the cell has a luminal phenotype.

19. The method of claim 18, wherein a decrease of expression in the patient sample of one gene selected from the group consisting of the genes encoding PRKX, GABRP, FOXC1, and EN1, indicates the cell has a luminal phenotype, and a decrease of expression of one gene selected from the group consisting of the genes encoding SCNN1A, CA12, TFF3, HNF3A, MYB, ESR1, AGR2, and GATA3, indicates the cell has a basal phenotype.

20-32. (canceled)

33. An assay to detect modulated expression of a gene as a predictor or marker of basal-type cancers, the assay comprising:
RT-PCR primers dimensioned and configured to detect transcription level of one or more genes selected from the group consisting of SCNN1A, CA12, PRKX, TFF3, HNF3A, MYB, GABRP, ESR1, AGR2, GATA3, FOXC1, and EN1.

34. An assay to detect modulated expression of a gene as a predictor or marker of basal-type cancers, the assay comprising immunochemical reagents to detect a polypeptide expressed by one or more genes selected from the group consisting of SCNN1A, CA12, PRKX, TFF3, HNF3A, MYB, GABRP, ESR1, AGR2, GATA3, FOXC1, and EN1.

35. A compound to treat a cell that exhibits modulated expression of a gene that is a predictor or maker of basal-type cancer, wherein the compound is configured to inhibit expression of one or more genes selected from the group consisting of SCNN1A, CA12, PRKX, TFF3, HNF3A, MYB, GABRP, ESR1, AGR2, GATA3, FOXC1, and EN1.

36. The compound of claim 35 to treat a cell that exhibits modulated expression of a gene that is a predictor or maker of basal-type cancer, wherein the compound comprises an siRNA or an antisense oligonucleotide.

37. The compound of claim 36 wherein the inhibitor is encoded on a viral vector that is dimensioned and configured to produce the antisense oligonucleotide or siRNA.

38. The compound of claim 35 to treat a cell that exhibits modulated expression of a gene that is a predictor or maker of basal-type cancer, wherein the compound comprising an aptamer.

39. The compound of claim 35 to treat a cell that exhibits modulated expression of a gene that is a predictor or maker of basal-type cancer, wherein the compound comprising an antibody.

40-41. (canceled)
